(19) United States
(12) Patent Application Publication
(10) Pub. No.: US 2025/0114095 A1
(43) Pub. Date: Apr. 10, 2025

(54) SYSTEMS, DEVICES, AND METHODS FOR DELIVERY OF OTOMY SUPPORT AND ANASTOMOSIS CREATION DEVICES

(71) Applicant: G.I. Windows, Inc., Westwood, MA (US)

(72) Inventors: Dana Zitnick, Westwood, MA (US); Michael Boutillette, Sudbury, MA (US); Jonathan P. Boduch, Quincy, MA (US); Dane T. Seddon, Boston, MA (US)

(21) Appl. No.: 18/922,011

(22) Filed: Oct. 21, 2024

Related U.S. Application Data
(63) Continuation of application No. PCT/US2024/047958, filed on Sep. 23, 2024.
(60) Provisional application No. 63/539,926, filed on Sep. 22, 2023.

(51) Int. Cl. A61B 17/11

(52) U.S. Cl. CPC A61B 17/11

(57) ABSTRACT

A delivery system includes a reloadable handpiece to which cartridges of various configurations can be attached and operated to deliver otomy control devices, magnetic compression anastomosis devices, and/or other devices for anastomosis procedures. The handpiece generally includes a deployment mechanism (e.g., including a pusher) configured to control deployment of the at least one implant from a distal end of an attached cartridge and at least one actuator allowing a user to operate the deployment mechanism. The at least one actuator may include an implant delivery actuator and a suture release actuator.

### CROSS-REFERENCE TO RELATED APPLICATION(S)

This patent application is a continuation of International Patent Application No. PCT/US2024/047958 entitled SYSTEMS, DEVICES, AND METHODS FOR DELIVERY OF OTOMY SUPPORT AND ANASTOMOSIS CREATION DEVICES filed Sep. 23, 2024 (Attorney Docket No. 121326-12603), which claims the benefit of U.S. Patent Application No. 63/539,926 entitled APPARATUS FOR DELIVERY OF OTOMY SUPPORT AND ANASTOMOSIS DEVICES filed Sep. 22, 2023, each of which is hereby incorporated herein by reference in its entirety.

The subject matter of this patent application may be related to the subject matter of commonly-owned U.S. patent application Ser. No. 18/229,988 entitled MAGNETIC COMPRESSION ANASTOMOSIS DEVICES WITH MULTIPIECE INTERNAL VERTEBRAE SUPPORT STRUCTURES filed Aug. 3, 2023 (U.S. Patent Application Publication No. US 2024/0065694) and commonly-owned U.S. patent application Ser. No. 18/230,066 entitled MAGNETIC COMPRESSION ANASTOMOSIS DEVICE WITH MULTIPIECE VERTEBRA filed Aug. 3, 2023 (U.S. Patent Application Publication No. US 2024/0041460), each of which is hereby incorporated by reference in its entirety.

The subject matter of this patent application also may be related to the subject matter of commonly-owned U.S. patent application Ser. No. 18/384,022 entitled SYSTEMS AND METHODS FOR PRESERVING AND MANIPULATING OF ACUTE OTOMIES filed Oct. 26, 2023 (U.S. Patent Application Publication No. US 2024/0138839), which claims the benefit of commonly-owned U.S. Provisional Patent Application No. 63/419,509 entitled SYSTEMS AND METHODS FOR PRESERVING AND MANIPULATING OF ACUTE OTOMIES filed Oct. 26, 2022 and commonly-owned U.S. Provisional Patent Application No. 63/435,724 entitled SYSTEMS AND METHODS FOR PRESERVING AND MANIPULATING OF ACUTE OTOMIES filed Dec. 28, 2022, each of which is hereby incorporated herein by reference in its entirety.

### FIELD OF INVENTION

The invention relates to a universal delivery device usable with cartridges of various configurations to deliver otomy control devices, magnetic compression anastomosis devices, and/or other devices for anastomosis procedures.

### BACKGROUND

Bypasses of the gastroenterological (GI), cardiovascular, or urological systems traditionally were often formed by cutting holes (i.e., acute otomies) in tissues at two locations and joining the holes with sutures or staples to create an anastomosis. A bypass (also called an anastomosis) is typically placed to route fluids (e.g., blood, nutrients) between healthier portions of the system, while bypassing diseases or malfunctioning or even sometimes healthy tissues as necessary for the surgical procedure being performed. The procedure is typically invasive, and subjects a patient to risks such as bleeding, infection, pain, and adverse reaction to anesthesia. Additionally, a bypass created with sutures or staples can be complicated by post-operative leaks and adhesions and leaves a foreign body behind in the patient. Leaks may result in infection or sepsis, while adhesions can result in complications such as bowel strangulation and obstruction. Leaving foreign bodies behind can result in chronic inflammation, infection, obstruction, etc. While traditional bypass procedures can be completed with an endoscope, laparoscope, or robot, it can be time consuming to join the holes cut into the tissues. Furthermore, such procedures require specialized expertise and equipment that is not available at many surgical facilities.

As an alternative to sutures or staples, surgeons can use mechanical couplings or magnets to create a compressive anastomosis between tissues. For example, compressive couplings or paired magnets can be delivered to tissues to be joined. Because of the strong compression, the tissue trapped between the couplings or magnets is cut off from its blood supply. Under these conditions, the tissue becomes necrotic and degenerates, and at the same time, new tissue grows around points of compression, e.g., on the edges of the coupling. With time, the coupling can be removed, leaving a healed anastomosis between the tissues.

Nonetheless, the difficulty of placing the magnets or couplings limits the locations that compressive anastomosis can be used. In most cases, the magnets or couplings have to be delivered as two separate assemblies, requiring either an open surgical field or a bulky delivery device. For example, existing magnetic compression devices are limited to structures small enough to be deployed with a delivery conduit e.g., an endoscopic instrument channel or laparoscopic port. When these smaller structures are used, the formed anastomosis is small and suffers from short-term patency. Furthermore, placement of the magnets or couplings can be imprecise, which can lead to anastomosis formation in locations that is undesirable or inaccurate.

Tissues of different thicknesses require different pressures to puncture the tissues and/or bring magnetic anastomosis devices close enough together to mate. Sharp pressure profiles may cause abrupt transitions between healthy and necrotic tissues, which can cause poor sealing of the anastomosis.

Thus, there still remains a clinical need for reliable devices and delivery systems in minimally invasive procedures that facilitate compression anastomosis formation between tissues in the human body in as few steps as possible.

### SUMMARY

Embodiments include a universal delivery device, cartridges for use with a universal delivery device, and methods of treatment using a universal delivery device and one or more cartridges.

In accordance with one embodiment, a system for delivery of one or more implants used in the protection of otomies and the creation of magnetic compression anastomoses in a target anatomy comprises a reloadable handpiece and one or more cartridges containing the one or more implants.

In various alternative embodiments, each cartridge may contain at least one implant selected from the group consisting an otomy control device and a magnetic compression anastomosis device and the reloadable handpiece may have an interface (e.g., a shaft) through which each of the one or more cartridges can be attached to and operated by the handpiece, the handpiece including a deployment mechanism configured to control deployment of the at least one implant from a distal end of an attached cartridge and at least one actuator allowing a user to operate the deployment mechanism. The at least one actuator may include an implant delivery actuator and a suture release actuator. The deployment mechanism may include a pusher configured to push the at least one implant from the distal end of the deployment channel. The deployment mechanism may include a ratcheting feature to control advancement of the at least one implant. The deployment mechanism may include a linkage drive mechanism for controlling actuation and release operations. The deployment mechanism may include a cable drive mechanism for controlling actuation and release operations. The deployment mechanism may include one or more motors for controlling actuation and release operations.

Different cartridge configurations may include only one implant (e.g., an otomy control device implant or a magnetic compression anastomosis implant), two implants (e.g., a distal implant and a proximal implant), more than two implants (e.g., multiple otomy control implants and/or magnetic compression anastomosis implants). Cartridges may be configured with otomy alignment features, without otomy alignment features, and/or thru deployment features. The magnetic compression anastomosis device may be a self-assembling magnetic compression anastomosis device having a substantially linear configuration within the cartridge and an annular deployed configuration (e.g., a Flexagon or other self-assembling device). The otomy control device may be an OTOLoc™ or other otomy control device.

In accordance with another embodiment, an anastomosis procedure cartridge contains at least one implant selected from the group consisting an otomy control device and a magnetic compression anastomosis device, wherein the cartridge is configured to attach to an interface of a reloadable handpiece by which the cartridge can be operated to deliver and release the at least one implant from a distal end of the cartridge. Such a cartridge may include only one implant (e.g., an otomy control device implant or a magnetic compression anastomosis implant), two implants (e.g., a distal implant and a proximal implant), more than two implants (e.g., multiple otomy control implants and/or magnetic compression anastomosis implants). Cartridges may be configured with otomy alignment features, without otomy alignment features, and/or thru deployment features. The magnetic compression anastomosis device may be a self-assembling magnetic compression anastomosis device having a substantially linear configuration within the cartridge and an annular deployed configuration (e.g., a Flexagon or other self-assembling device). The otomy control device may be an OTOLoc™ or other otomy control device.

In accordance with another embodiment, a reloadable handpiece comprises an interface configured to attach to and operate any of the herein described anastomosis procedure cartridges, the handpiece including a deployment mechanism configured to control deployment of the at least one implant from a distal end of an attached cartridge, the deployment mechanism including at least one actuator (e.g., trigger) for a user to operate the deployment mechanism. The at least one actuator may include an implant delivery actuator and a suture release actuator. The deployment mechanism may include a pusher configured to push the at least one implant from the distal end of the deployment channel. The deployment mechanism may include a ratcheting feature to control advancement of the at least one implant. The deployment mechanism may include a linkage drive mechanism for controlling actuation and release operations. The deployment mechanism may include a cable drive mechanism for controlling actuation and release operations. The deployment mechanism may include one or more motors for controlling actuation and release operations.

In accordance with another embodiment, a kit comprises a plurality of the herein described anastomosis procedure cartridges having at least two or more different implant configurations.

In accordance with another embodiment, a method of delivering at least one anastomosis procedure implant comprises attaching an anastomosis procedure cartridge to a reloadable handpiece, the cartridge containing at least one implant selected from the group consisting an otomy control device and a magnetic compression anastomosis device, the handpiece having an interface through which the cartridge attaches to and is operated by the handpiece, the handpiece including a deployment mechanism configured to control deployment of the at least one implant from a distal end of an attached cartridge; and operating at least one actuator (e.g., a trigger) of the handpiece to deploy the at least one implant from the distal end of the attached cartridge. The cartridge may include a plurality of implants, and operating the at least one actuator (e.g., a trigger) may involve at least a first actuation to perform a first deployment operation that deploys a first implant and a second actuation to perform a second deployment operation that deploys a second implant.

Additional embodiments may be disclosed and claimed.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

Those skilled in the art should more fully appreciate advantages of various embodiments of the invention from the following “Description of Illustrative Embodiments,” discussed with reference to the drawings summarized immediately below.

FIG. 1 shows a magnet assembly delivered through an endoscope instrument channel such that the individual magnets self-assemble into a larger magnetic structure—in this particular case, an octagon.

FIG. 2A shows magnet assemblies that have been delivered and deployed to adjacent tissues.

FIG. 2B shows the two magnet assemblies coupled together by magnetic attraction, capturing the intervening tissue. In some instances, the endoscope can be used to cut through the circumscribed tissue.

FIG. 3 shows several potential anatomical targets for anastomosis formation: Arrow A is stomach to small intestine, Arrow B is small intestine to large intestine, Arrow C is small intestine to small intestine, Arrow D is large intestine to large intestine, and Arrow E is stomach to large intestine.

FIG. 4A shows one embodiment of delivery using two endoscopes (colonoscope and enteroscope or gastroscope) to deliver magnet assemblies.

FIG. 4B shows another embodiment of delivery using two upper endoscopes both sharing per-oral entry to deliver magnet assemblies.

FIG. 5 shows another embodiment of delivery using a single endoscope to sequentially deliver magnet assemblies.

FIG. 6 shows another embodiment of delivery using endoscopic ultrasound guided needle delivery of one magnet assembly into lumen #1 followed by deployment to of the second magnet assembly in lumen #2.

FIG. 7 shows the creation of a preliminary anastomosis to serve as a conduit for deeper endoscope delivery in order to create subsequent multiple anastomoses.

FIG. 8 shows laparoscopic magnet device delivery into a lumen (stomach, in this example).

FIG. 9A shows endoscopic ultrasound guided needle delivery of a magnet assembly into the gallbladder which then couples with a second magnet assembly in the stomach or duodenum as shown in FIG. 9B.

FIG. 10 shows stent deployment between the gallbladder and either the stomach or duodenum.

FIG. 11 shows another embodiment of an intra-gallbladder magnet assembly that is a balloon that fills with fluid, gas, or magnetic material. This balloon is tethered to the endoscope and is initially delivered through an endoscopic ultrasound guided needle.

FIG. 12 shows endoscopic ultrasound guided needle delivery of a magnet assembly into the bile duct.

FIG. 13 shows magnet assembly delivery into the bile duct through endoscopic retrograde cholangiopancreatography techniques.

FIG. 14 shows coupling of the intra-bile duct magnet assembly with a second magnet assembly deployed either in the stomach (A) or duodenum (B).

FIG. 15 shows another embodiment of bile duct magnetic anastomosis in which a hinged magnetic bile duct stent swings back onto itself by magnetic attraction to form an anastomosis between the bile duct and duodenum.

FIG. 16 shows a magnetic stent that can be delivered into the pancreatic duct. The stent can be coupled with a magnet in the stomach (A) or in the duodenum (B) to create a drainage anastomosis for the pancreatic duct.

FIG. 17 shows a magnetic assembly that is delivered into a peripancreatic collection (dotted structure) using endoscopic ultrasound guided needle/catheter delivery which then couples with a second magnet assembly deployed in the stomach.

FIG. 18 shows different targets for anastomoses between the urinary system and the gastrointestinal system: renal calyx (A), ureter (B), and bladder (C).

FIG. 19 shows magnet assemblies in adjacent blood vessels to couple and create a vascular anastomosis.

FIG. 20 shows magnet assemblies in different parts of the respiratory system to create anastomoses between adjacent bronchioles.

FIG. 21 shows an external magnet assembly and an internal magnet assembly within the gastrointestinal tract used to create a surgical stoma for fecal drainage.

FIG. 22 schematically shows an exemplary apparatus for controlling an acute otomy.

FIGS. 23A-23I schematically show the procedural steps for creation, control, and healing of an acute otomy.

FIG. 24 schematically shows a silicone grommet identifying the intraluminal and extraluminal flanges and how the grommet sits transmurally in the tissue.

FIG. 25 schematically shows a nitinol tubing/wire hybrid grommet.

FIG. 26 schematically shows a nitinol tubing/wire hybrid grommet.

FIGS. 27A-27B schematically show a nitinol tubing/wire hybrid grommet.

FIGS. 28A-28B schematically show nitinol wire array clamps.

FIG. 29 schematically shows an inflatable grommet.

FIGS. 30A-30B schematically show perspective views of nitinol tubing apparatuses for securing an otomy.

FIGS. 31A-31B schematically show perspective views of nitinol tubing apparatuses for securing an otomy.

FIGS. 32A-32B schematically show perspective views of nitinol tubing apparatuses for securing an otomy.

FIGS. 33A-33B schematically show perspective views of nitinol tubing apparatuses for securing an otomy.

FIGS. 34A-34B schematically show perspective views of nitinol tubing apparatuses for securing an otomy.

FIGS. 35A-35B schematically show perspective views of nitinol tubing apparatuses for securing an otomy.

FIGS. 36A-36B schematically show perspective views of nitinol tubing apparatuses for securing an otomy.

FIGS. 37A-37B schematically show perspective views of nitinol tubing apparatuses for securing an otomy.

FIGS. 38A-38B schematically show perspective views of nitinol tubing apparatuses for securing an otomy.

FIGS. 39A-39B schematically show perspective views of nitinol tubing apparatuses for securing an otomy.

FIGS. 40A-40B schematically show perspective views of nitinol tubing apparatuses for securing an otomy.

FIGS. 41A-41B schematically show perspective views of nitinol tubing/wire hybrid apparatuses for securing an otomy.

FIGS. 42A-42B schematically show perspective views of nitinol tubing/wire hybrid apparatuses for securing an otomy.

FIG. 43 schematically shows a view of a nitinol tubing/wire hybrid apparatus for securing an otomy.

FIGS. 44A-44B schematically show perspective views of ratcheting securing apparatuses for securing an otomy.

FIGS. 45A-45B schematically show perspective views of a circular securing apparatus for securing an otomy.

FIGS. 46A-46B schematically show a stent-style nitinol array apparatus for securing an otomy.

FIGS. 47A-47B schematically show a stent-style nitinol array apparatus for securing an otomy.

FIGS. 48A-48B schematically show a stent-style nitinol array spiraled wire apparatus for securing an otomy.

FIGS. 49A-49B schematically show an inflatable grommet apparatus for securing an otomy.

FIGS. 50A-50B schematically show a two-piece suture clamp apparatus for securing an otomy.

FIGS. 51A-51B schematically show a snap lock apparatus for securing an otomy.

FIGS. 52A-52B schematically show a sliding arms otomy clip for securing an otomy.

FIG. 53 schematically shows a foam grommet apparatus for securing an otomy.

FIG. 54 schematically shows a clip secured grommet otomy clip apparatus for securing an otomy.

FIG. 55 schematically shows a coil compressed grommet apparatus for securing an otomy.

FIGS. 56A-56B schematically show perspective views of an otomy clip apparatus for securing an otomy.

FIGS. 57A-57D schematically show coiled wire concepts for securing an otomy.

FIG. 58 schematically shows an adhesive reinforced apparatus for securing an otomy.

FIGS. 59A-59B schematically show an otomy clip patch for securing an otomy.

FIGS. 60A-60B schematically show an otomy clip puncture patch for securing an otomy.

FIG. 61 schematically shows a crimped wire form otomy clip for securing an otomy.

FIG. 62 is a schematic diagram showing a universal delivery device in accordance with certain embodiments.

FIG. 63 is a schematic diagram showing some cartridge configurations usable with the handpiece of FIG. 62.

FIG. 64 is a schematic diagram showing operation of a cartridge containing an OTOLoc distal to a Flexagon without otomy alignment in accordance with certain embodiments.

FIG. 65 is a schematic diagram showing one cartridge layout in accordance with certain embodiments.

FIG. 66 is a schematic diagram showing one cartridge containing an OTOLoc distal to a Flexagon in accordance with certain embodiments.

FIG. 67 is a perspective view of the handpiece of FIG. 62.

FIG. 68 is a side-view of the handpiece of FIG. 62.

FIG. 69 is an annotated version of the side-view of FIG. 68 highlighting certain components and dimensions of this particular embodiment.

FIGS. 70-71 are schematic diagrams showing a linkage drive mechanism for controlling actuation and release operations.

FIGS. 72-73 are schematic diagrams showing a cable drive mechanism for controlling actuation and release operations.

FIG. 74 is a schematic diagram showing deployment of an OTOLoc followed by deployment of a Flexagon similar to that of FIG. 64 but with reference to the components of the universal delivery device shown in FIG. 69.

FIG. 75 shows a universal delivery device and a cartridge pre-operation, in accordance with certain embodiments.

FIG. 76 shows the cartridge of FIG. 75 being installed onto the universal delivery device.

FIG. 77 shows the cartridge of FIG. 76 fully installed on the universal delivery device.

FIG. 78 shows presentation of the enterotomy towards the OTOLoc cartridge tip with a grasper.

FIG. 79 shows insertion of the shoehorn tip of the cartridge into the enterotomy while apply light tension from the graspers.

FIG. 80 shows deployment of the distal flange of the OTOLoc.

FIG. 81 shows deployment of the proximal flange of the OTOLoc.

FIG. 82 shows the fully released OTOLoc device.

FIG. 83 shows insertion of the Flexagon shaft into the otomy through the OTOLoc.

FIG. 84 shows partial deployment of the Flexagon.

FIG. 85 shows full deployment of the Flexagon.

FIG. 86 shows tension applied to the suture lines to seat the Flexagon against the distal flange of the OTOLoc.

FIG. 87 shows transfer of control of the suture lines to a grasper and release of the suture lines.

FIG. 88 shows two otomy sites joined via OTOLoc and Flexagon devices.

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

Embodiments generally relate to a universal delivery device usable with cartridges of various configurations to deliver otomy control devices, magnetic compression anastomosis devices, and/or other devices for anastomosis procedures. Without limitation, a magnetic compression anastomosis device can include a single element (e.g., a device such as a bar, circular, or annular device formed of a solid magnetic material or filled with or containing magnetic material) or multiple elements (e.g., multiple pieces or particles that can act as or form a magnetic compression anastomosis device such as through self-assembly or through interaction with another magnetic compression anastomosis device). A magnetic material can be a magnetized material or a non-magnetized material (e.g., ferrous or otherwise able to interact magnetically with a magnetized material). It should be noted that, although many embodiments are described below with reference to self-assembling magnetic compression anastomosis devices, embodiments generally are not limited to self-assembling devices. Thus, for example, two magnetic compression anastomosis devices that interact to form an anastomosis can include two single-element devices, two multiple-element devices, or one single-element device and one multiple-element device; can include two magnetized devices, one magnetized device and one non-magnetized device, or devices that have both magnetized and non-magnetized pieces; and/or can include two self-assembling devices, two non-self-assembling devices, or one self-assembling device and one non-self-assembling device.

Self-assembling magnetic anastomosis addresses several of the historical disadvantages of traditional anastomosis such as allowing a surgical-quality anastomosis in a minimally-invasive fashion using devices that reproducibly re-assemble into a larger magnet structure of a predetermined shape in vivo. Certain self-assembling magnetic compression anastomosis devices are designed to allow the devices to consistently self-assemble into the correct shape upon deployment, which greatly reduces the risks of surgical complications due to misshapen devices or premature detachment and also reduces the risks associated with surgical access and ensure that the anastomosis is formed with the correct geometric attributes. Overall, this ensures the patency of the anastomosis.

Thus, as described herein, embodiments include flexible linear magnetic devices comprising linked magnetic multipole segments that, when extruded from the end of a deployment channel or lumen, self-assemble to form a rigid, multipolar polygonal ring magnet (PRM; generally “magnetic device”). The self-assembly can be directed, for example, by the configuration of magnets, rollers, flex elements, vertebra skins, or other mechanism that is capable of returning to a pre-determined shape. Generally speaking, the physical and magnetic structure of the deployed magnetic devices is such that when two magnetic devices approach one another, there is a rapidly strengthening attractive magnetic interaction, which creates a coupling between the magnetic devices. In some instances, it is necessary to pre-align the complementary devices, however, in other instances the devices self-align by undergoing fast in-plane rotation with respect to one another, as discussed in detail below. As described in detail below, systems including the magnetic devices may include an endoscope having sensors that allow the endoscope to sense the position of a mating magnetic device or another endoscope that will deploy the mating device.

When deployed in adjacent tissues, for example adjacent organs or different regions of the same organ, the coupled magnetic devices create a compressive ring that can be surgically opened, or allowed to form an anastomosis without further intervention. When paired devices are left alone, the compressive force against the tissues collapse the vasculature and extrude fluids in the tissues, further reducing the distance between the devices and increasing the magnetic attraction. With time, the coupled devices eventually couple completely and fall away, leaving a formed anastomosis. This cascade begins when the devices approach within “capture range,” whereby their mutually-attractive forces are sufficient to align the devices, trap the intervening tissue, and resist the natural pliancy of the tissues as well as the motion of the tissue under normal physiologic function.

Overall, the design specifications of the devices depend on the patient and the intended anastomosis. The design specifications may include: required capture range, desired effective inner and outer diameters of the deployed polygonal rings (e.g., as defined by the desired anastomosis size and instrument passage), thickness of the target tissue, and the inner diameter of guiding channel and the smallest radius of curvature to which the guiding channel may be bent and through which the magnets must pass. Once the design specifications are chosen, corresponding magnetic device designs can be determined, such as polygon-side-count and length, and the maximum lateral dimensions of the flexible linear magnetic structure that will be deployed through the delivery instrument.

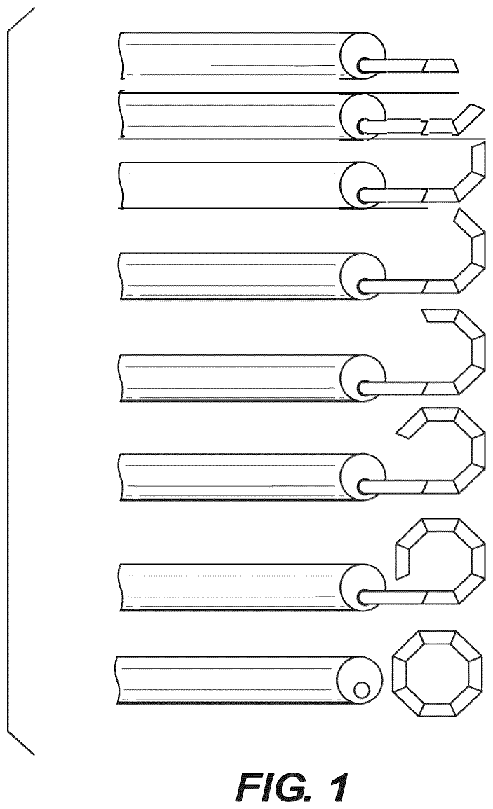

Deployment of a device 100 is generally illustrated in FIG. 1. When used with the techniques described herein, the devices allow for the delivery of a larger magnetic structures than would otherwise be possible via a small delivery conduit, such as in a standard endoscope, if the devices were deployed as a completed assembly. Larger magnet structures, in turn, allow for the creation of larger anastomoses that are more robust, and achieve greater surgical success. Because the magnetic devices are generally radiopaque and echogenic, the devices generally can be positioned using fluoroscopy, direct visualization (trans-illumination or tissue indentation), and ultrasound, e.g., endoscopic ultrasound. The devices can also be ornamented with radiopaque paint or other markers to help identify the polarity of the devices during placement. In some embodiments, the devices can be positioned by use of sensors located in proximity to the delivery lumen and able to sense the position of a mating device, e.g., using a Reed switch or a Hall-effect sensor.

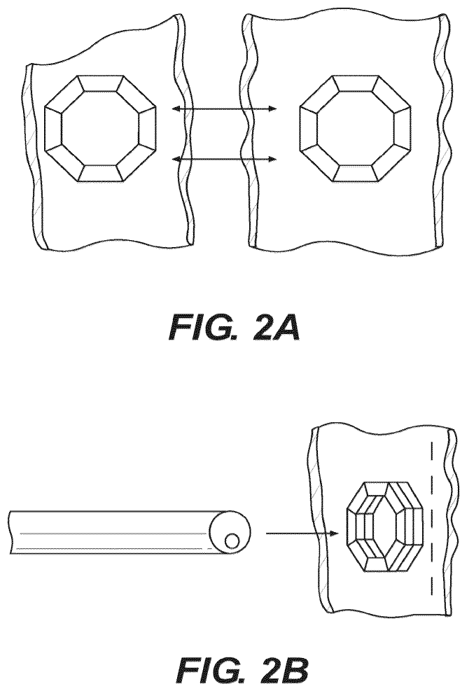

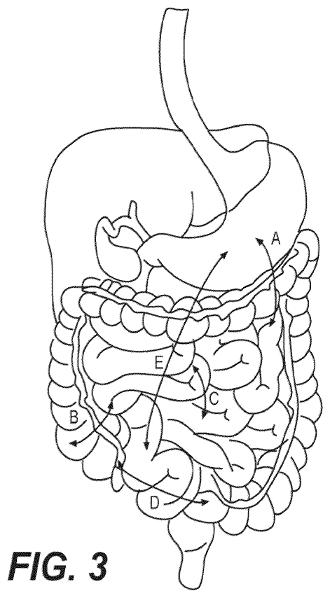

In general, as shown in FIG. 2A, a magnetic anastomosis procedure involves placing a first and a second magnetic structure adjacent to targeted tissues, thus causing the tissues to come together. The magnetic devices are generally deployed so that that opposite poles of the magnets will attract and bring the tissues together. The two devices may both be deployed inside the body, or one may be deployed inside the body and the other outside the body. Once the magnets have been deployed, the tissues circumscribed by the magnetic structures can be cut to provide an immediate anastomosis, as shown in FIG. 2B. In other embodiments, the tissues circumscribed by the devices will be allowed to necrose and degrade, providing an opening between the tissues. While the figures and structures of the disclosure are primarily concerned with annular or polygonal structures, it is to be understood that the delivery and construction techniques described herein can be used to make a variety of deployable magnetic structures. For example, self-assembling magnets can re-assemble into a polygonal structure such as a circle, ellipse, square, hexagon, octagon, decagon, or other geometric structure creating a closed loop. The devices may additionally include handles, suture loops, barbs, and protrusions, as needed to achieve the desired performance and to make delivery (and removal) easier.

As described with respect to the figures, a self-assembling magnetic anastomosis device can be placed with a number of techniques, such as endoscopy, laparoscopy, or with a catheter (e.g., not with direct visualization, fluoro, etc.). Regardless of method of device delivery, it is important to note that the procedure for creating the anastomosis can be terminated without perforation of tissue after confirmation of magnet coupling. As described previously, the compression anastomosis process can be allowed to proceed over the ensuing days, resulting in the natural formation of an opening between the tissues. The fused magnets can either be allowed to expel naturally or the magnets can be retrieved in a follow-up surgical procedure. Alternatively, if immediate bypass is required, the tissues circumscribed by the magnets can be cut or perforated. Perforation can be accomplished with a variety of techniques, such as cautery, microscalpel, or balloon dilation of tissue following needle and guidewire access.

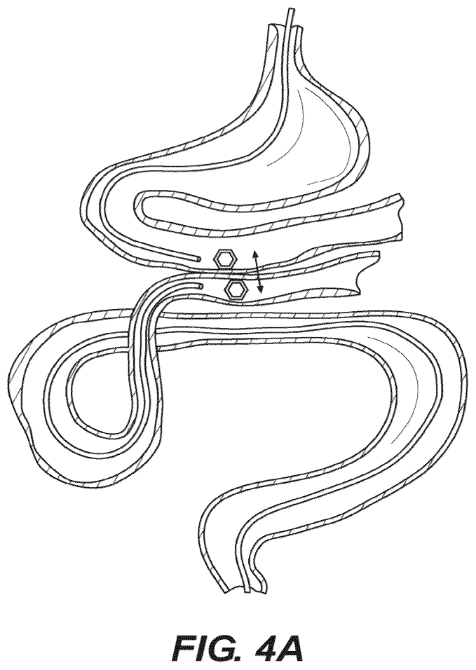

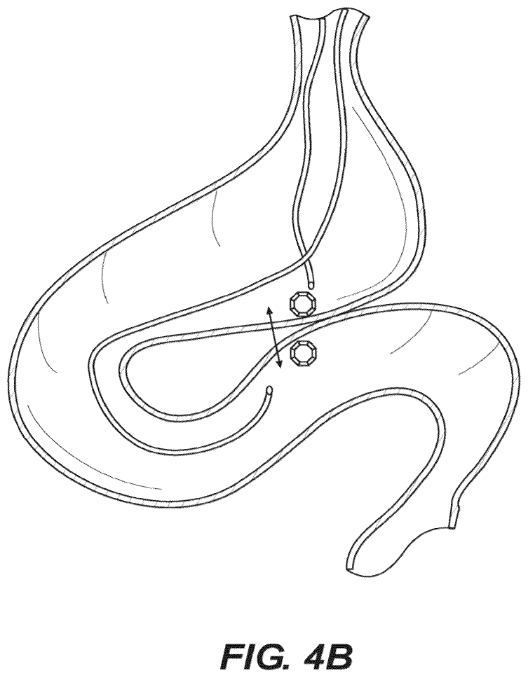

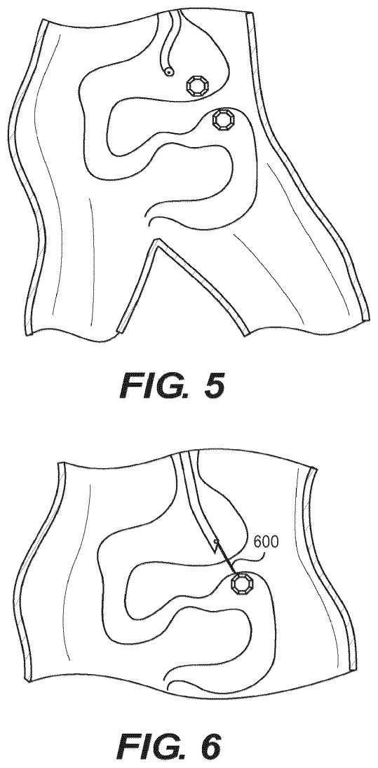

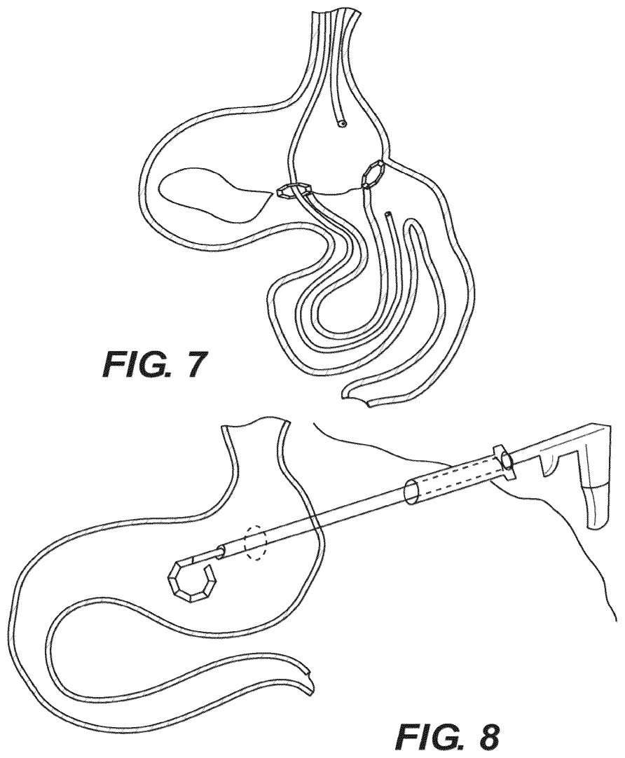

In some embodiments, the self-assembling magnetic devices are used to create a bypass in the gastrointestinal tract. Such bypasses can be used for the treatment of a cancerous obstruction, weight loss or bariatrics, or even treatment of diabetes and metabolic disease (i.e. metabolic surgery). Such a bypass could be created endoscopically, laparoscopically, or a combination of both. FIG. 3 illustrates the variety of gastrointestinal anastomotic targets that may be addressed with the devices of the invention: stomach to small intestine (A), stomach to large intestine (E), small intestine to small intestine (C), small intestine to large intestine (B), and large intestine to large intestine (D). It should be noted that additional anatomical targets that are not shown may be joined via anastomosis formation (e.g., esophagus to small intestine or jejunum, or large intestine or colon to rectum). In an endoscopic procedure, the self-assembling magnetic devices can be delivered using two simultaneous endoscopes, e.g., an upper endoscope or enteroscope residing in the upper small intestine, and a colonoscope residing in the lower small intestine, as shown in FIG. 4A. Alternatively, as shown in FIG. 4B, two simultaneous upper endoscopes (e.g., one residing in the stomach and the second in the small intestine) can be used to place the devices. In other embodiments, the self-assembling magnets can be delivered sequentially through the same endoscope, which has been moved between a first deployment position and a second deployment position. For example, in FIG. 4A, a single per-oral endoscope could deliver and deploy one self-assembling magnet in the small intestine, withdraw, and then deploy the second reciprocal magnet in the stomach. Again, magnet coupling could be confirmed using fluoroscopy. FIG. 5 illustrates removal of a single endoscope after placement of two magnetic devices.

A variety of techniques can be used to detect the first deployed magnetic device to assist placement of the second mating structure. Once the first device is deployed at the desired anastomotic location, the two deployed magnetic devices need to find one another's magnetic field so that they can mate and provide the compressional force needed to prompt formation of an anastomosis. Ideally, the devices can be roughly located within several cm of one another (e.g., using ultrasound), at which point the magnets should self-capture and self-align. Where this is not possible, other techniques such as one of the following techniques can be used. A first location technique involves a direct contact method using two endoscopes. Here an endoscope's displacement in an adjacent lumen creates a displacement seen by another endoscope in the adjacent lumen. The displacement identifies a potential intersection point for an anastomosis location. For example, a magnetic deployment tool (described below) will be deflected by the presence of a deployed device on the other side of a tissue wall.

The second location technique involves trans-illumination, whereby high intensity light from one endoscope is directed at the lumen wall of the proposed anastomosis site. Using this technique, another endoscope in the adjacent lumen looks for the light, which diffuses through the lumen wall and projects onto the wall of the adjacent lumen. This light represents the potential intersection anastomosis point. A cap or lens can also be placed over the light emitting endoscope to further intensify and pinpoint the proposed intersection point. A similar technique could use radio-wave- or ultrasound-transducers and receivers to collocate the endoscope tips. In some embodiments, a system may include an endoscope having a sensor and a magnetic anastomosis device for deployment using the endoscope.

A third location technique involves magnetic sensing techniques to determine the proximity of the deployed ring magnet in the adjacent lumen. By maximizing the magnetic field being sensed, the minimum distance between the adjacent channels can be identified. The magnetic sensor can be carried on a probe inserted down the working channel of the endoscope and utilize common magnetic sensing technology such as a Hall Effect Sensor or Reed switch.

With trans-illumination and magnetic sensing, an additional accessory may also assist with delivering magnetic devises to a precise anastomosis site. A radially expanding ring structure can be deployed with the endoscope or laparoscope that can press fit and seat itself on the scope's outer diameter. The outer diameter of this expanding element is sized to allow the deployed device to seat itself on this expanding element (again likely a press fit). With this expanding element and magnetic device radially seated about the endoscope axis, the endoscope can be directed to the ideal anastomotic location through direct contact, trans-illumination, or magnetic sensing, and then the mating magnet device released when the anastomosis site is identified.

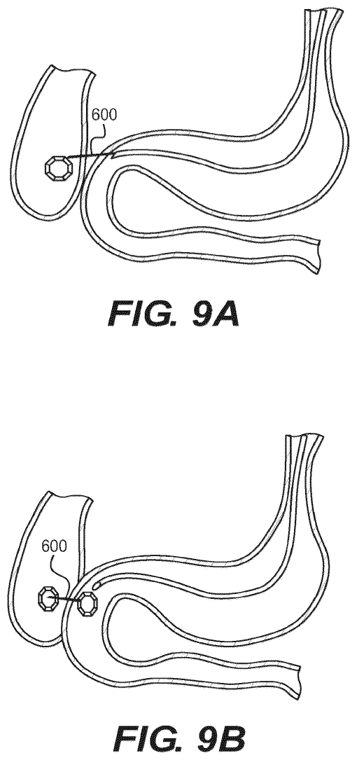

In other embodiments, the self-assembling magnet devices could be delivered using ultrasound guidance, e.g., endoscopic ultrasound. For example, using an echoendoscope in the stomach, a suitable small intestine target could be identified. As shown in FIG. 6, a delivery needle 600 (e.g., an aspiration needle) or catheter can be used to access to the small intestine target and deliver the self-assembling magnets into the small intestine lumen. The delivery can be guided with fluoroscopy or endoscopic ultrasound. Following self-assembly, these small intestine magnets would couple with a second set of magnets deployed in the stomach. The two devices can be delivered with the same needle or with different needles. It is also possible to deliver the first device with an endoscope and the second device with a needle or vice versa.

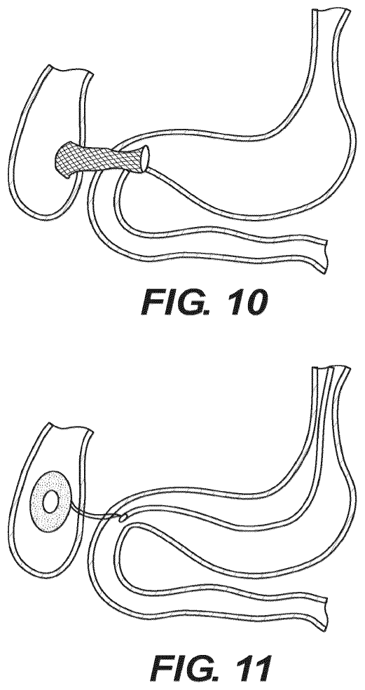

In another embodiment, illustrated in FIG. 7, a first anastomosis, created in an initial procedure, can be used to provide access for the creation of a second anastomosis. This process could theoretically be repeated multiple times to create additional anastomoses. For example, a gastrojejunal anastomosis (stomach to mid-small intestine) could serve as a conduit for the creation of a second, more distal gastrojejunal anastomosis. Ultimately, in this particular scenario, the stomach would have several bypasses to the small intestine. Additionally, in some instances, more anastomoses could be added to “titrate” to a specific clinical effect (e.g., lower glycosylated hemoglobin in type 2 diabetes). In alternative embodiments, an anastomosis may be placed to give access for a different type of surgery, e.g., tumor removal.

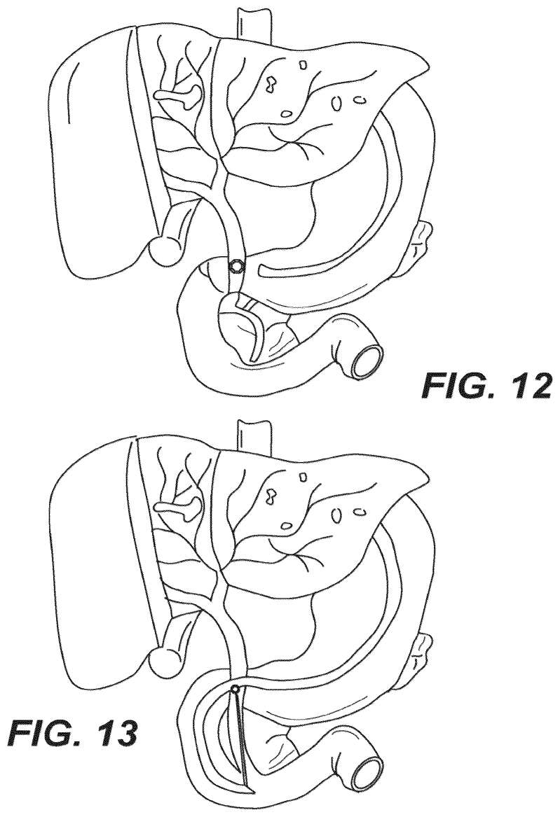

In another embodiment of delivery, the self-assembling magnets could be delivered laparoscopically through a surgical incision into the target organs (e.g., stomach, small intestine, large intestine, rectum, etc.) and allowed to couple to create an anastomosis, as shown in FIG. 8. Again, this procedure could be directed with fluoroscopy or ultrasound and the procedure can be purely laparoscopic, or a combination of endoscopic and/or laparoscopic and/or needle procedures.

Gastrointestinal anastomoses can be used to address a number of conditions. An anastomosis or series of anastomoses between the proximal bowel and distal bowel may be used for treatment of obesity and metabolic conditions, such as Type II diabetes and dyslipidemia. The procedure can also be used to induce weight loss and to improve metabolic profiles, e.g., lipid profiles. The bowel includes any segment of the alimentary canal extending from the pyloric sphincter of the stomach to the anus. In some embodiments, an anastomosis is formed to bypass diseased, mal-formed, or dysfunctional tissues. In some embodiments, an anastomosis is formed to alter the “normal” digestive process in an effort to diminish or prevent other diseases, such as diabetes, hypertension, autoimmune, or musculoskeletal disease.

Using the self-assembling magnetic devices as discussed herein, it is possible to create a side-to-side anastomosis that does not require exclusion of the intermediate tissues, as is common with state-of-the-art bariatric procedures. That is, using the devices of the invention (or other means for creating an anastomosis) it is possible to create an alternate pathway that is a partial bypass for fluids (e.g., gastric fluids) and nutrients (e.g., food), while at least a portion of the old pathway is maintained. This design allows the ratio of “normal” to “modified” digestion to be tuned based upon the goals of the procedure. In other words, using the described procedure, a doctor can choose the ratio of food/fluids shunted down the new (partial) bypass versus food/fluids shunted down the old pathway. In most instances, the fraction shunted down the bypass limb will drive the patient toward the desired clinical endpoint (e.g., weight loss, improvement in glycosylated hemoglobin, improvement in lipid profile, etc.) The mechanism by which the endpoints are achieved may involve early macronutrient delivery to the ileum with stimulation of L-cells and increase in GLP-1 production, for example. The mechanism may also involve loss of efficiency of nutrient absorption, especially glucose, thereby reducing blood glucose levels. At the same time, however, the fraction shunted down the old pathway protects against known metabolic complications that can be associated with bariatric surgery such as excessive weight loss, malabsorptive diarrhea, electrolyte derangements, malnutrition, etc.

To achieve a desired ratio of bypass (e.g., re-routing food and secretions to flow down the new pathway, say, 70% or 80% or 90% or 100% of the time), the size, location, and possibly number of anastomoses will be important. For example, for a gastrojejunal anastomosis, it may be critical to place the anastomosis in a dependent fashion to take advantage of the effects of gravity. Also, instead of a round anastomosis, it may be better to create a long, oval-shaped anastomosis to maximize anastomotic size. Alternatively, multiple gastrojejunal anastomoses may be used to titrate to a certain clinical endpoint (e.g., glycosylated hemoglobin in Type II diabetes). Most of the procedures described herein may be used to place one or more anastomoses, as needed, to achieve the desired clinical endpoint. For example, the two endoscope procedures illustrated in FIGS. 4A and 4B can be used to create a partial bypass of a portion of the bowel. Based upon the desired ratio of bypassed and non-bypassed nutrients, the anastomoses shown in FIGS. 4A and 4B can be made larger, e.g., greater than 1 cm in open diameter, or several smaller anastomoses can be placed to achieve the desired ratio.

The procedure is also adjustable. For example, a first anastomosis may be formed and then, based upon clinical tests performed after the procedure, one or more anastomoses can be added to improve the results of the clinical tests. Based upon later clinical results, it may be necessary to add yet another anastomosis. Alternatively, it is possible to partially reverse the condition by closing one or more anastomosis. Because the partially bypassed tissues were not removed, they can return to near normal functionality with the passage of greater amounts of nutrients, etc. The anastomoses may be closed with clips, sutures, staples, etc. In other embodiments, a plug may be placed in one or more anastomoses to limit the ratio of nutrients that traverse the “normal” pathway. Furthermore, it is possible to close an anastomosis in one location in the bowel and then place a new anastomosis at a different location. Thus, is possible to generally and tunably create partial bypasses, or a series of partial bypasses, between portions of the bowel to achieve clinical endpoints, e.g., as described in FIG. 3.

The described procedures may also be used with procedures that remove or block the bypassed tissues, as is common with bariatric procedures. For example, a gastrojejunal anastomosis may be coupled with a pyloric plug (gastric obstruction) or another closure of the pylorus (e.g., sutured closure) to shunt food completely down the new bypass. Such procedures can be used, for example, to bypass tissue that is diseased, e.g., because of cancer.

In another category of procedures, endoscopic ultrasound (EUS) can be used to facilitate guided transgastric or transduodenal access into the gallbladder for placement of a self-assembling magnetic anastomosis device. Once gallbladder access is obtained, various strategies can be employed to maintain a patent portal between the stomach and the gallbladder or the duodenum and the gallbladder. In another embodiment, gallstones can be endoscopically retrieved and fluid drained. For example, using the described methods, an anastomosis can be created between the gallbladder and the stomach. Once the gallbladder is accessed in a transgastric or transduodenal fashion, the gallstones can be removed. Furthermore, the gallbladder mucosa can be ablated using any number of modalities, including but not limited to argon plasma coagulation (APC), photodynamic therapy (PDT), sclerosant (e.g., ethanolamine or ethanol).

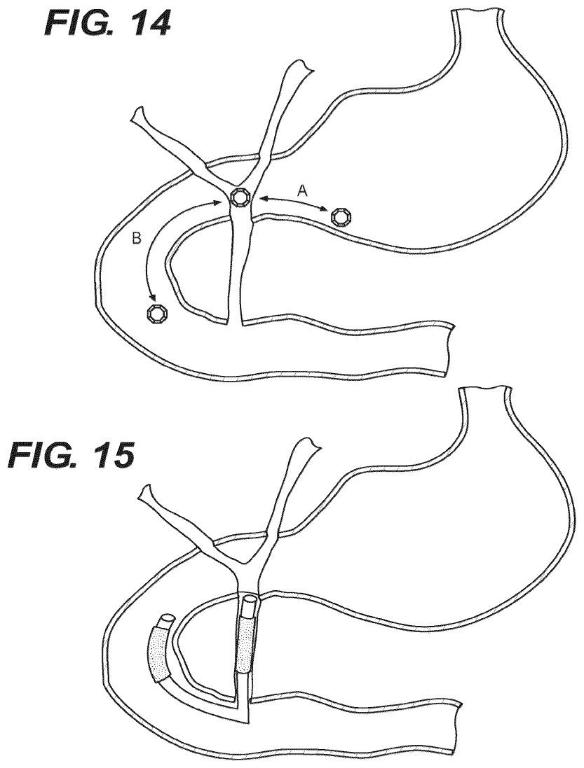

One strategy for creation of a portal is to deploy self-assembling magnets via an endoscopic needle under ultrasound guidance into the gallbladder and also into the stomach or duodenum. These magnets will mate and form a compression anastomosis or fistula. A second strategy for creation of a portal is to deploy self-assembling magnets via an endoscopic needle 600 as shown in FIGS. 9A and 9B. While the coupled magnetic assemblies are shown as octagons, the closed frame could take the shape of any polygonal structure, e.g., a square, a circle, a triangle, hexagon, heptagon, nonagon, decagon, dodecagon, etc. One such device would be deployed into the gallbladder, and the mating device would be deployed into the stomach or duodenum. In the same fashion as discussed above with respect to gastrointestinal deployment, the tissue circumscribed by the two magnetic devices can be cut with cautery, microscalpel, needle-knife, or other deployable cutting mechanism. In another embodiment, the coupled tissues can be left to necrose and form the anastomosis.

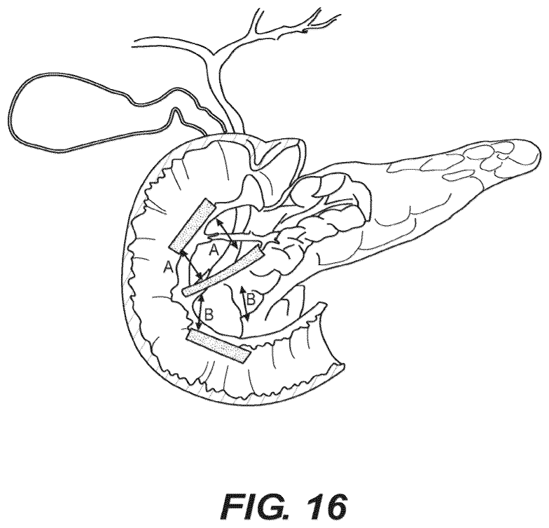

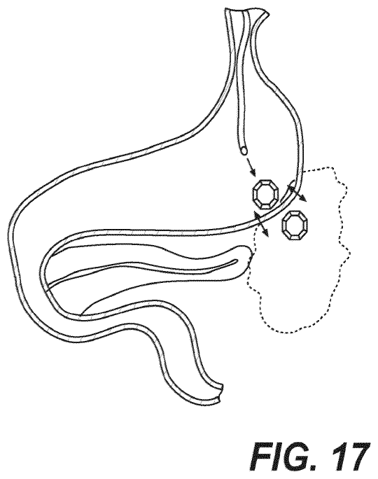

The devices need not be limited to forming holes, however. Other structures can be coupled to one or more mating magnetic devices to created additional functionality. For example, a stent could be deployed between tissues, such as the gallbladder and the stomach, as shown in FIG. 10. Alternatively, the gallbladder magnet could be coupled to a balloon-based device that fills with air, fluid, magnetic pieces or magnetic particles. Upon inflation, the balloon would serve as an anchor in the bile duct following placement. The balloon could also have an annular configuration to allow for immediate access after coupling with the second magnet. See, e.g., FIG. 11. Regardless of embodiment, however, it is critical to contain the original access pathway within the confines of the coupled magnets, i.e., not leaving a pathway for the escape of bile. Otherwise, the opening will allow bile leakage that can result in peritonitis.

Another medical application for self-assembling magnets is direct biliary access. Currently, to achieve decompression for a malignant biliary stricture, endoscopic retrograde cholangiopancreatography (ERCP) is performed. The biliary tract is accessed endoscopically through the papilla in retrograde fashion and a stent is deployed across the stricture over a guidewire. These stents frequently require subsequent procedures for exchange, clean-out, or placement of additional overlapping stents. The need for exchange and cleaning is required to counteract the high rate of infection of the biliary tree (i.e., cholangitis) when using an ERCP procedure. Because of the high rate of morbidity, ERCP is typically limited to patients that have no other option to address pancreatic disease.

Using devices of the invention, however, it is possible to easily form an anastomosis between the bile duct (preferably the main bile duct) and either the duodenum or the stomach (choledocho-gastric and choledocho-duodenal anastomoses, respectively). This anastomosis is permanent and typically does not require intervention if located apart from the diseased tissue. In an embodiment, a biliary magnetic device is delivered directly into the bile duct under endoscopic ultrasound guidance. As described below, the self-assembling magnetic device is extruded through a needle or catheter, whereupon it deploys in the correct configuration. Using fluoroscopy or ultrasound, it is then possible to confirm that the device has self-assembled and is in the correct location. In some embodiments, the magnetic device may be tethered to the delivery needle or catheter by means of a detachable wire or suture to enable mechanical retraction until optimal positioning is confirmed.

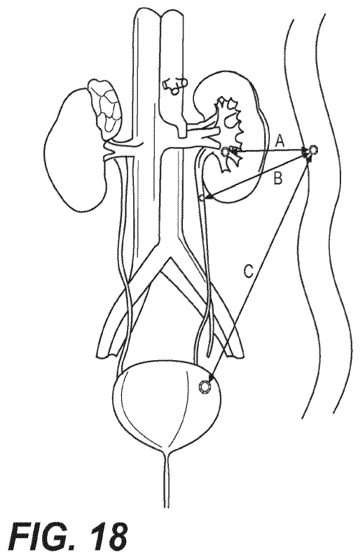

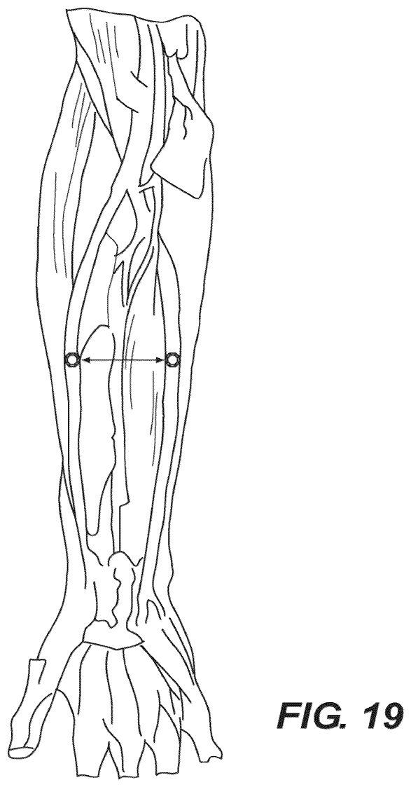

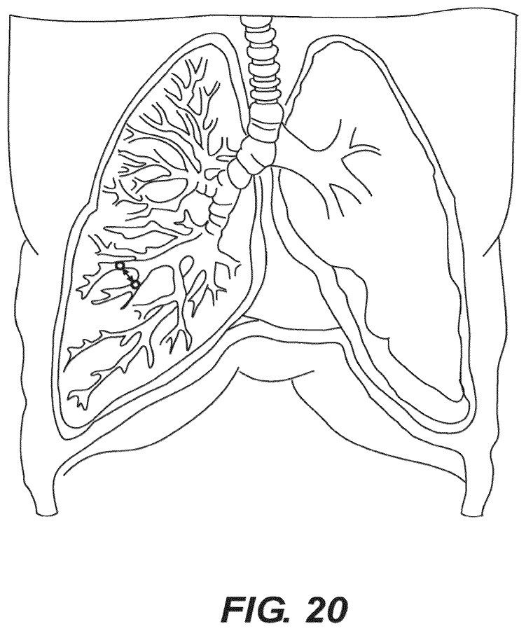

In one embodiment, the magnetic device can be delivered endoscopically to the bile duct via wall of the duodenum, as shown in FIG. 12. In another embodiment, the biliary magnet can be delivered in conventional retrograde fashion through the ampulla into the bile duct, as shown in FIG. 13. One benefit of retrograde delivery is that it avoids needle punctures across tissue planes, as is the case with the deployment method shown in FIG. 12. Regardless of the method for delivering the biliary magnets, however, a second magnetic device is required in either the gastric (A) or duodenal (B) lumen, as shown in FIG. 14. Typically, this decision is dependent upon the patient's anatomy (e.g., size of the duodenal lumen) and the location of the initial biliary magnet. In scenarios based on endoscopic ultrasound needle delivery, the second magnetic device can be connected to the biliary magnet via the aforementioned detachable wire, and therefore extruded through the same delivery needle/catheter. Alternatively, the second device can be pre-attached to the exterior of the endoscope and slid into position for coupling after biliary magnet deployment. The latter procedure may be more applicable to forward-viewing echoendoscopes but may be used with endoscopes, generally.

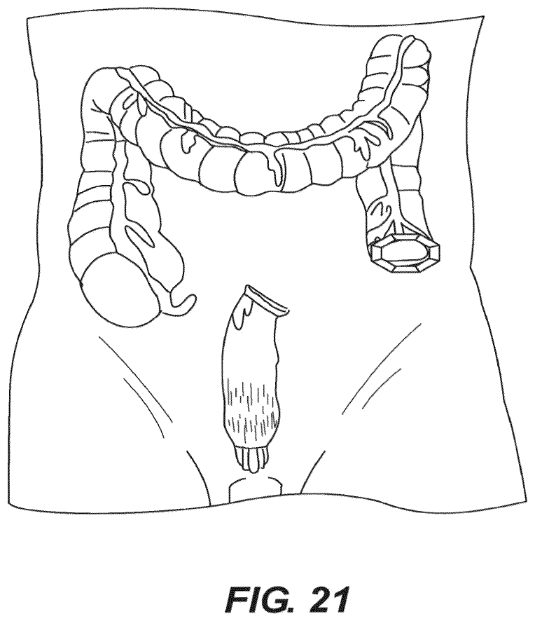

In another embodiment, the biliary magnet is a balloon-based device that fills with air, fluid, magnetic pieces or magnetic particles, similar to previously described with respect to gallbladder procedures. Upon inflation, the balloon would serve as an anchor in the bile duct following placement. In an embodiment, the balloon could have an annular configuration to allow for immediate access after coupling with the second magnet. Additionally, like the gallbladder procedures described above, a biliary magnetic device can be used with a stent form-factor. In an embodiment, the stent has an internal biliary magnet and a hinged external magnet. The stent can be inserted in retrograde fashion through the ampulla into the bile duct. The hinged external magnet can then be swung around and coupled with the internal biliary magnet to form a fistula between the bile duct and the duodenum, as shown in FIG. 15.

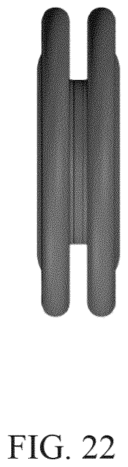

The magnetic devices of the invention can also be used to treat pancreatic diseases. For example, the pancreatic duct requires decompression in certain disease states, such as chronic pancreatitis. Currently, extensive pancreatic duct decompression requires surgery (e.g., Peustow surgery in which the pancreas is filleted along the axis of the pancreatic duct and connected to a loop of small intestine for improved pancreatic drainage). As an alternative to Peustow surgery, extensive pancreatic duct decompression can be accomplished via creation of a large magnetic compression anastomosis between the pancreatic duct and either the stomach or duodenum using a magnetic pancreatic catheter, as shown in FIG. 16. The catheter can be magnetic along its entire length or only at certain intervals. The catheter can be in the form of a stent or straw. The pancreatic duct can be accessed using conventional ERCP methods (retrograde cannulation through the ampulla) or by direct needle access using endoscopic ultrasound (EUS). The magnetic pancreatic catheter can be delivered into the pancreatic duct and coupled with a second magnetic device in either the stomach or duodenum. As in the biliary scenario described above, the magnetic pancreatic catheter could be hinged to the second magnetic device.

The magnetic devices of the invention can also be used to form anastomoses between the large intestine or colon and the rectum, e.g., following removal of some or all of the colon such as for treatment of colorectal cancer, inflammatory bowel disease, or diverticular disease.

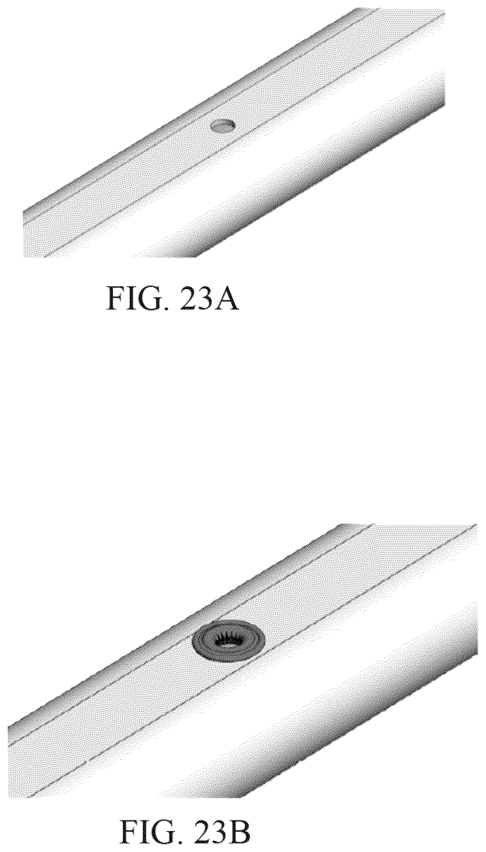

Self-assembling magnetic devices can also be used to access and drain fluid collections located adjacent to the gastrointestinal tract, as shown in FIG. 17. For example, following a bout of pancreatitis, pancreatic fluid collections can form that require drainage. While drainage can be accomplished using surgery or a percutaneous catheter, endoscopic drainage has been found to be more clinically and cost-effective, but can be complicated by bleeding, perforation, and/or inadequate drainage. As an alternative to surgical draining, magnetic devices of the invention can be delivered through a needle or sharpened catheter into the collection under endoscopic ultrasound (EUS) guidance, as shown in FIG. 17. Following assembly, the first magnetic device is coupled to the second magnetic device that has been placed in the gastrointestinal lumen (e.g. stomach). In order to speed removal after drainage, the first magnet may be tethered by a connecting wire as previously described. As described previously, the intervening tissue can be cut using electrocautery or dilation followed by needle and wire access. Additional devices, such as magnetic coupling clamps can be used to control blood flows to allow for “blood-less” endoscopic entry into the collection.

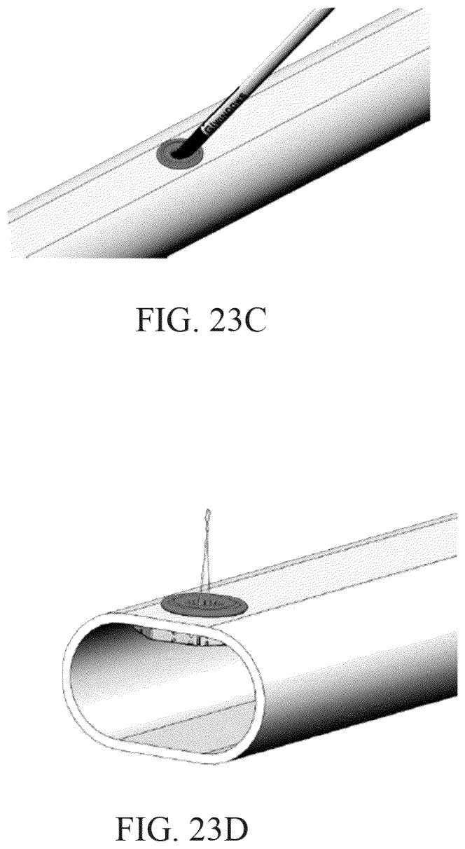

Self-assembling magnets can also be used for urological applications such as forming bypasses to treat an obstructed urogenital tract, as shown in FIG. 18. For example, a magnetic anastomosis could be created between the renal calyx and bowel (A), between the ureter and bowel (B), or between the bladder and bowel (C). Self-assembling magnetic devices of the invention can be delivered into the urological tract using an endoscope, laparoscope, or needle, as described above. The reciprocal magnetic device could be delivered into the gastrointestinal tract using an endoscope, laparoscope, or needle as previously described. In other embodiments, the devices can be used for reproductive procedures, such as bypassing a portion of obstructed fallopian tube or bypassing a vasectomy.

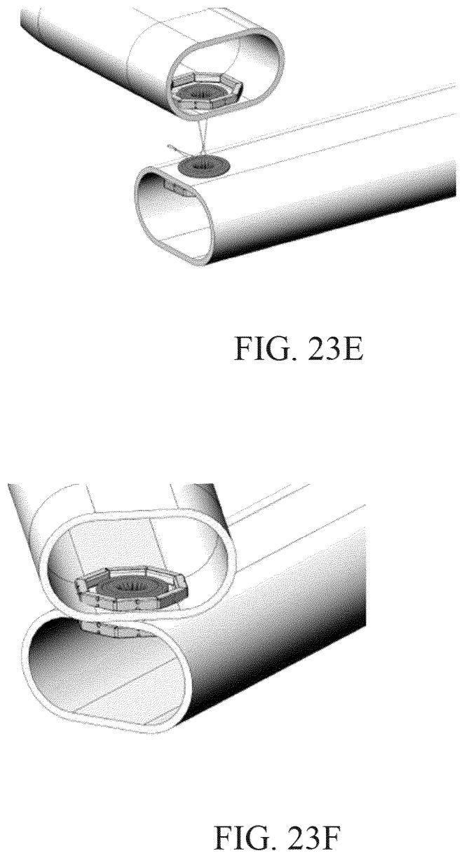

In yet another application, self-assembling magnetic devices can be used to create vascular anastomoses or to treat cardiac conditions. For example, a magnetic anastomosis coupling can be formed between adjacent blood vessels with magnetic devices, as shown in FIG. 19. In an embodiment, the self-assembling devices can be delivered with a vascular delivery device, such as a catheter. Additionally, as described above with respect to gallbladder and pancreatic applications, a shunt can be installed to bypass a portion of the vasculature that is weak or blocked.

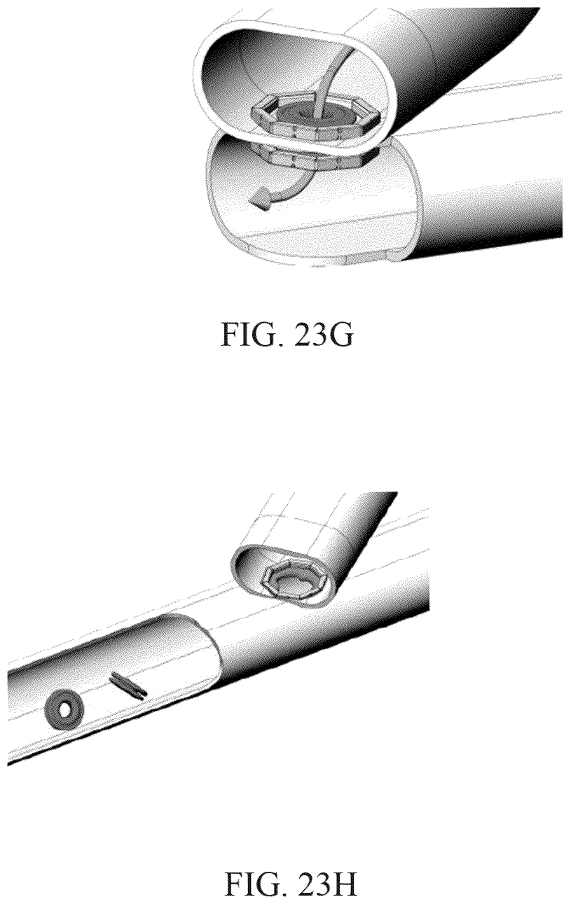

Self-assembling magnets can also be used for pulmonary applications such as forming bypasses in the airway to treat chronic obstructive pulmonary disease (COPD). For example, magnetic anastomoses can be created by deploying self-assembling magnetic devices into adjacent bronchioles, as shown in FIG. 20. Creation of pulmonary “bypasses” could lower airway resistance that characterizes respiratory diseases such as COPD.

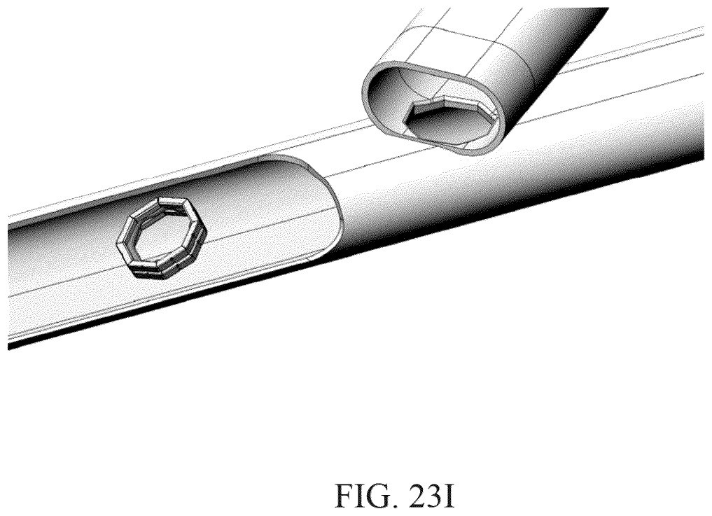

Self-assembling magnetic devices can also be used to create surgical stomas for diversion of a fecal stream, e.g., into a colostomy bag. For example, a magnetic anastomosis can be created by deploying self-assembling magnets into the gastrointestinal tract (e.g. large intestine), as shown in FIG. 21, and then coupling the interior magnet to an external magnet worn and secured at the level of the skin. The exterior magnetic device may be coupled to yet a third magnetic device that is coupled to a collection device. Such a system allows easy removal of the collection device for cleaning, etc.

Certain embodiments described below are specifically configured for use with self-assembly magnetic compression anastomosis devices of the types described in commonly-owned U.S. patent application Ser. No. 18/229,988 entitled MAGNETIC COMPRESSION ANASTOMOSIS DEVICES WITH MULTIPIECE INTERNAL VERTEBRAE SUPPORT STRUCTURES filed Aug. 3, 2023 and in commonly-owned U.S. patent application Ser. No. 18/230,066 entitled MAGNETIC COMPRESSION ANASTOMOSIS DEVICE WITH MULTIPIECE VERTEBRA filed Aug. 3, 2023, each of which is hereby incorporated by reference in its entirety. For convenience, such devices may be referred to as Flexagon™ magnetic compression anastomosis devices. It should be noted, however, that embodiments described below are not necessarily limited to use with such Flexagon™ magnetic compression anastomosis devices.

In some cases, it is necessary or desirable to support an acute otomy before or after delivery of a magnetic compression anastomosis device or other device or for another purpose such as maintaining or enlarging the otomy such as to allow for fluid flow through the otomy or to allow procedures to be performed through the otomy. Certain embodiments include systems and methods for preserving and manipulating acute otomies using the types of otomy control devices described below and in commonly-owned U.S. Provisional Patent Application No. 63/419,509 entitled SYSTEMS AND METHODS FOR PRESERVING AND MANIPULATING OF ACUTE OTOMIES filed Oct. 26, 2022 and in commonly-owned U.S. Provisional Patent Application No. 63/435,724 entitled SYSTEMS AND METHODS FOR PRESERVING AND MANIPULATING OF ACUTE OTOMIES filed Dec. 28, 2022, each of which is hereby incorporated herein by reference in its entirety. For convenience, such devices may be referred to as OTOLoc™ otomy control devices. It should be noted, however, that embodiments described below are not necessarily limited to use with such OTOLoc™ otomy control devices.

Generally speaking, the described otomy control devices secure a periphery and/or adjacent tissue of a target otomy site and maintain the size of an otomy through a single wall of a vessel, organ, or lumen within the body. Among other things, this can reduce unintentional trauma, dilation, contraction, or locomotion of an otomy during a surgical procedure and also can allow immediate communication between lumens before the complete creation of the permanent anastomosis.

As used herein particularly with regard to an otomy control device, the terms “distal” and “proximal” are generally relative to an access or delivery device, e.g., with the “distal” being further from the access or delivery device than the “proximal.” In some situations, the terms “distal” and “proximal” may be relative to a lumen, e.g., with the “distal” generally being within the lumen and the “proximal” generally being outside of the lumen.

It should be noted that certain embodiments are described herein with reference to a nitinol shape-memory material, although it should be noted that embodiments are not limited to nitinol but instead other shape-memory materials may be used in various alternative embodiments.

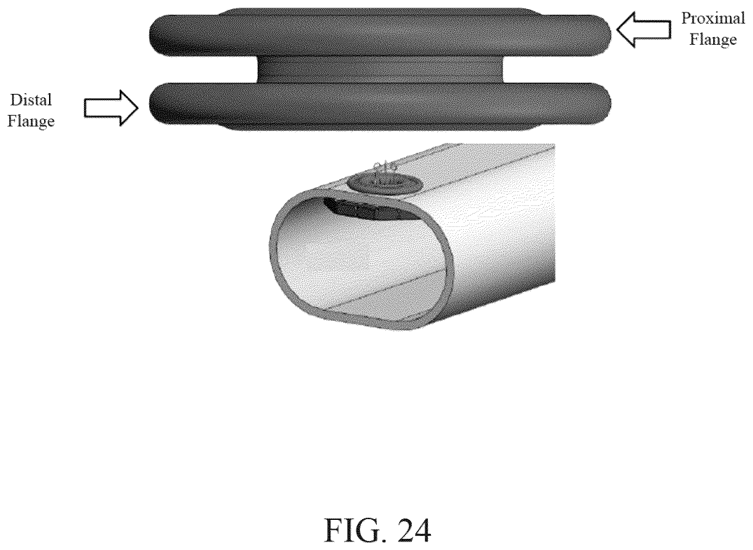

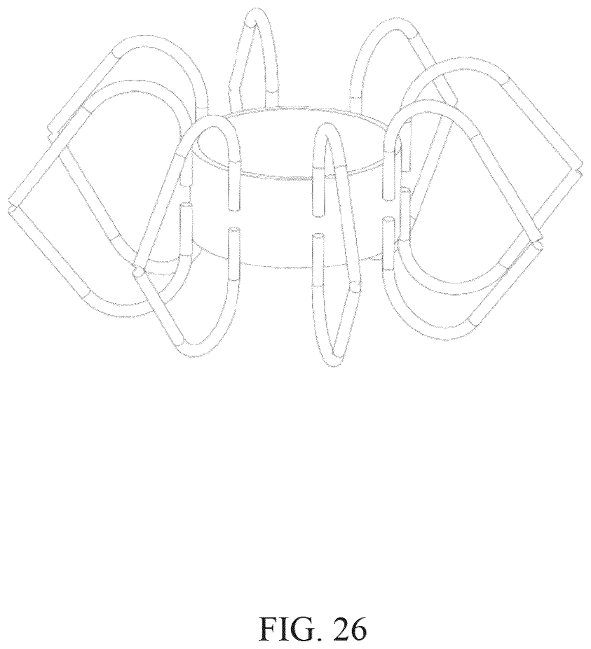

FIG. 22 shows a side-view of an exemplary otomy control apparatus in accordance with certain embodiments. This particular embodiment comprises a dual-flange style apparatus (referred to herein as a grommet). As shown in FIG. 22 and FIG. 24, a grommet apparatus may include a distal flange and a proximal flange connected by a channel of smaller diameter than the flanges. The central channel between the flanges, as shown in FIG. 22, connects the flanges while capturing and controlling the size of the otomy.

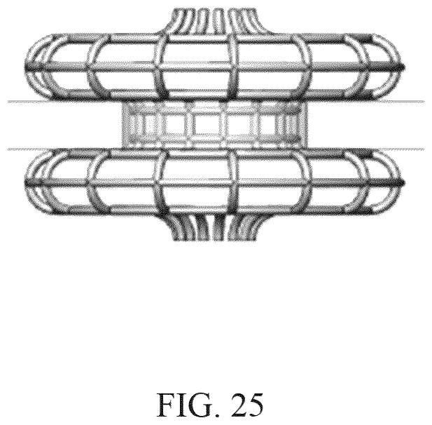

A grommet has an outer diameter and an inner diameter, the inner diameter defining a channel between the flanges allowing for fluidic passage through the grommet when deployed in an otomy (as shown in FIG. 23B and to be described herein). The front view of the grommet (FIG. 23B) comprises an annular shape with an inner diameter defining a channel connecting the flanges, allowing for capture and control of the otomy as the flanges engage the tissue surrounding the otomy.

FIGS. 23A-23I show a method of controlling an otomy during the creation of a permanent anastomosis. The procedure for controlling an otomy and anastomosis formation may be completed endoscopically, laparoscopically, robotically, by way of open-field surgery, or a combination thereof.

An otomy is formed in an organ and/or bowel as shown in FIG. 23A. After otomy formation, an access device cannulates the otomy, entering the distal side of the tissue into the organ/bowel lumen.

A grommet apparatus is compressed within an access device in a delivery configuration, with the distal flange toward the distal end of the access device, and the proximal flange toward the proximal end of the access device. The access device may house the grommet within a rigid tube such as a cannula, or via a flexible tube such as a catheter or endoscope. The grommet may, in some embodiments, be housed within a secondary delivery device within the access device. The secondary delivery device may move proximally, distally, and rotationally within the access device. The secondary delivery device may house the grommet prior to grommet deployment into the body of a patient.

The access device may, in some embodiments, comprise a pusher device, in addition to or in replacement of the secondary delivery device, for deploying the grommet into the lumen of a patient. The pusher may include a monolithically formed pushrod or wire, cable, or articulating mechanism the advances the grommet from the access device. The pusher may be rigid, semi-rigid, or flexible.

The medical professional advances the access device into the distal lumen and deploys the distal flange of the apparatus, with the proximal flange remaining inside the access device. The distal flange may be deployed by pulling back on the access device, advancing the secondary delivery device, and/or advancing the pusher. After the distal flange is deployed into the lumen, on the distal side of the otomy, the distal flange expands into the deployed configuration.

The medical professional pulls back on the access device into the body cavity from the lumen. The medical professional then pulls back on the access device, pulls back on the secondary delivery device, and/or advances the pusher to deploy the proximal flange on the proximal side of the tissue surrounding the otomy. Upon deploying from the access device, the proximal flange expands into the deployed configuration. As shown in FIG. 23B, the grommet is then in the fully deployed configuration, with the distal flange engaging the distal side of the tissue surrounding the otomy within the lumen, and the proximal flange engaging the proximal side of the tissue surrounding the otomy.

In some embodiments, the medical professional may insert the access device endoscopically into a lumen. From inside the lumen, the medical professional advances the access device through the tissue wall into the body cavity. The distal flange may be deployed by pulling back on the access device, advancing the secondary delivery device, and/or advancing the pusher. After the distal flange is deployed into the body cavity, on the distal side of the otomy, the distal flange expands into the deployed configuration.

The medical professional pulls back on the access device into the lumen from the body cavity. The medical professional then pulls back on the access device, pulls back on the secondary delivery device, and/or advances the pusher to deploy the proximal flange on the proximal side of the tissue surrounding the otomy. Upon deploying from the access device, the proximal flange expands into the deployed configuration. As shown in FIG. 23B, the grommet is then in the fully deployed configuration, with the distal flange engaging the distal side of the tissue surrounding the otomy within the body cavity, and the proximal flange engaging the proximal side of the tissue surrounding the otomy within the lumen.

In the fully deployed configuration, as shown in FIG. 23B, the grommet allows for control of the otomy, reduction tissue dilation/tearing around the otomy, and a channel for immediate fluidic flow through the otomy.

After the grommet is deployed within the otomy, the medical professional may advance an access device through the otomy for further surgical procedures, as shown in FIG. 23C.

In the creation of an anastomosis, the medical professional may deploy a magnetic compression anastomosis device or other device into the lumen of the organ/bowel of a patient at a target anastomosis site through the grommet. Additionally, or alternatively, the medical professional may deploy a magnetic compression anastomosis device or other device into the lumen prior to deployment of the grommet. FIG. 23D shows a magnetic compression anastomosis device deployed in the lumen behind the grommet. Whether the magnetic compression anastomosis device is deployed before or after the grommet, the diameter of the magnetic compression anastomosis device is greater than the diameter of the grommet and therefore is unable to pass through the grommet out of the lumen.

The methods and steps as described above and depicted in FIGS. 23A-23D may be repeated in another organ/bowel/lumen of the patient at another target anastomosis site one or more times. The medical professional then can bring the otomies of the one or more target sites in proximity with each other. The otomies may be manipulated, for example, endoscopically, laparoscopically, robotically, or in open field surgery. The medical professional may manipulate the otomies, for example, using sutures attached to the magnetic compression anastomosis devices, as is shown in FIG. 23E.

After the magnetic compression anastomosis devices are in proximity with each other, they mate due to attractive magnetic forces as shown in FIG. 23F, compressing the tissue therebetween to form an anastomosis. As is shown in FIG. 23G, the channel within the grommets allows for immediate patency and fluidic passage between the lumens before the complete creation of an anastomosis.

The magnetic compression anastomosis devices compress and necrose the tissue therebetween. As the tissue necroses, the grommets may fall away from the target site and pass naturally from the patient, as is shown in FIG. 23H. After complete formation of the anastomosis, the magnetic compression anastomosis devices also fall away from the target site and pass naturally from the patient, as is shown in FIG. 23I, leaving a fully formed anastomosis.

Compression anastomosis devices described herein may include articulating magnetic compression anastomosis devices.

The magnetic anastomosis devices of certain embodiments generally comprise magnetic segments that can assume a delivery conformation and a deployed configuration. The delivery configuration is typically linear so that the device can be delivered to a tissue via a laparoscopic “keyhole” incision or with delivery via a natural pathway, e.g., via the esophagus, with an endoscope or similar device. Additionally, the delivery conformation is typically somewhat flexible so that the device can be guided through various curves in the body. Once the device is delivered, the device will assume a deployed configuration of the desired shape and size by converting from the delivery configuration to the deployed configuration automatically. The self-conversion from the delivery configuration to the deployment configuration is directed by coupling structures that cause the magnetic segments to move in the desired way without intervention.

In general, a magnetic anastomosis procedure involves placing a first and a second magnetic structure adjacent to first and second portions of tissues, respectively, thus causing the tissues to come together. Once the two devices are brought into proximity, the magnetic structures mate and bring the tissues together. With time, an anastomosis of the size and shape of the devices will form and the devices will fall away from the tissue. In particular, the tissues circumscribed by the devices will be allowed to necrose and degrade, providing an opening between the tissues.

FIG. 24 depicts a side-view of the exemplary grommet otomy control apparatus of FIG. 22. An exemplary grommet has a proximal flange and a distal flange connected by a channel therebetween. The diameter of the channel is less than the diameter of the flanges. As is shown in FIG. 24, the grommet is deployed across the otomy, with the one flange engaging the intralumenal side of the tissue and the other flange engaging the extralumenal side of the tissue. The inner diameter channel allows for immediate patency and fluidic passage through the otomy. Additionally, the passage allows for deployment of a compression anastomosis device, as shown in FIG. 24, within the lumen on the distal side of the otomy.

In various embodiments, magnets may be incorporated into the grommet, combining the grommet and magnetic compression anastomosis device into a single apparatus. The magnet or magnets may be incorporated into the distal and/or proximal flange, or the entire grommet. This would allow for the grommet to control the otomy, while also acting as the magnetic compression anastomosis device.

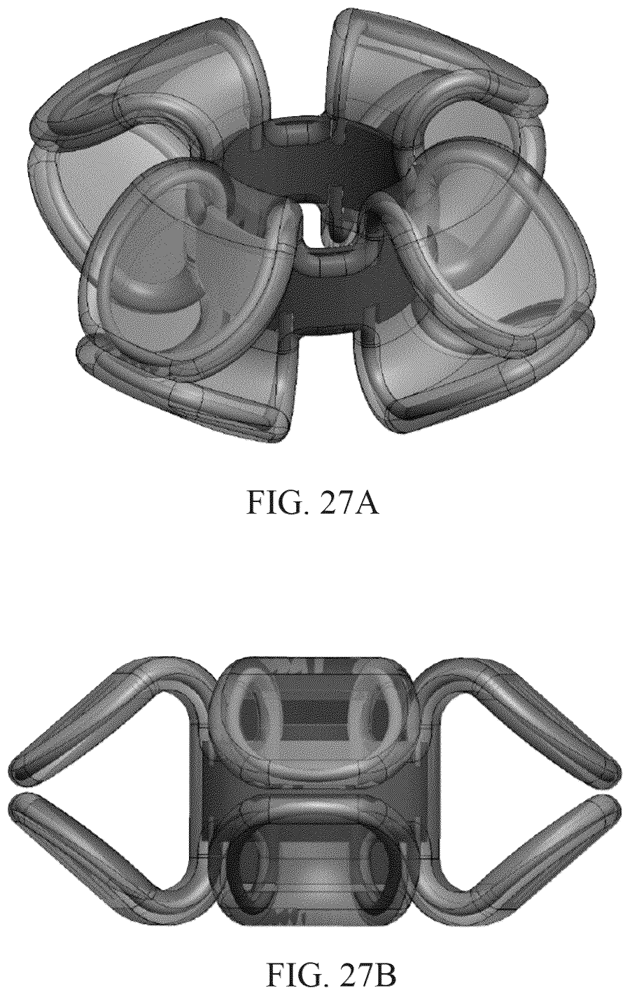

FIG. 25 depicts an exemplary embodiment of a grommet-style otomy control apparatus. The grommet depicted in FIG. 25 comprises a semi-rigid or rigid conduit that acts as an open channel to facilitate deployment and alignment of surgical tools as well as fluidic passage through the otomy. The grommet may be constructed from nitinol, stainless steel, or other bio-compatible materials.

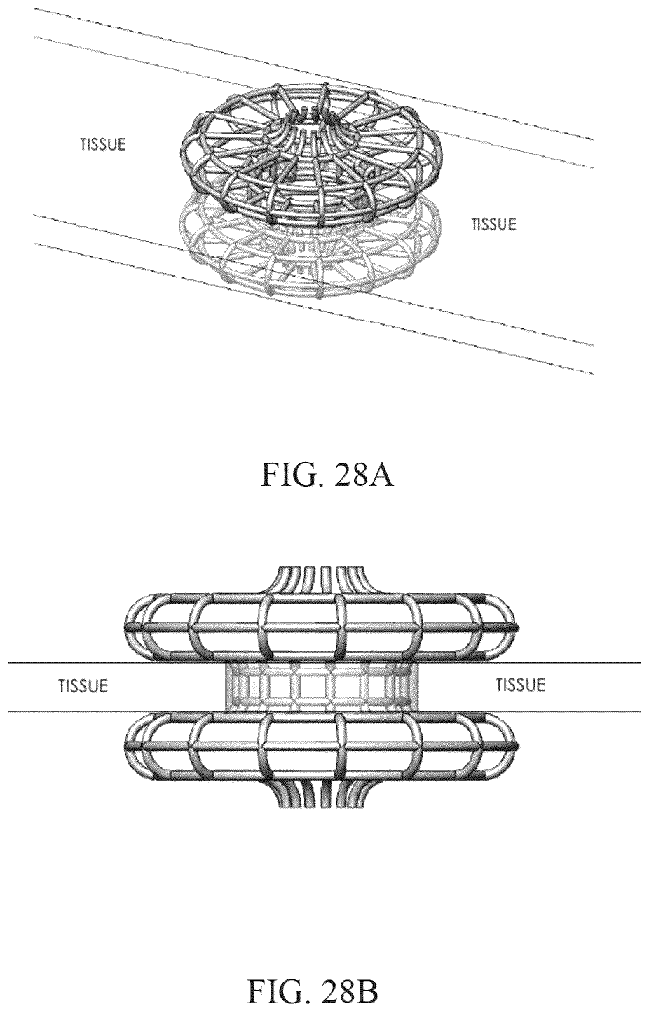

FIG. 26 depicts a nitinol tubing or wire hybrid grommet. Nitinol wires or tubes may be attached to a central tube section to clamp a periphery of tissue surrounding an otomy from both sides. The central tube section allows for passage of fluid and surgical tools through the otomy while preserving the shape and size and reducing tearing of the tissue surrounding the otomy. The central tube section can be made of any appropriate material, e.g., nitinol, stainless steel, etc. In order to deliver this device into a patient, the device would be stored inside a delivery device such as an endoscope, laparoscope, catheter, etc. with the top set of nitinol wires/tubes extended upward and with the bottom set of nitinol wires/tubes extended downward. Note that the embodiments shown in FIGS. 26-27B, 30A-36B, and 38A-42B may be delivered into the patient in this manner.

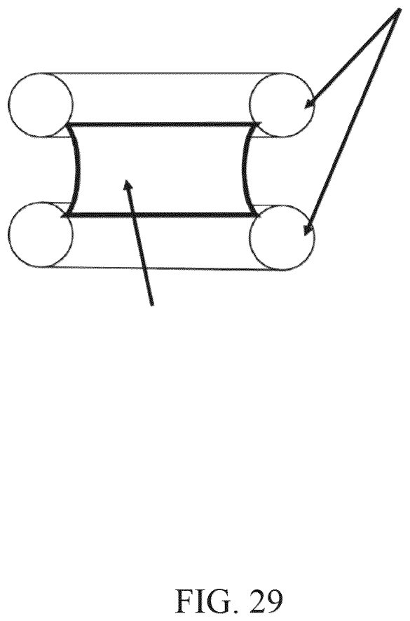

FIGS. 27A-27B depict another exemplary nitinol tubing or wire hybrid grommet. The grommet may comprise two or more bent wire loops attached to a tube section. FIG. 4 depicts a wire hybrid grommet comprising four bent wire loops. The wire loops may be constructed from nitinol, stainless steel, or other bio-compatible materials. The bent wire loops clamp the tissue surrounding the otomy, securing the size and shape of the otomy and reducing tissue tearing. The tube section allows for the passage of fluid or surgical tools through the otomy.

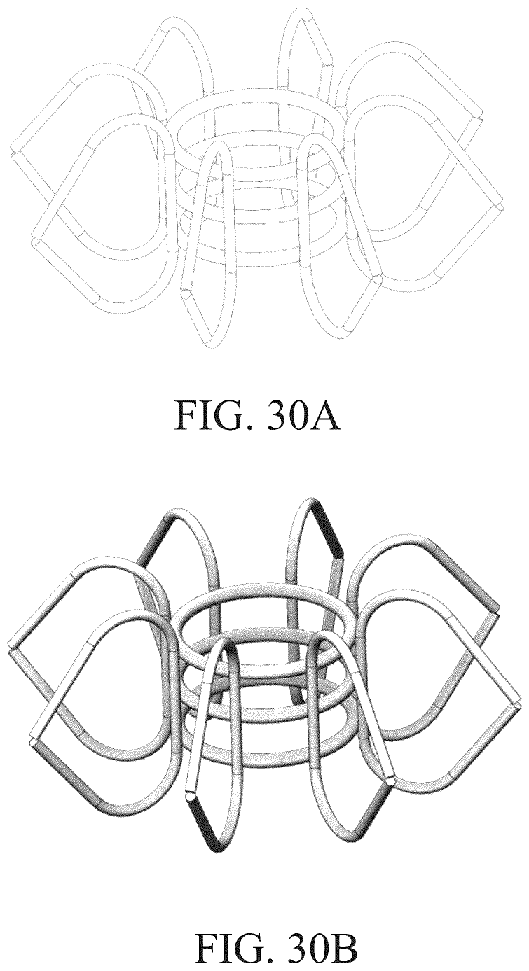

FIGS. 28A-28B depict nitinol wire stent array grommets. The nitinol wire stent array clamps the tissue surrounding an otomy site, securing the otomy and reducing tissue deformation around the otomy site. As is depicted in FIG. 28B, the flanges of the nitinol wire stent array engage the distal and proximal sides of the tissue respectively, while the tube section passes through the otomy, allowing for immediate patency and fluid passage before complete formation of the permanent anastomosis. The tube section also maintains the diameter of the otomy as the flanges reduce tearing and dilation of the tissue surrounding the otomy during a surgical procedure.

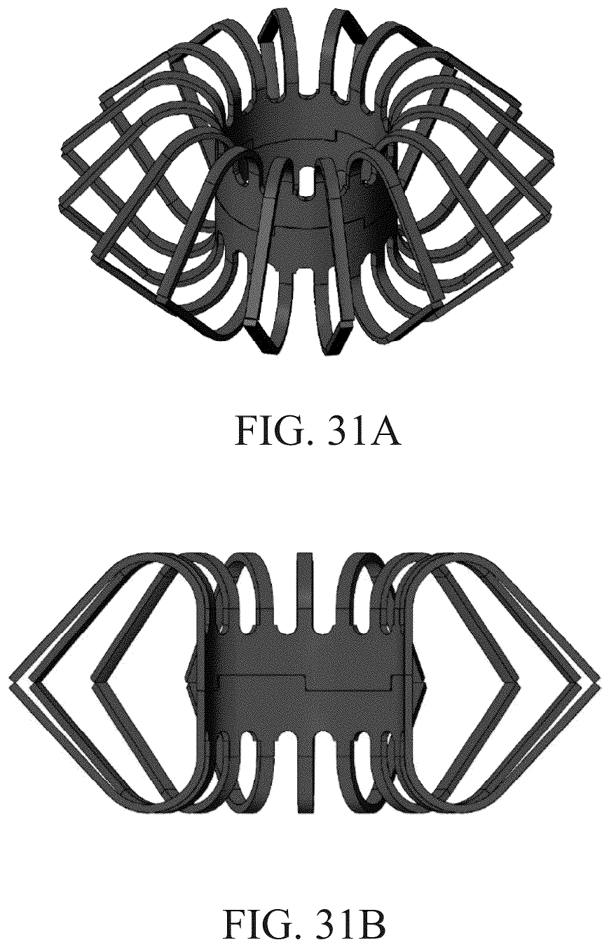

FIG. 29 depicts an exemplary embodiment of an inflatable grommet. An inflatable grommet is deflated in a delivery configuration within an access device. After deployment into the distal lumen of an otomy, the distal flange of the grommet is inflated to a deployed diameter. The proximal flange is then deployed on the proximal side of the otomy and inflated to a deployed diameter. Once inflated into the deployed configuration, the inflatable grommet secures the otomy and reduces tearing of tissue around the otomy site.

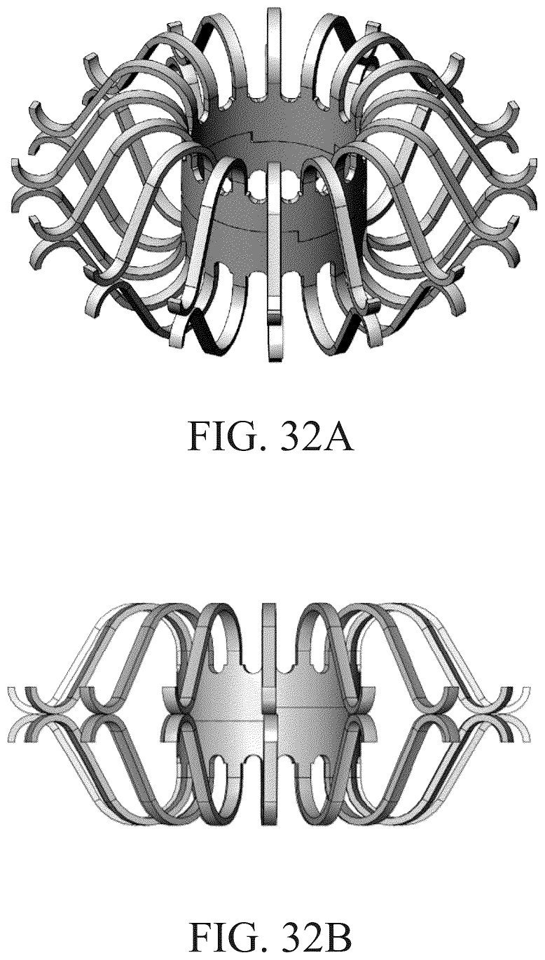

FIGS. 30A-30B depict perspective views of an exemplary nitinol tubing/wire hybrid grommet for securing an acute otomy. This device is similar to the device shown in FIG. 26 but includes a central open ring section rather than a central tube section to which the nitinol wires or tubes are attached, forming a nitinol wire or tubing array that clamps a periphery of tissue surrounding an otomy site from both sides. The array has bent tube or wire sections to clamp tissue, while the hollow center section allows for immediate patency through the otomy and fluid passage before complete formation of the permanent anastomosis. The center section also maintains the diameter of the otomy as the nitinol wires/tubing reduces tearing and dilation of the tissue surrounding the otomy during a surgical procedure. The central open ring section can be made of any appropriate material, e.g., nitinol, stainless steel, etc. This device can be delivered by storing the device in a delivery device as described above with reference to FIG. 26.

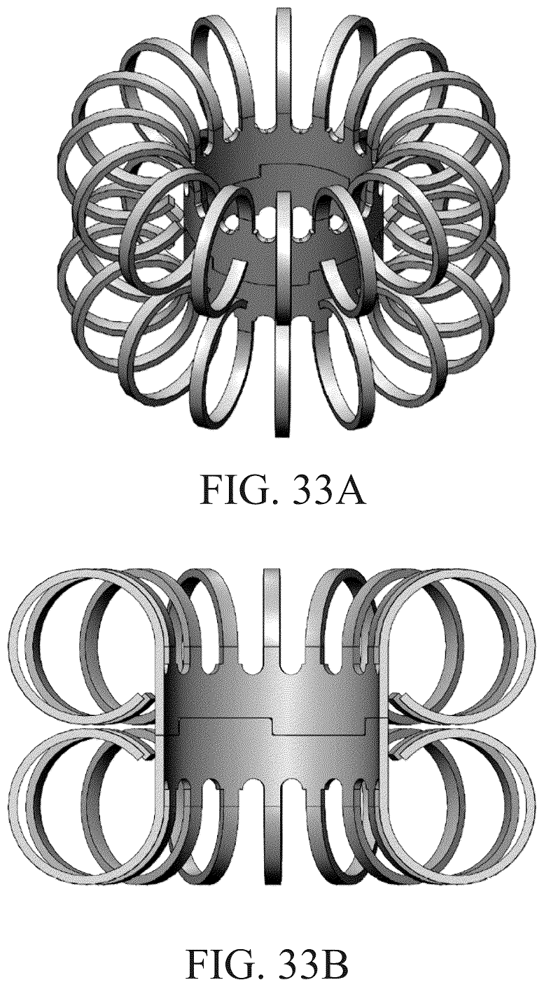

FIGS. 31A-31B depict a two-piece nitinol tubing/wire hybrid grommet. Nitinol wires may be attached to a center section to clamp a periphery of tissue surrounding an otomy. The two halves of the nitinol tubing/wire array grommet are stored in a delivery device in a delivery configuration. The medical professional advances the delivery device through a created otomy, and pulls back on the delivery device to deploy the distal half of the nitinol tubing/wire array grommet into a deployed configuration. The medical professional may then pull the delivery device further back to deploy the proximal half of the grommet into a deployed configuration. The outer arms of the two halves clamp the periphery tissue of the created otomy, while the center portion allows for passage of fluid and surgical tools through the otomy. This preserves the shape and size and reducing tearing of the tissue surrounding the otomy. The two halves of the array grommet may be held together mechanically, magnetically, and/or by other mechanism. As depicted in FIGS. 31A-31B, the two halves have corresponding mating elements to better ensure that the two halves join in a desired configuration (which in this embodiment aligns corresponding arms of the two halves).

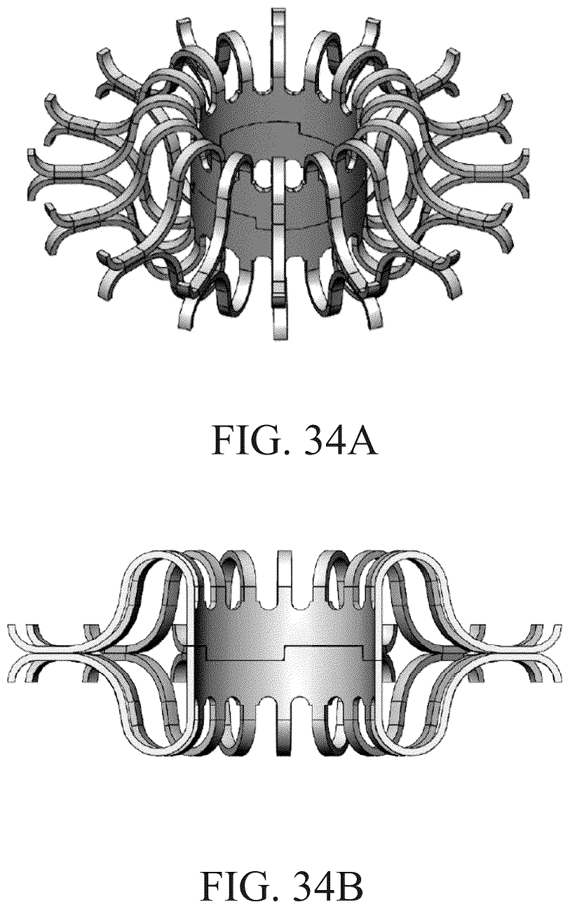

FIGS. 32A-32B depict an exemplary two-piece nitinol tubing/wire grommet. Nitinol wires may be attached to a center section to clamp a periphery of tissue surrounding an otomy. The center portion allows for passage of fluid and surgical tools through the otomy while preserving the shape and size and reducing tearing of the tissue surrounding the otomy. The two halves of the array grommet may be held together mechanically, magnetically, and/or by other mechanism. As depicted in FIGS. 32A-32B, the two halves have corresponding mating elements to better ensure that the two halves join in a desired configuration (which in this embodiment aligns corresponding arms of the two halves).

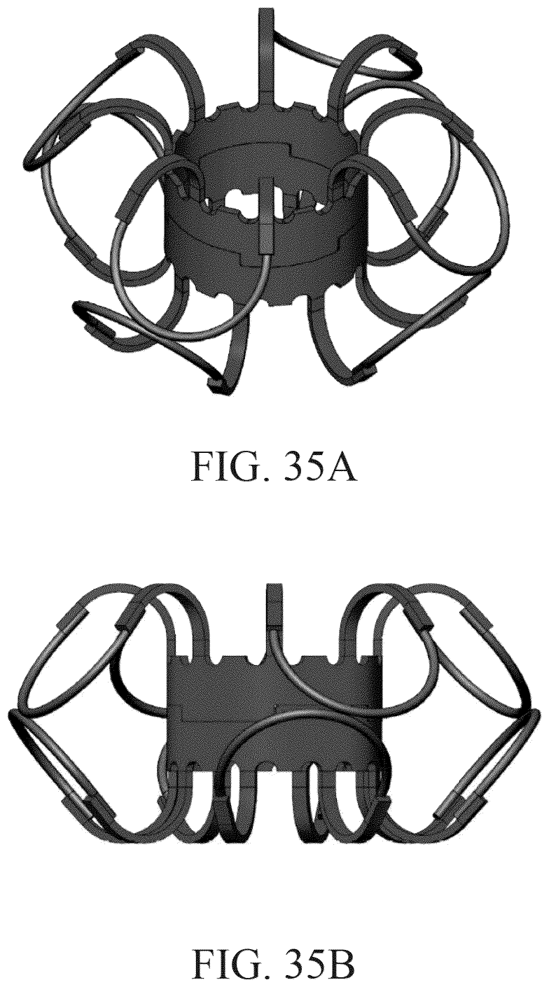

FIGS. 33A-33B depict perspective views of an exemplary two-piece nitinol tubing/wire array grommet. Nitinol tubing/wire arms may be attached to the circumference of a center section of the grommet. The arms clasp the tissue surrounding a created otomy while the center section allows for immediate passage of fluids and other materials such as surgical tools through the otomy before the creation of a permanent anastomosis. This secures the tissue surrounding the otomy site, reducing tearing, dilation, or locomotion of the otomy site.

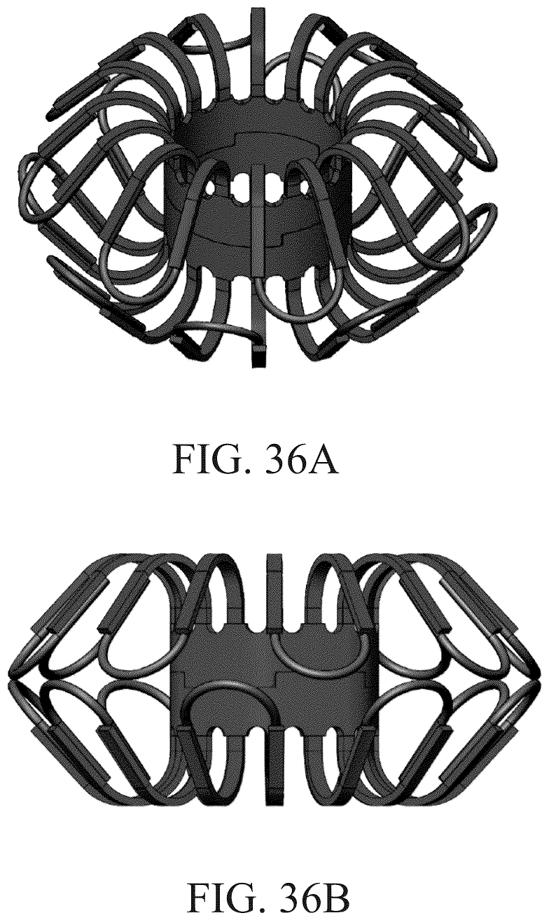

FIGS. 34A-34B depict another embodiment of a two-piece nitinol tubing/wire array grommet. Nitinol tubing/wire arms may be attached to the circumference of a center section of the grommet. The arms clasp the tissue surrounding a created otomy while the center section allows for immediate passage of fluids and other materials such as surgical tools through the otomy before the creation of a permanent anastomosis. This secures the tissue surrounding the otomy site, reducing tearing, dilation, or locomotion of the otomy site.

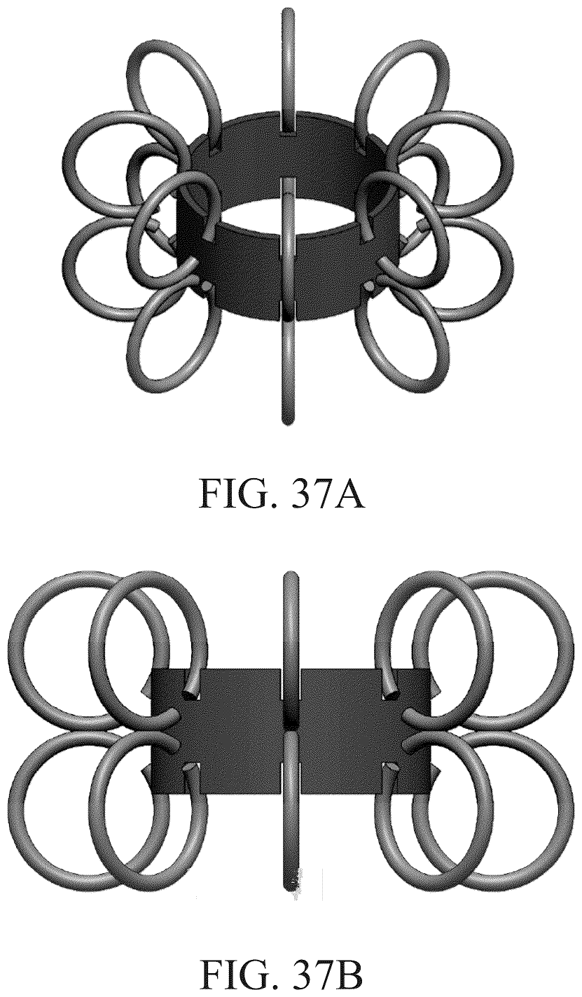

FIGS. 35A-35B depict perspective views of another embodiment of a two-piece nitinol tubing/wire array grommet. The grommet may comprise two or more bent wire loops attached to a tube section. FIGS. 35A-35B depict a wire hybrid grommet comprising eight bent wire loops. The wire loops may be constructed from nitinol, stainless steel, or other bio-compatible materials. The bent wire loops clamp the tissue surrounding the otomy, securing the size and shape of the otomy and reducing tissue tearing. The tube section allows for the passage of fluid or surgical tools through the otomy.

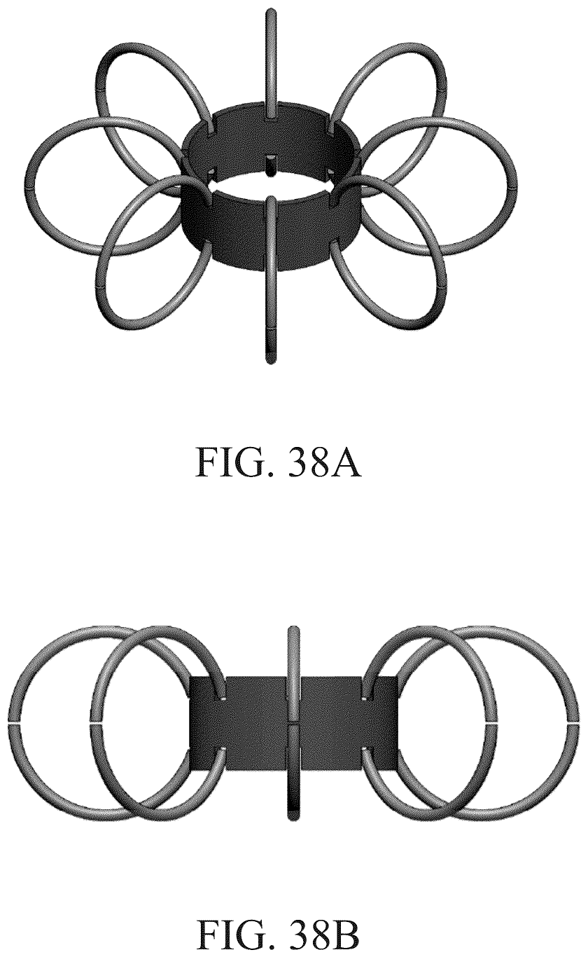

FIGS. 36A-36B depict perspective views of another embodiment of a two-piece nitinol tubing/wire array grommet. The grommet may comprise two or more bent wire loops attached to a tube section. FIGS. 36A-36B depict a wire hybrid grommet comprising sixteen bent wire loops. The wire loops may be constructed from nitinol, stainless steel, or other bio-compatible materials. The bent wire loops clamp the tissue surrounding the otomy, securing the size and shape of the otomy and reducing tissue tearing. The tube section allows for the passage of fluid or surgical tools through the otomy.

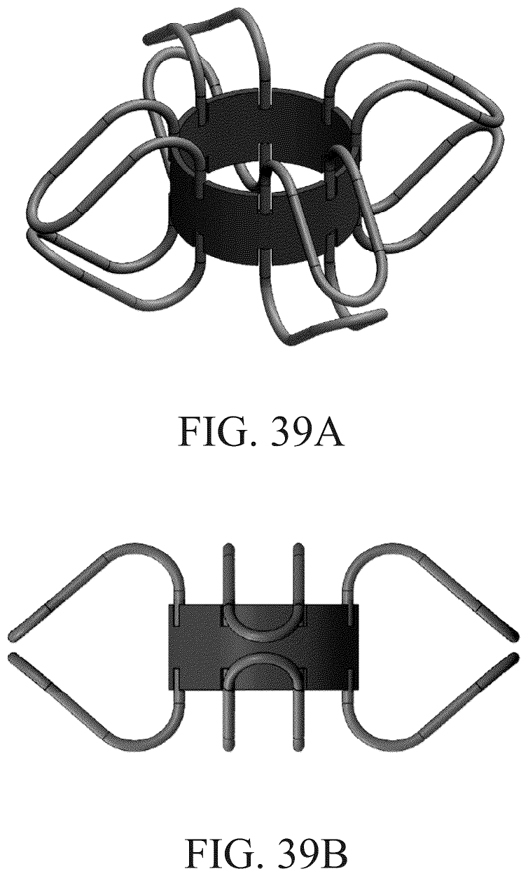

FIGS. 37A-37B depict perspective views of another embodiment of a nitinol tubing/wire array grommet. The grommet of FIGS. 37A-37B may comprise two or more bent wire loops. FIGS. 37A-37B depict a grommet comprising sixteen wire loops, eight on the distal half of the grommet, and eight on the proximal half. The wire loops may be constructed from nitinol, stainless steel, or other bio-compatible materials. The bent wire loops clamp the tissue surrounding the otomy, with the circumference of the loops perpendicular to the tissue surface. This secures the size and shape of the otomy and reduces tissue tearing. The center section of the grommet allows for the passage of fluid or surgical tools through the otomy.

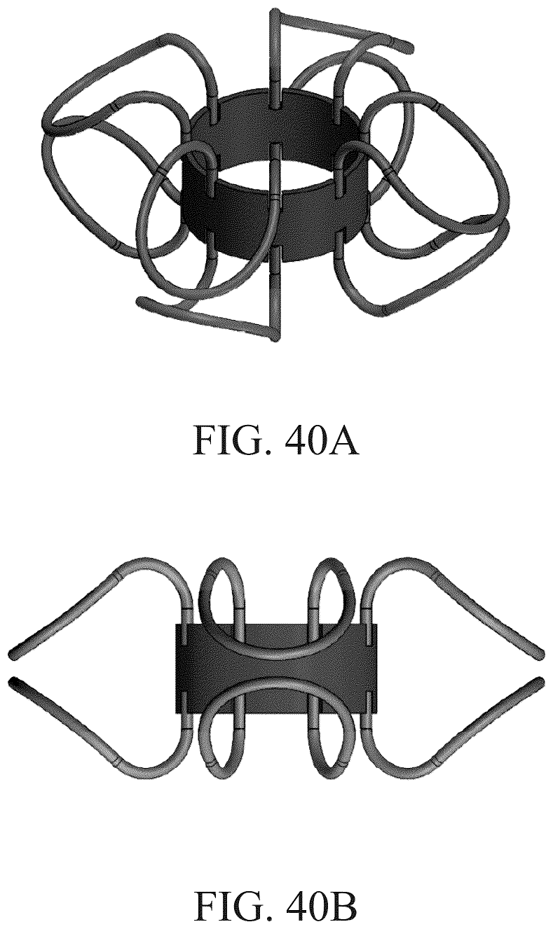

FIGS. 38A-38B depict an embodiment of a nitinol tubing/wire array grommet comprising wire half loops. The grommet of FIGS. 38A-38B comprise sixteen wire half-loops, with eight half loops on the proximal half of the device, eight half loops on the distal half of the device, and a hollow center section. The grommet device is stored in a delivery device in a delivery configuration. The medical professional advances the delivery device through a created otomy. Once on the distal side of the otomy, the medical professional pulls back on the delivery device to deploy the distal half of the grommet on the distal side of the tissue surrounding the otomy into a deployed configuration. The medical professional may pull further back on the delivery device through to the proximal side of the otomy to deploy the proximal half of the grommet device on the proximal side of the tissue surrounding the otomy into a deployed configuration. Once in the deployed configuration, the half loops of the distal half come into proximity with the half loops of the proximal half of the device to clasp the tissue surrounding the otomy. This secures the size and shape of the otomy and reduces tissue tearing. The center section of the grommet allows for the passage of fluid or surgical tools through the otomy.

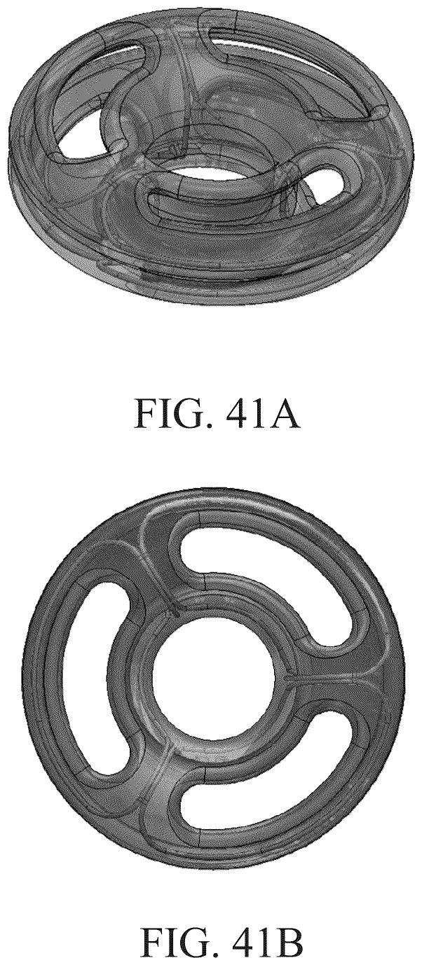

FIGS. 39A-39B depict perspective views of another embodiment of a nitinol tubing/wire array grommet. The grommet of FIGS. 39A-39B may comprise two or more bent wire loops. FIGS. 39A-39B depict a grommet comprising eight wire loops, four on the distal half of the grommet, and four on the proximal half. The wire loops may be constructed from nitinol, stainless steel, or other bio-compatible materials. The bent wire loops clamp the tissue surrounding the otomy, with the circumference of the loops perpendicular to the tissue surface. This secures the size and shape of the otomy and reduces tissue tearing. The center section of the grommet allows for the passage of fluid or surgical tools through the otomy.

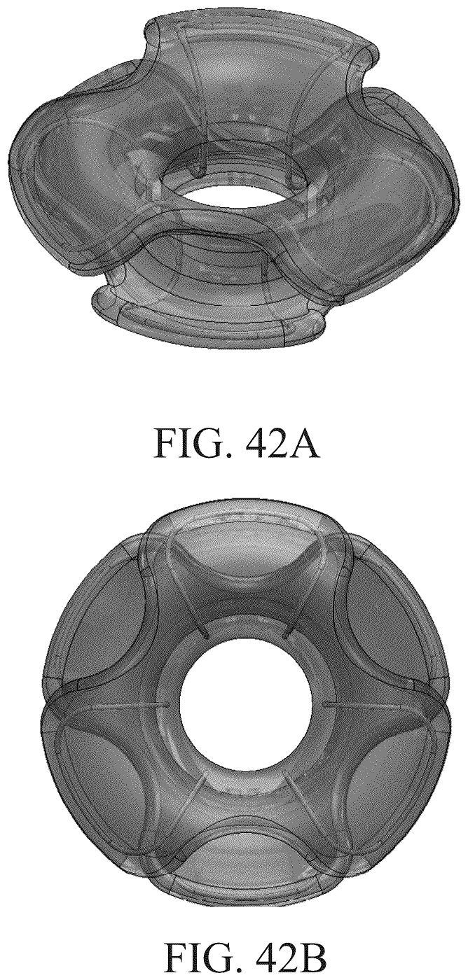

FIGS. 40A-40B depict perspective views of another embodiment of a nitinol tubing/wire array grommet. The grommet of FIGS. 40A-40B may comprise two or more bent wire loops. FIGS. 40A-40B depict a grommet comprising eight wire loops, four on the distal half of the grommet, and four on the proximal half. The wire loops may be constructed from nitinol, stainless steel, or other bio-compatible materials. The bent wire loops clamp the tissue surrounding the otomy, with the circumference of the loops perpendicular to the tissue surface. This secures the size and shape of the otomy and reduces tissue tearing. The center section of the grommet allows for the passage of fluid or surgical tools through the otomy.

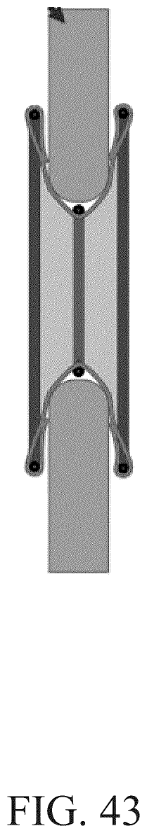

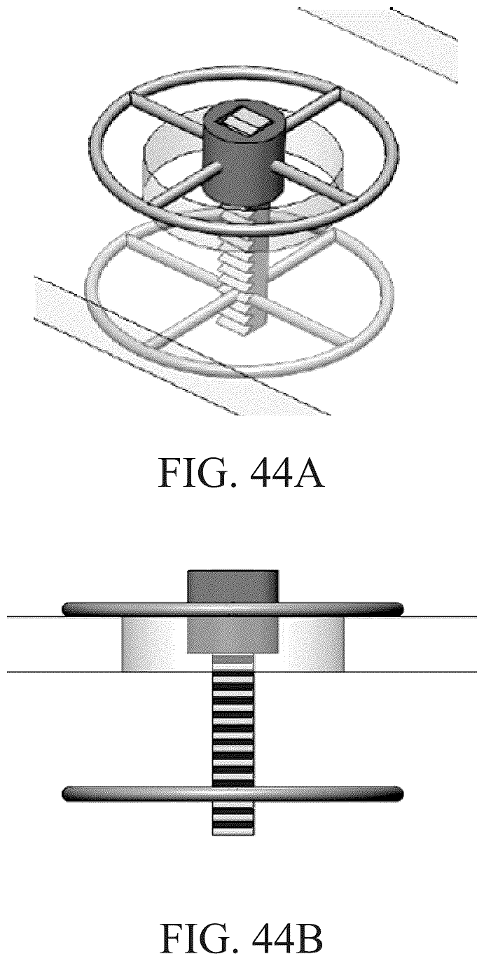

FIGS. 41A-41B and FIGS. 42A-42B depict an embodiment of a nitinol tubing/wire hybrid grommet. The grommet may comprise two or more wire loops enveloped in a biocompatible material, creating a distal flange and a proximal flange. The flanges may be continuous as shown in FIGS. 42A-42B or may comprise cutaway sections as shown in FIGS. 41A-41B. A hollow center section connecting the two flanges may be present. The grommet device is stored in a delivery device in a delivery configuration. The medical professional advances the delivery device through a created otomy. Once on the distal side of the otomy, the medical professional pulls back on the delivery device to deploy the distal flange of the grommet into the distal side of the tissue surrounding the otomy into a deployed configuration. The medical professional may pull further back on the delivery device through to the proximal side of the otomy to deploy the proximal flange of the grommet device in the proximal side of the tissue surrounding the otomy into a deployed configuration. Once in the deployed configuration, the flanges clasp the tissue surrounding the otomy. This secures the size and shape of the otomy and reduces tissue tearing. The center section of the grommet allows for the passage of fluid or surgical tools through the otomy.

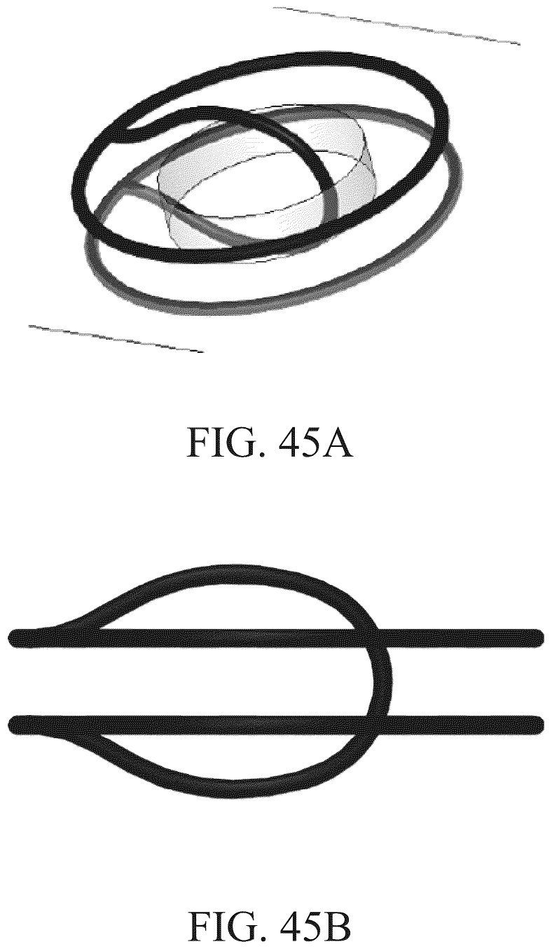

FIG. 43 depicts a triple sleeved hoop device for securing tissue surrounding a created otomy. The otomy sleeve comprises a small, central hoop with two larger hoops parallel to the central hoop, one on each side. The small hoop provides a work path/conduit centered on the otomy for fluid and/or surgical tools to pass through the otomy. The larger hoops tension a sleeve and prevent the device from being pulled out of the otomy. The sleeved hoop is stored in a delivery device in a delivery configuration. The medical professional advances the delivery device through a created otomy. Once on the distal side of the otomy, the medical professional pulls back on the delivery device to deploy the distal larger hoop of the triple sleeved hoop into the distal side of the tissue surrounding the otomy into a deployed configuration. The medical professional may pull further back on the delivery device through to the proximal side of the otomy to deploy the proximal larger hoop of the triple sleeved hoop in the proximal side of the tissue surrounding the otomy into a deployed configuration. Once in the deployed configuration, the larger hoops clasp the tissue surrounding the otomy. This secures the size and shape of the otomy and reduces tissue tearing. The central hoop allows for the passage of fluid or surgical tools through the otomy.

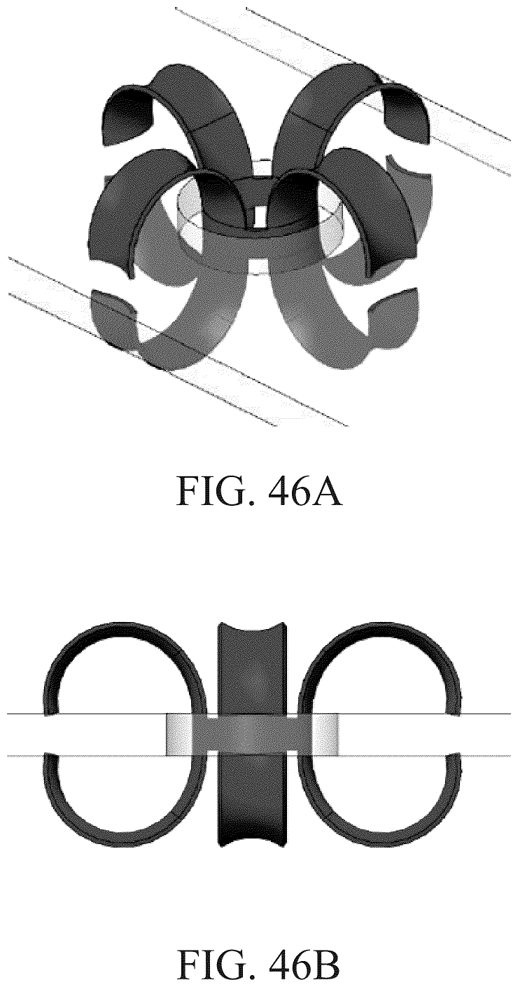

FIGS. 44A-44B depict a ratcheting otomy securement apparatus. An exemplary embodiment comprises a distal ratcheting member and a proximal ratcheting member connected by a central ratchet. The ratcheting securement apparatus is stored in a delivery device in a delivery configuration. The medical professional advances the delivery device through a created otomy. Once on the distal side of the otomy, the medical professional pulls back on the delivery device to deploy the distal ratcheting member on the distal side of the tissue surrounding the otomy into a deployed configuration. The medical professional may pull further back on the delivery device through to the proximal side of the otomy to deploy the proximal ratcheting member on the proximal side of the tissue surrounding the otomy into a deployed configuration. Once in the deployed configuration, the medical professional may ratchet the two ratcheting members toward each other. The central ratchet only allows for the ratcheting members to move into proximity, not further apart. Once in proximity to one another, the ratcheting members clamp the tissue surrounding the otomy, securing the otomy and reducing tissue damage.

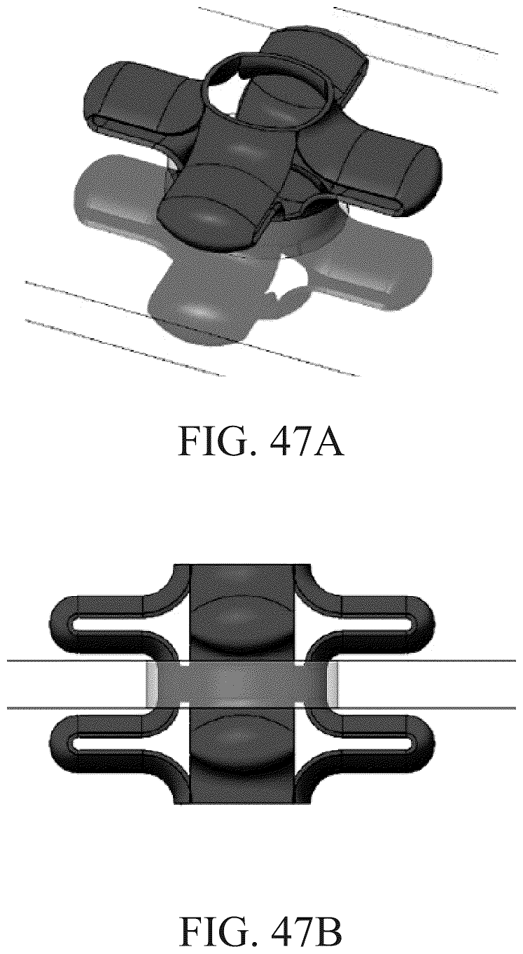

FIGS. 45A-45B depict a circular otomy securement device. The circular securement device may be made from nitinol, stainless steel, or other biocompatible materials. As is shown in FIG. 45A, the securement device may comprise two circular hoops parallel to each other and connected by one or more central supports. The circular securement device is stored in a delivery device in a delivery configuration. The medical professional advances the delivery device through a created otomy. Once on the distal side of the otomy, the medical professional pulls back on the delivery device to deploy the distal hoop into the distal side of the tissue surrounding the otomy into a deployed configuration. The medical professional may pull further back on the delivery device through to the proximal side of the otomy to deploy the proximal hoop in the proximal side of the tissue surrounding the otomy into a deployed configuration. Once in the deployed configuration, the hoops, connected by a central support, clasp the otomy. This secures the size and shape of the otomy and reduces tissue tearing. The central opening of the hoops allows for immediate passage of fluid and other materials through the otomy before permanent anastomosis formation.

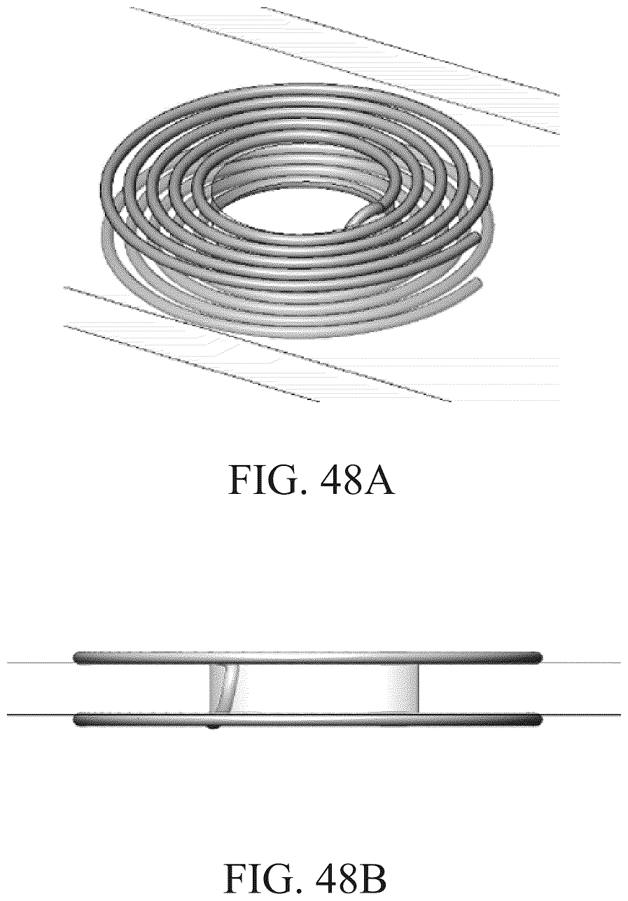

FIGS. 46A-46B depict a stent-style nitinol array. In an embodiment of the device, split nitinol tubing creates arms that may clamp tissue surrounding an otomy site. The arms are positioned around the circumference of a hollow central portion of the device. The stent-style nitinol array is stored in a delivery device in a delivery configuration. The medical professional advances the delivery device through a created otomy. Once on the distal side of the otomy, the medical professional pulls back on the delivery device to deploy distal arms of the device into the distal side of the tissue surrounding the otomy into a deployed configuration. The medical professional may pull further back on the delivery device through to the proximal side of the otomy to deploy proximal arms of the device in the proximal side of the tissue surrounding the otomy into a deployed configuration. Once in the deployed configuration, the arms clasp the tissue surrounding the otomy. This secures the size and shape of the otomy and reduces tissue tearing. The central section of the device allows for the passage of fluid or surgical tools through the otomy.

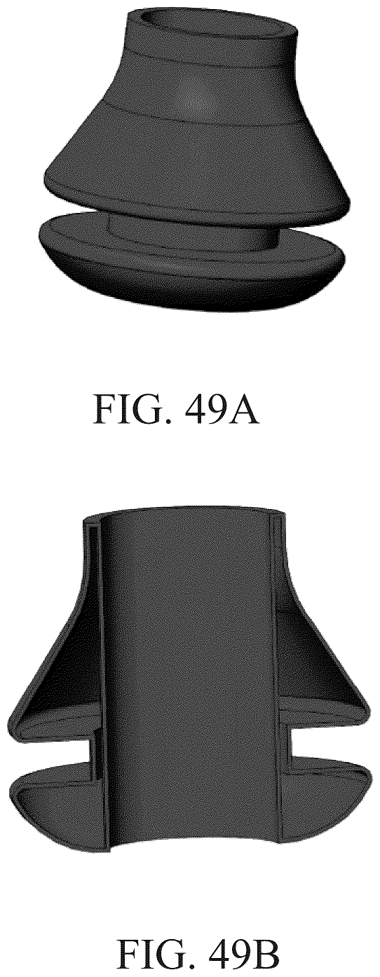

FIGS. 47A-47B depict an alternative embodiment of a stent-style nitinol array securement device. The embodiment may comprise nitinol tubing that deforms upon deployment to clamp the tissue surrounding an otomy. The apparatus may comprise two or more nitinol tubes around the circumference of a hollow center region. The stent-style nitinol array is stored in a delivery device in a delivery configuration. The medical professional advances the delivery device through a created otomy. Once on the distal side of the otomy, the medical professional pulls back on the delivery device to deploy the distal nitinol tubes into the distal side of the tissue where they deform into a deployed configuration. The medical professional may pull further back on the delivery device through to the proximal side of the otomy to deploy the proximal nitinol tubes into the proximal side of the tissue where they deform into a deployed configuration. Once in the deployed configuration, the tubes clasp the tissue surrounding the otomy. This secures the size and shape of the otomy and reduces tissue tearing. The center section allows for immediate passage of fluid and/or surgical tools through the otomy.

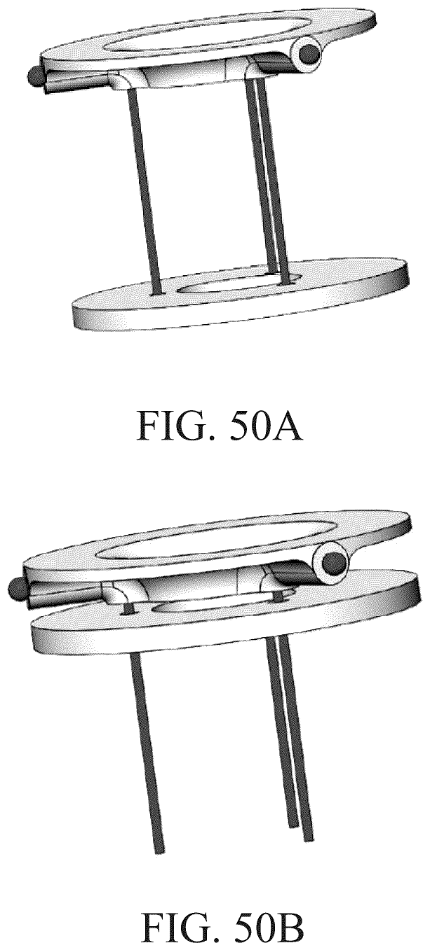

FIGS. 48A-48B depict a spiral style stent-style nitinol array securement device. A single length of nitinol, stainless steel, or other biocompatible material is coiled into two parallel, or substantially parallel, spiral arrays connected by a central connecting member. The stent-style nitinol array is stored in a delivery device in a delivery configuration. The medical professional advances the delivery device through a created otomy. Once on the distal side of the otomy, the medical professional pulls back on the delivery device to deploy the distal spiral wire array into the distal side into a deployed configuration. The medical professional may pull further back on the delivery device through to the proximal side of the otomy to deploy the proximal spiral style array into the proximal side of the tissue into a deployed configuration. Once in the deployed configuration, the arrays clasp the tissue surrounding the otomy.

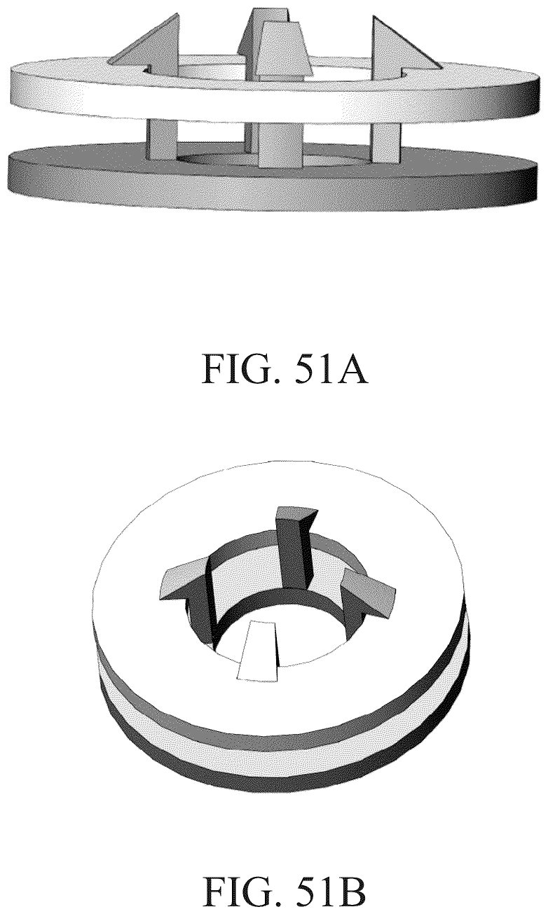

FIGS. 49A-49B depict an embodiment of an inflatable grommet for securing the tissue surrounding an otomy site. The grommet may comprise a distal flange and a proximal flange connected by a hollow central section. The inflatable grommet may be stored in a delivery device in a deflated, delivery configuration. The medical professional advances the delivery device through a created otomy. Once on the distal side of the otomy, the medical professional pulls back on the delivery device to deploy the distal flange of the inflatable grommet into the distal side of the otomy. The medical professional may pull further back on the delivery device through to the proximal side of the otomy to deploy the proximal flange of the inflatable into the proximal side of the tissue. The grommet may then be inflated into a deployed configuration. Once inflated, the flanges clamp the tissue surrounding the otomy, and hollow central portion allows for immediate passage of fluid and other materials through the otomy.

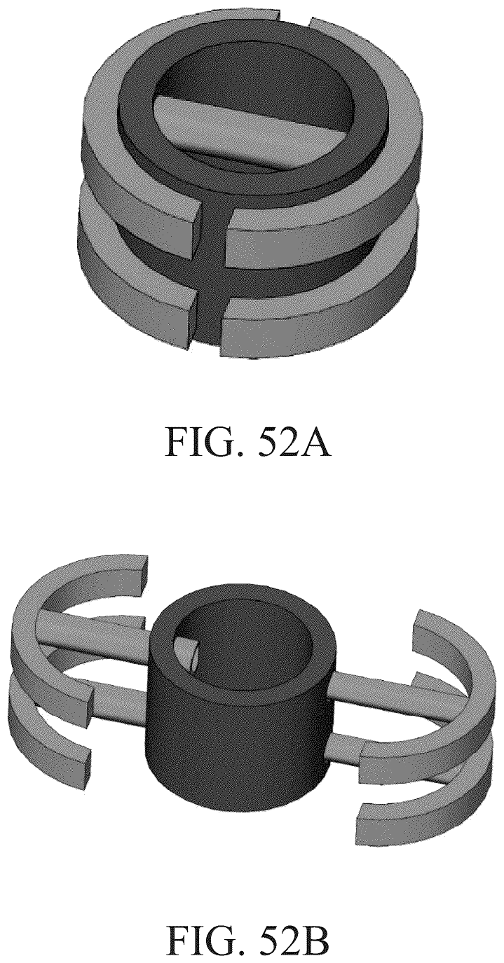

FIGS. 50A-50B depict an embodiment of a two-piece suture clamp. The clamp may comprise two washers made of stainless steel or another biocompatible material aligned parallel or substantially parallel to each other. The washers may be connected by one or more connecting members that are perpendicular to the washers, but parallel to other connecting members. The distal washer is attached to the connecting members. The washers may translate along the connecting members towards or away from the other washer. The medical professional positions a distal washer on the distal side of the created otomy with the connecting members positioned through the otomy. The medical professional then slides the proximal washer onto the connecting members, and translates the proximal washer towards the distal washer. The mated washers clamp tissue surrounding the otomy site, securing the tissue and preserving the otomy. The central opening of the washers allows for immediate passage of fluid and other material through the otomy.

In an alternative embodiment, the proximal washer is attached to the connecting members and the distal washer translates along the connecting members towards the proximal washer.

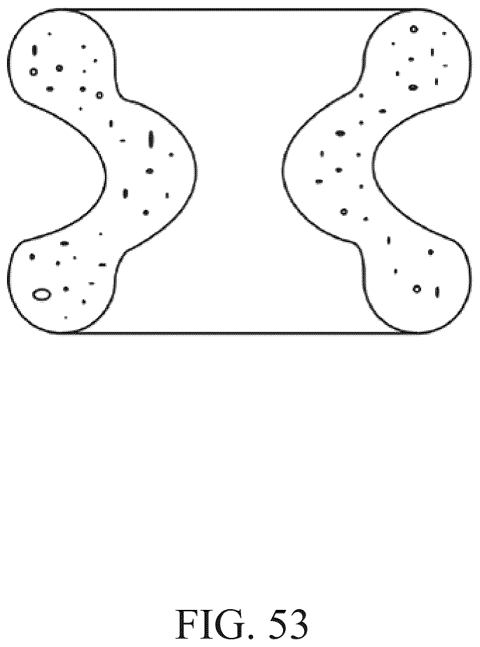

FIGS. 51A-51B depict a snap lock otomy clip. The otomy clip may comprise a distal washer and a proximal washer. The distal washer and/or the proximal washer may have snaps configured to mater with the inner circumference of the opposing washer. The medical professional positions the distal washer on the distal side of the otomy and the proximal washer on the proximal side of the otomy. The medical professional then brings the washers into proximity with each other, and slides the snaps into the inner circumference of the opposing washer, attaching the washers across the otomy. This secures the tissue surrounding the otomy site and reduces trauma to the tissue while preserving the otomy.

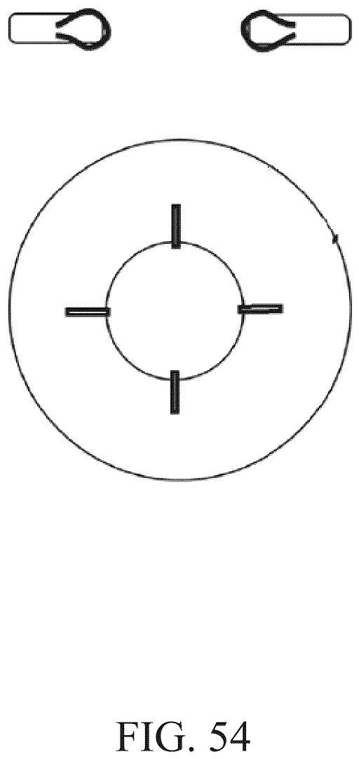

FIGS. 52A-52B depict an embodiment of a sliding arm otomy clip. The otomy clip comprises a hollow central support tube and four or more sliding arms, with two or more being proximal arms and two or more being distal arms. As shown in FIG. 52A, the sliding arms are in a delivery configuration for storage in a delivery device. The arms are curved to compliment the outer circumference of the hollow central support tube. The medical professional advances the delivery device into the otomy so that the distal arms are positioned on the distal side of the otomy and the proximal arms are positioned on the proximal side of the otomy. The medical professional may then deploy the arms of the sliding arm otomy clip into the deployed configurations, as shown in FIG. 52B. The sliding arms protrude from the hollow central support tube, and clamp the tissue surrounding the otomy. Note that the arms may deploy in any order.

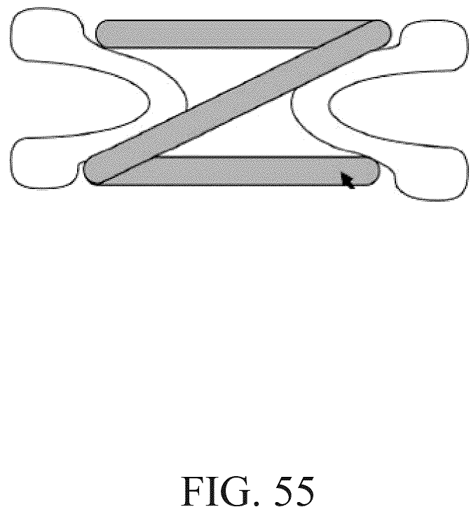

FIG. 53 depicts an embodiment of a foam grommet for securing tissue surrounding an otomy. The foam grommet is stored in a delivery device in a compressed delivery configuration. The medical professional advances the delivery device through the otomy to the distal side of the tissue. The medical professional then pulls back on the delivery device, deploying the distal end of the grommet into the distal lumen. The distal end of the grommet expands into a deployed configuration. The medical professional then pulls back on the delivery device, deploying the proximal end of the grommet into the proximal lumen. The proximal end of the grommet expands into a deployed configuration, as pictured in FIG. 53. The expanded foam grommet comprises a distal flange and a proximal flange, that together clamp and secure the tissue surrounding an otomy.

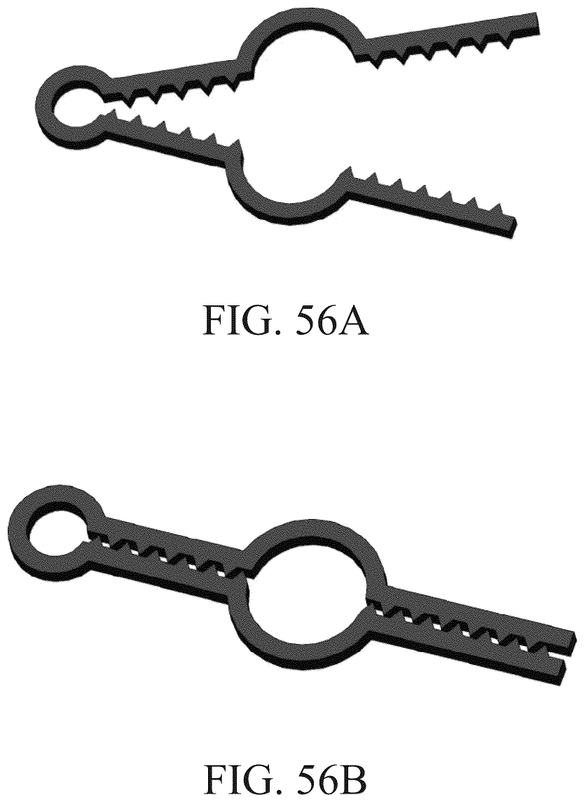

FIG. 54 depicts an embodiment of a clip secured grommet. The clip secured grommet may comprise one or more clips on the inner circumference of a hollow grommet. A one-sided grommet is sutured around the otomy, and the tissue surrounding the otomy is secured to the grommet with clips. The clips secure the tissue surrounding the otomy, reducing trauma and locomotion of the tissue to preserve the otomy.

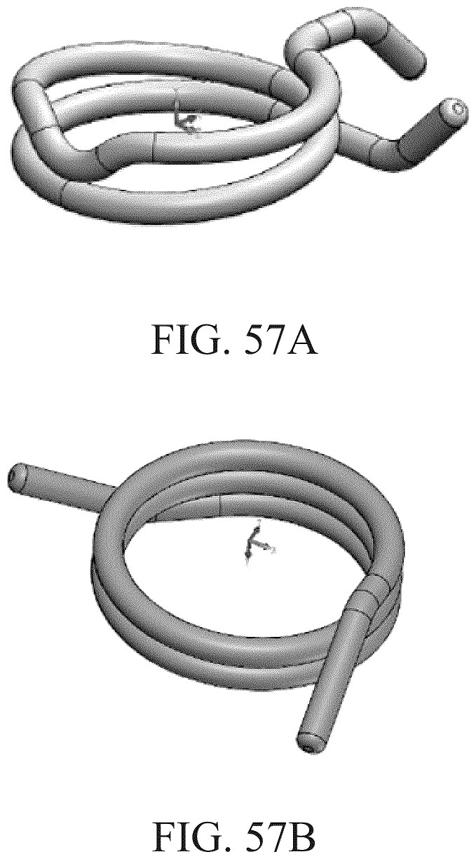

FIG. 55 depicts an embodiment of a coil compressed grommet. A grommet is stored in a delivery device in a compressed delivery configuration. The medical professional advances the delivery device through the otomy to the distal side of the tissue. The medical professional then pulls back on the delivery device, deploying the distal end of the grommet into the distal lumen. The distal end of the grommet expands into a deployed configuration. The medical professional then pulls back on the delivery device, deploying the proximal end of the grommet into the proximal lumen. The proximal end of the grommet expands into a deployed configuration. The grommet is then secured with a coil to preserve the otomy and secure the tissue surrounding the otomy.

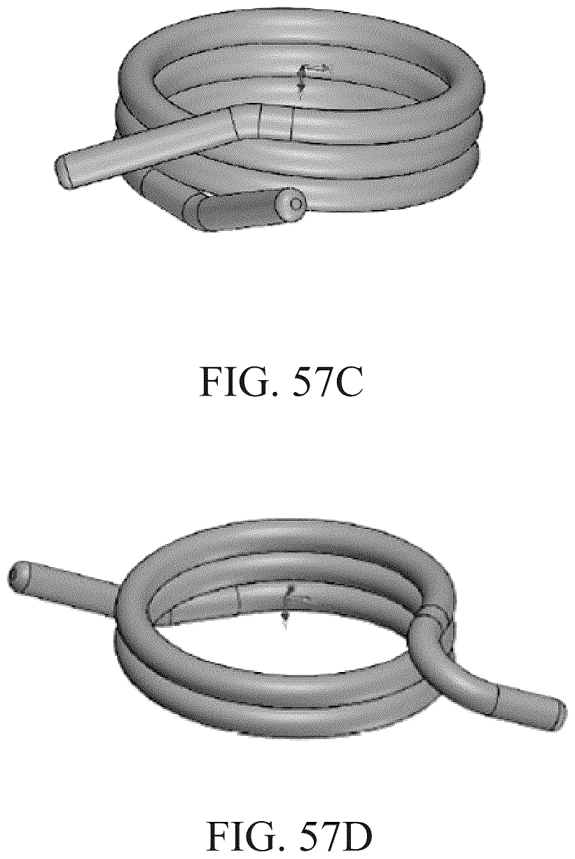

FIGS. 56A-56B depict an embodiment of a sprung clamping otomy clip. An otomy clip may be deployed around an otomy site to secure tissue. The otomy clip comprises a fenestrated design to allow for fluidic passage through the otomy. The otomy clip clamps the tissue around the otomy site, reducing tearing, dilation, or locomotion of the otomy site.

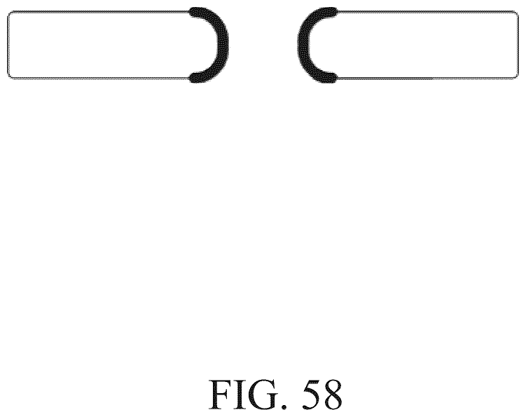

FIGS. 57A-57D depict illustrative embodiments of coiled wire otomy securement devices. A single length of nitinol, stainless steel, or other biocompatible material may be coiled and deployed around the otomy site to secure tissue. Layers of the coiled clamp tissue therebetween to secure the tissue and reduce trauma or locomotion of the tissue to secure the otomy. The hollow center of the coils allows for immediate passage of fluids and other materials through the otomy before creation of a permanent anastomosis.

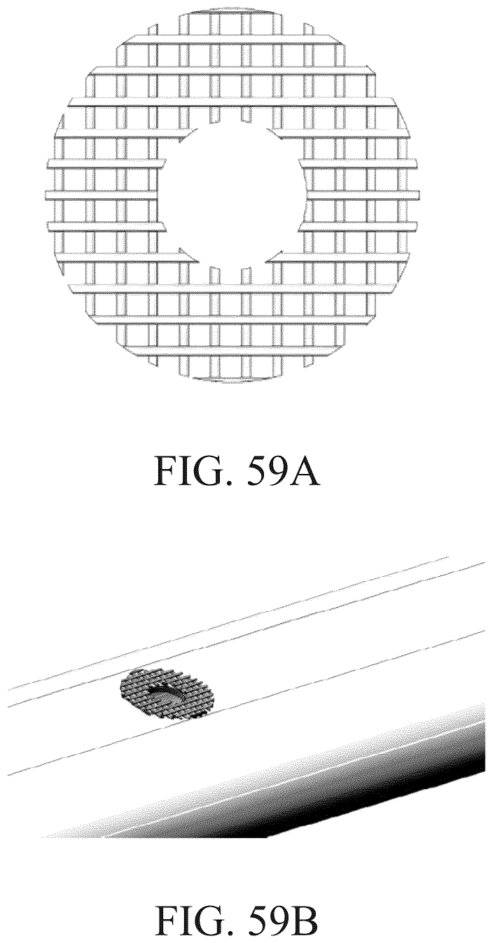

FIG. 58 depicts an embodiment of an adhesive reinforced otomy. After creation of an otomy, the tissue surrounding the otomy is coated with an adhesive to reduce tears and/or stretching of the otomy site. Adhesive on the tissue leaves the center of the otomy site open, allowing for passage of fluid and other materials through the otomy, while preserving the integrity of the otomy site.

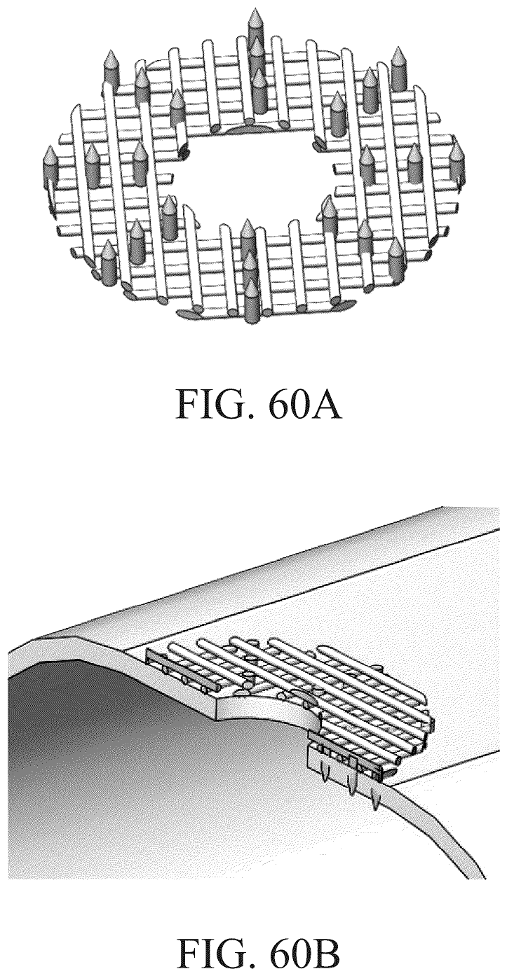

FIGS. 59A-59B depict an embodiment of a patch for securing an otomy. After creation of an otomy, the otomy site is supported by a patch adhered to the tissue surrounding the otomy site. The patch has an annular shape, allowing for passage of fluid and other materials through the patch and the otomy.

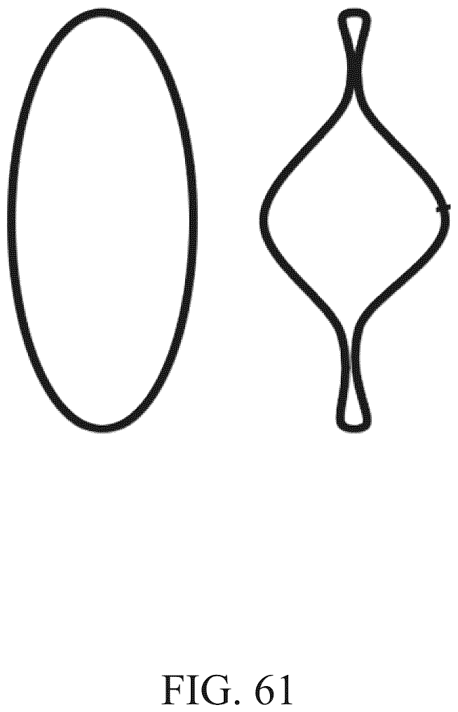

FIGS. 60A-60B depict an embodiment of a puncture patch for securing an otomy. After creation of an otomy, the otomy site is supported by a patch mechanically adhered to the tissue surrounding the otomy site. The patch may comprise spikes or other protrusions capable of puncturing the tissue surrounding the otomy site to secure the patch to the tissue, preserving the otomy.

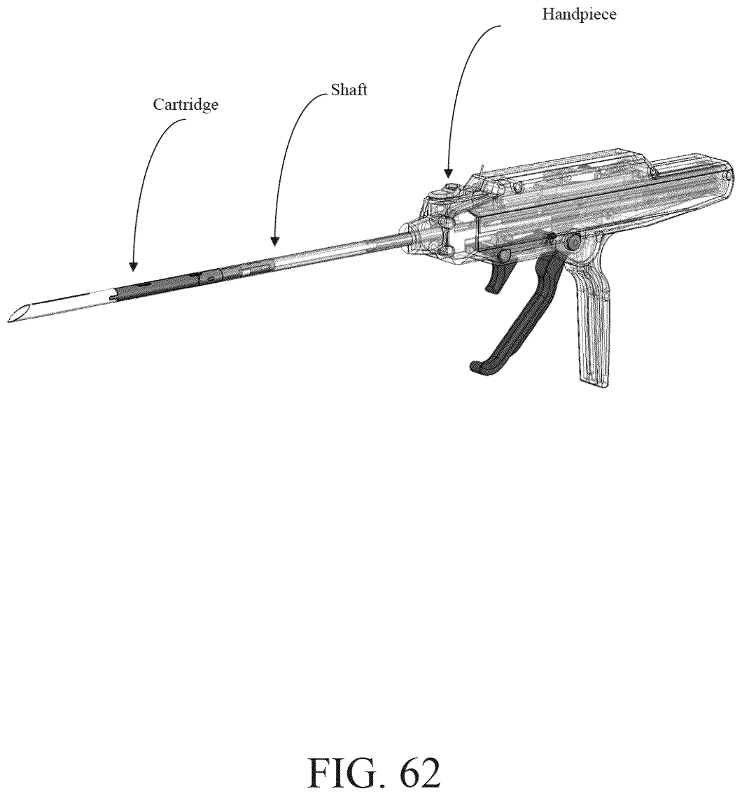

FIG. 61 depicts an embodiment of a crimped wire form otomy clip. The crimped wire otomy clip is stored in an annular delivery configuration in a delivery device. The crimped wire otomy clip may be deployed prior to or subsequent to the creation of the otomy. On delivery, the crimped wire otomy is clipped into a deployed configuration. The crimps clamp tissue surrounding the otomy site, securing the tissue and supporting the otomy. The ring shape of the clip allows for immediate passage of fluid or other materials through the clip and the otomy.

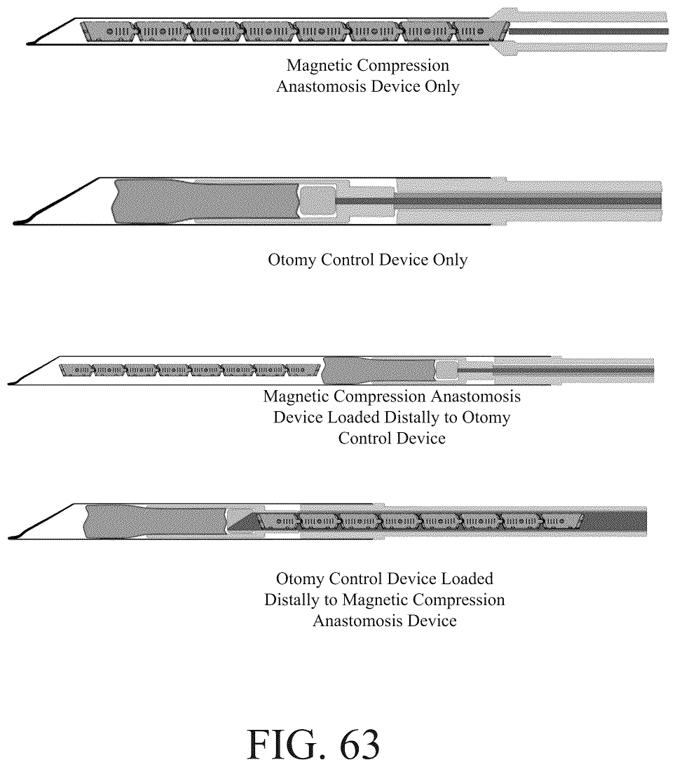

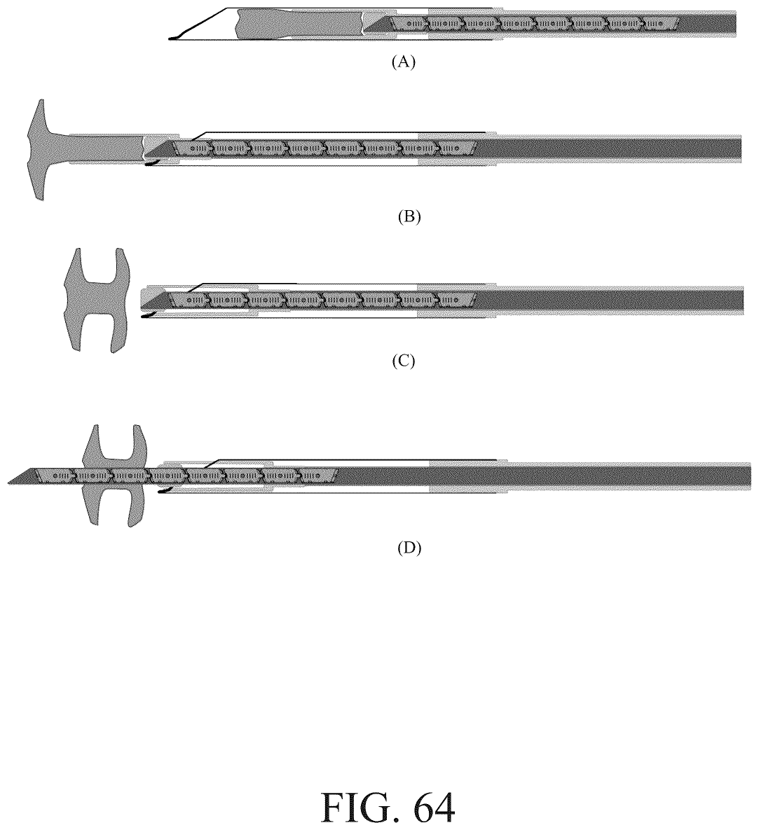

Certain embodiments provide an apparatus and system for delivery of both magnetic compression anastomosis devices (e.g., of the types described with reference to FIGS. 1-21 which generally can be delivered in a linear arrangement but convert such as by self-assembly into a geometric configuration such as a polygon or circle upon deployment, e.g., a Flexagon™ magnetic compression anastomosis device) and otomy control devices (e.g., of the types described with reference to FIGS. 22-61, e.g., an OTOLoc™ otomy control device). As shown in FIG. 62, certain embodiments are configured to utilize different types of anastomosis procedure cartridges with a universal delivery device that can operate all of the different types of cartridges. The universal delivery device includes a handpiece having a shaft that is configured to attach the various anastomosis procedure cartridges and through which the cartridge is operated via the handpiece. As depicted schematically in FIG. 63, in certain embodiments, the handpiece can be used with the following anastomosis procedure cartridge configurations:
a cartridge containing just a magnetic compression anastomosis device (e.g., a self-assembling magnetic compression anastomosis device such as a “Flexagon” self-assembling magnetic compression anastomosis device);
a cartridge containing just an otomy control device (e.g., a grommet-type OTOLoc™ otomy control device as described above with reference to FIGS. 22-24);
a cartridge containing a magnetic compression anastomosis device distal to an otomy control device (e.g., a single cartridge that deploys a Flexagon™ device prior to the deployment of an OTOLoc™ device);
a cartridge containing an otomy control device distal to a magnetic compression anastomosis device without otomy alignment (e.g., a single cartridge that deploys an OTOLoc™ device and then requires the user to access the OTOLoc™ device prior to deploying a Flexagon™ device through the OTOLoc™ device);
a cartridge containing an otomy control device distal to a magnetic compression anastomosis device with thru deployment (e.g., a single cartridge that deploys the OTOLoc™ distal flange, then allows a Flexagon™ device to be deployed through the OTOLoc™ device, then allows deployment of the proximal flange and release of the OTOLoc™ device); and
a cartridge containing an otomy control device distal to a magnetic compression anastomosis device with alignment (e.g., a single cartridge that deploys an OTOLoc™ device and maintains a connection with the OTOLoc™ device for deployment of the Flexagon™ device through the OTOLoc™ device and subsequent release of the OTOLoc™ device).

Thus, certain embodiments allow for delivery of just a magnetic compression anastomosis device, delivery of just an otomy control device, delivery of an otomy control device followed by delivery of a magnetic compression anastomosis device (e.g., to the distal side of the otomy control device through the otomy control device itself), and delivery of a magnetic compression anastomosis device through the otomy followed by delivery of an otomy control device. Different versions of cartridges can include different types of magnetic compression anastomosis devices (e.g., cartridge configurations with single-element devices, cartridge configurations with multiple-element devices which can include both self-assembling and non-self-assembling devices, etc.), different types of otomy control devices (e.g., cartridge configurations with different otomy control device variants), etc. It should be noted that embodiments are not limited to delivery of a magnetic compression anastomosis device but instead embodiments could be configured for delivery of other types of devices before or after delivery of the otomy control device (e.g., a surgical device, a lumen support device, etc.). It also should be noted that cartridge configurations can include multiple of the same type of element, e.g., two otomy control devices (e.g., allowing delivery of two OTOLoc devices without changing cartridges), two magnet compression anastomosis devices (e.g., allowing delivery of two Flexagon devices without changing cartridges), two otomy control devices and two magnetic compression anastomosis devices (e.g., a distal OTOLoc, a distal Flexagon, a proximal OTOLoc, and a proximal Flexagon allowing delivery of an OTOLoc and a Flexagon into two otomies to be connected without changing cartridges), etc. The magnetic compression anastomosis device or other device delivered via this single apparatus may be, or may become, wider than the otomy control device opening after delivery of the magnetic compression anastomosis device or other device and therefore such device may be physically or automatically prevented from passing through the otomy control device due to its shape or size, e.g., prevented from exiting the lumen through the otomy control device.

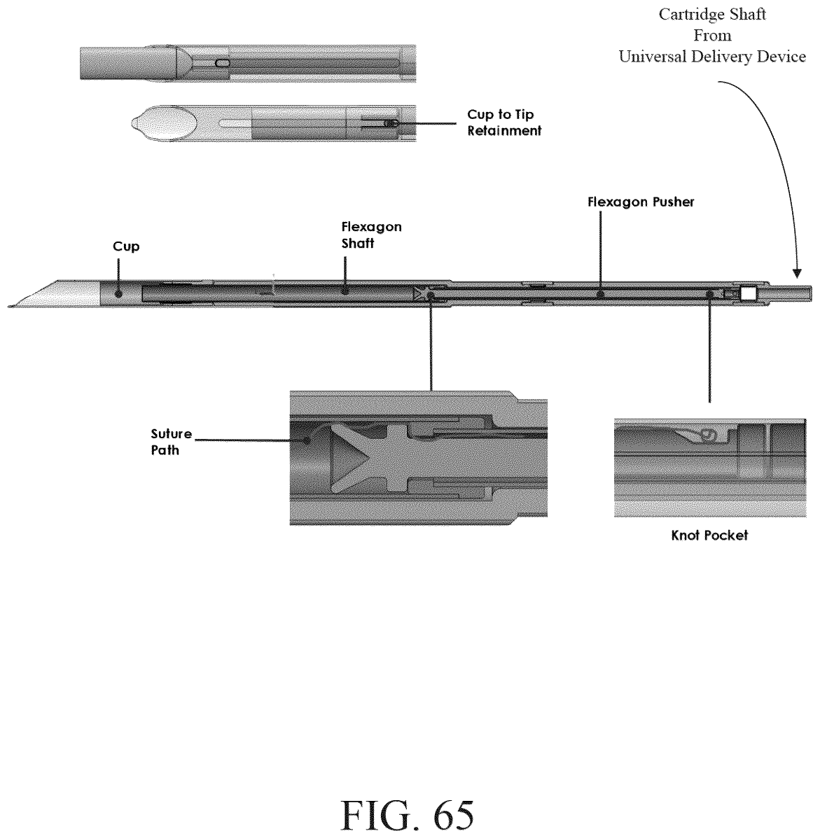

FIG. 64 is a schematic diagram showing operation of a cartridge containing an OTOLoc distal to a Flexagon without otomy alignment in accordance with certain embodiments. FIG. 64(A) shows the preloaded cartridge with the OTOLoc distal to the Flexagon and contained within a pusher that is advanced when the Flexagon is advanced from the handpiece. FIG. 64(B) shows the Flexagon and OTOLoc pusher being advanced such that the distal portion of the OTOLoc is deployed, e.g., on the side of the otomy within the lumen. At this point, the handpiece with cartridge may be retracted and then the Flexagon advanced further to deploy the proximal portion of the OTOLoc as shown in FIG. 64(C), e.g., on the side of the otomy exterior to the lumen so as to capture the tissue and control the otomy. The cartridge then may be aligned with the opening in the OTOLoc and optionally passed partially or fully through the opening and then the Flexagon advanced to fully deploy the Flexagon within the lumen through the OTOLoc as shown in FIG. 64(D).

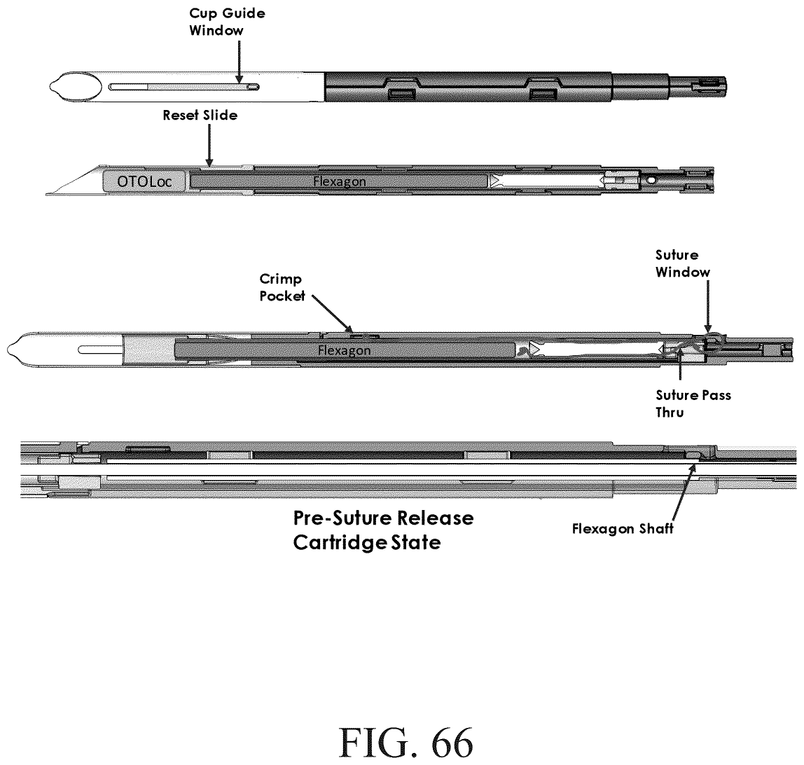

FIG. 65 is a schematic diagram showing one cartridge layout in accordance with certain embodiments. This diagram shows the basic layout of the lengths and control of motion being transferred from the handpiece. In this embodiment, there are two forward motions interfacing with the Flexagon shaft and pusher. Motion is reversed by retracting the cartridge back into the handpiece shaft. The cup “floats” in the tip. The device is deployed by the Flexagon shaft and is retracted by pulling back on the entire cartridge. Suture length is stored between the Flexagon shaft and pusher and is retained when locating the deployed Flexagon due to the knot wedging itself between the shaft and pusher. The suture is released when the knot pocket passes the pinch point in the Flexagon shaft.

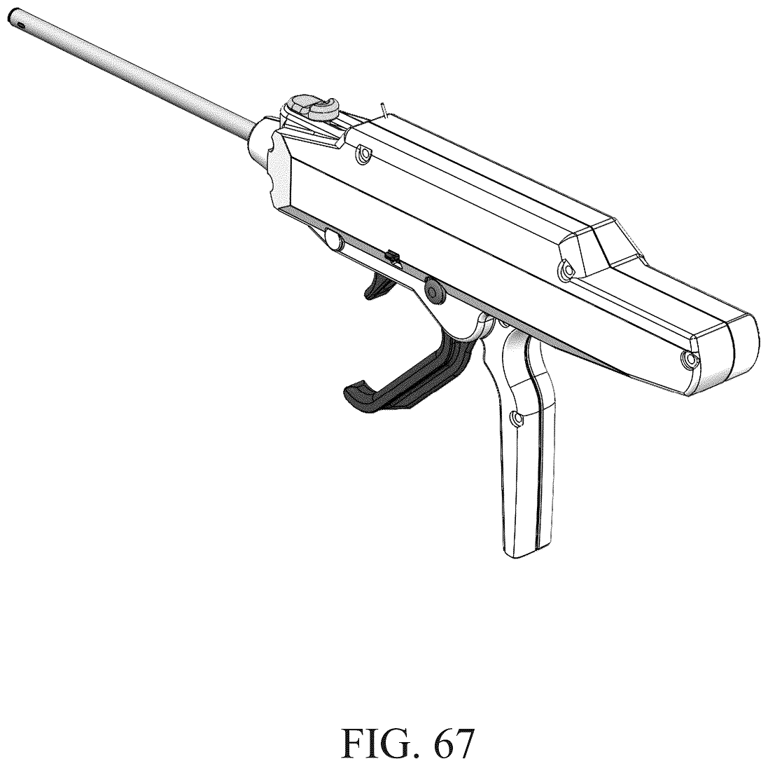

FIG. 66 is a schematic diagram showing one cartridge containing an OTOLoc distal to a Flexagon in accordance with certain embodiments. Here, the suture is routed through the Flexagon tube out the proximal cap around the suture window and back down the side of the Flexagon tube. A ball crimp at the end of the suture is captured in the crimp pocket and is not released until the Flexagon is fully deployed. The crimp can then travel through the suture window as the surgeon seats the Flexagon. When the suture is released, the crimp can bypass the Flexagon Shaft and be fully released.

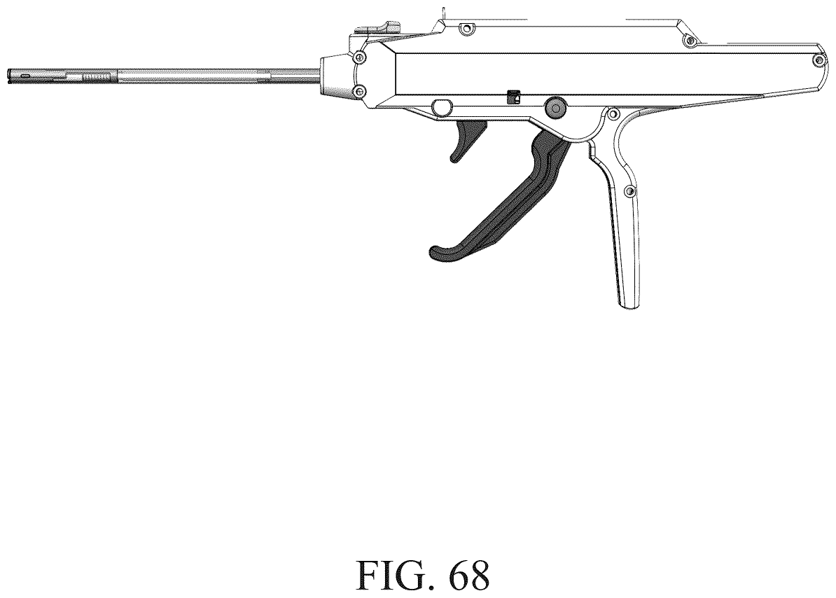

FIG. 67 is a perspective view of the universal delivery device of FIG. 62.

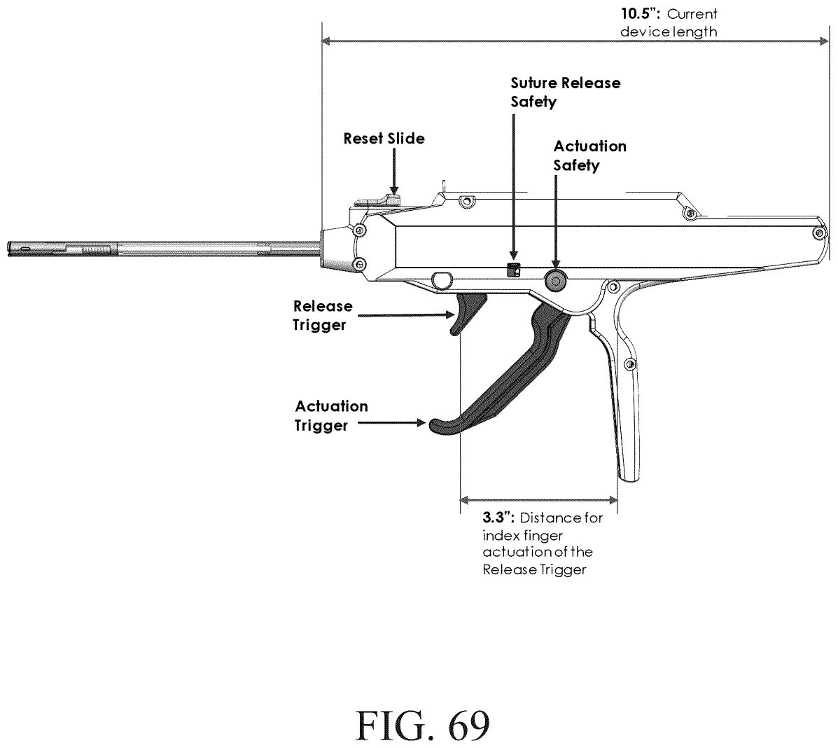

FIG. 68 is a side-view of the universal delivery device of FIG. 62.

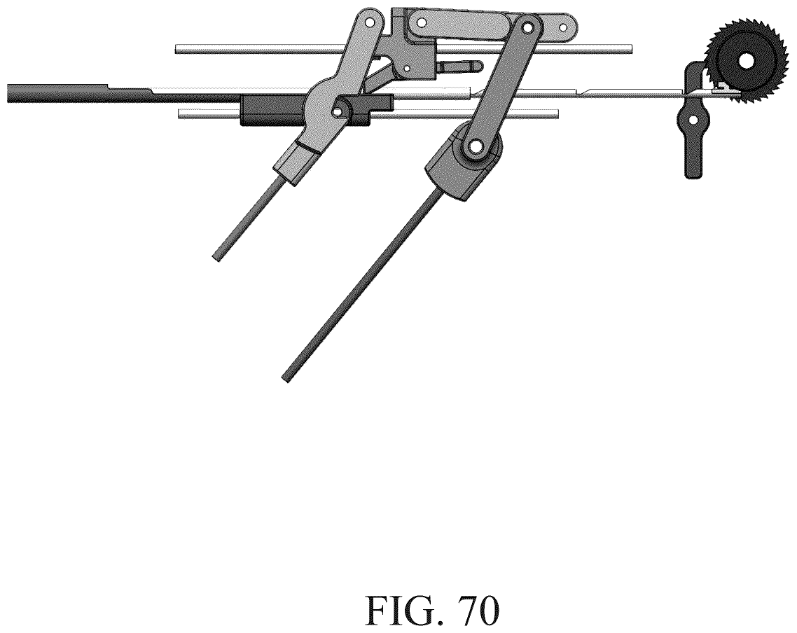

FIG. 69 is an annotated version of the side-view of FIG. 68 highlighting certain components and dimensions of this particular embodiment, particularly identifying an actuation trigger (which acts as a forward motion trigger), a release trigger (which acts as a reverse motion trigger), an actuation safety to prevent premature actuation operation, a suture release safety to prevent premature release of a magnetic compression anastomosis device suture, and a reset slide for resetting the delivery mechanism such as to remove a used cartridge and install a new cartridge. Generally speaking, this universal delivery device is designed to look and feel like a laparoscopic stapler, e.g., so that surgeons will feel comfortable and confident using the device. It also is designed for one-handed operation and has an ambidextrous design. It also includes multiple safety locks to ensure proper deployment of devices. An exemplary procedure might involve accessing the otomy, disengaging the actuation safety, squeezing the actuation trigger to advance the OTOLoc, squeezing the release trigger to deploy the OTOLoc, squeezing the actuation trigger one or more (e.g., three) times to deploy the Flexagon, disengaging the suture release safety, removing the device from the otomy, and actuating the reset slide to unlock/remove the cartridge and reload another cartridge. The internal mechanisms of the universal delivery device are configured to support these operations, e.g., using a ratcheting feature or other mechanism to control advancement of the OTOLoc and Flexagon devices in the cartridge.

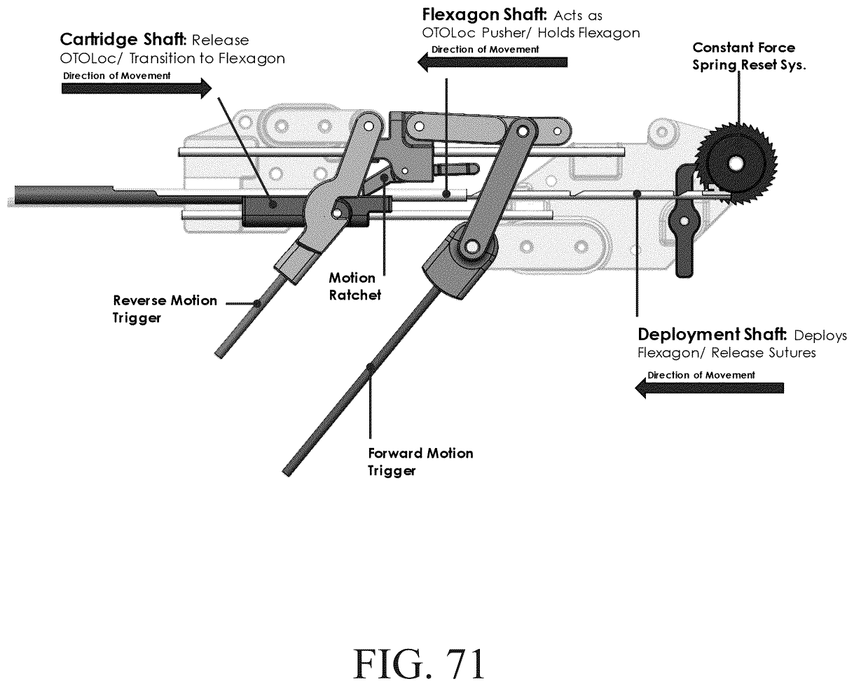

FIGS. 70-71 are schematic diagrams showing a linkage drive mechanism for controlling actuation and release operations.

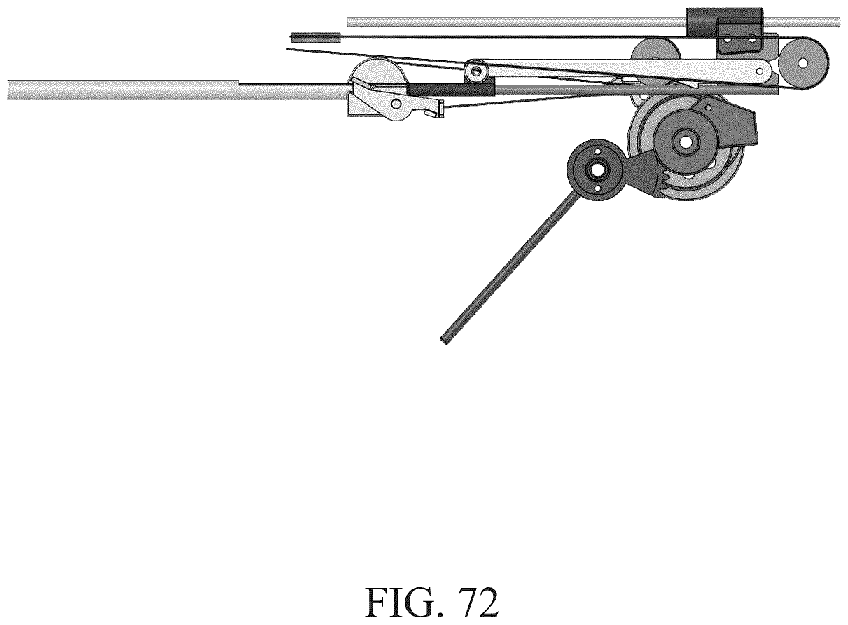

FIGS. 72-73 are schematic diagrams showing a cable drive mechanism for controlling actuation and release operations.

In any of the drive mechanisms (e.g., the linkage drive mechanism shown in FIGS. 70-71 and the cable drive mechanism shown in FIGS. 72-73), one or more mechanical elements that transmit manual inputs can be replaced with one or more motor-driven elements that convert other forms of energy into mechanical energy to produce the corresponding motion such as an electric motor or a motor that converts other forms of stored energy (e.g., spring, hydraulic, pneumatic, etc.) into mechanical energy. Thus, for example, the pusher can be advanced through manually-driven motion, by electric motor (e.g., controlled by a trigger of the device), by a spring-loaded mechanism (e.g., released by a trigger of the device), etc.

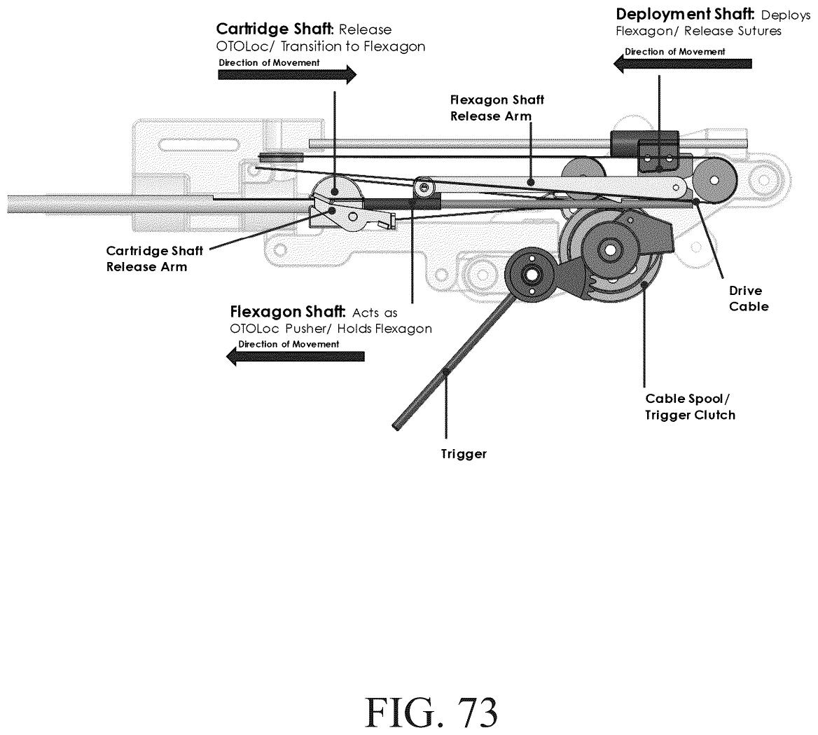

FIG. 74 is a schematic diagram showing deployment of an OTOLoc followed by deployment of a Flexagon similar to that of FIG. 64 but with reference to the components of the universal delivery device shown in FIG. 69, i.e., depicting one manner in which the universal delivery device of FIG. 69 can be used to deploy an OTOLoc and/or Flexagon.

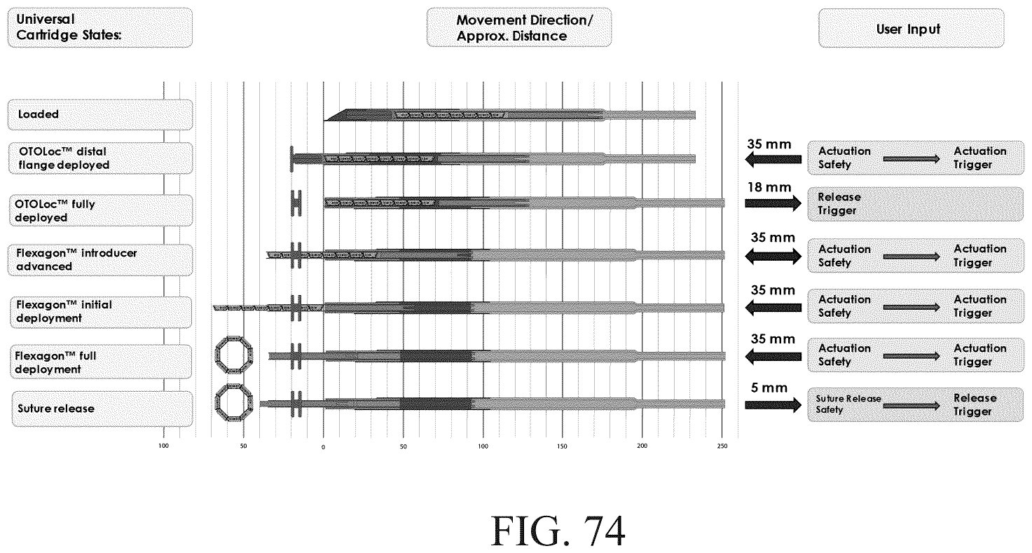

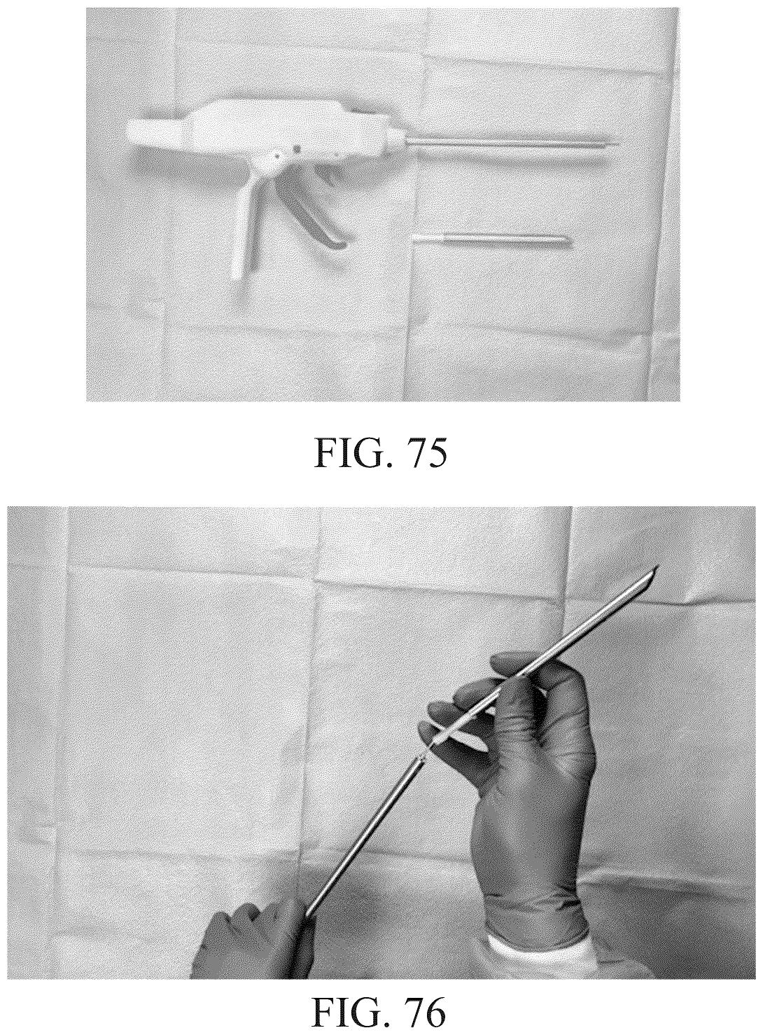

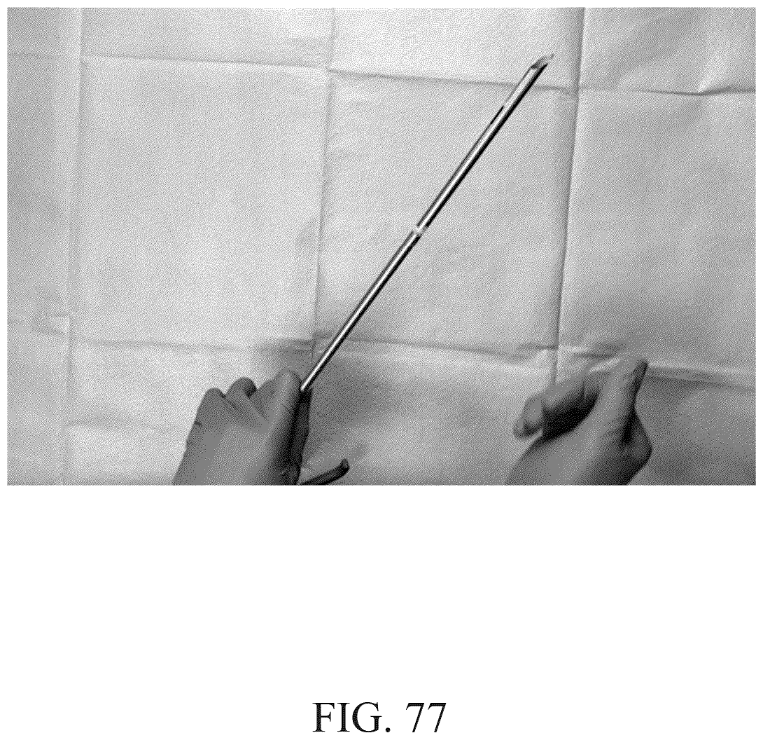

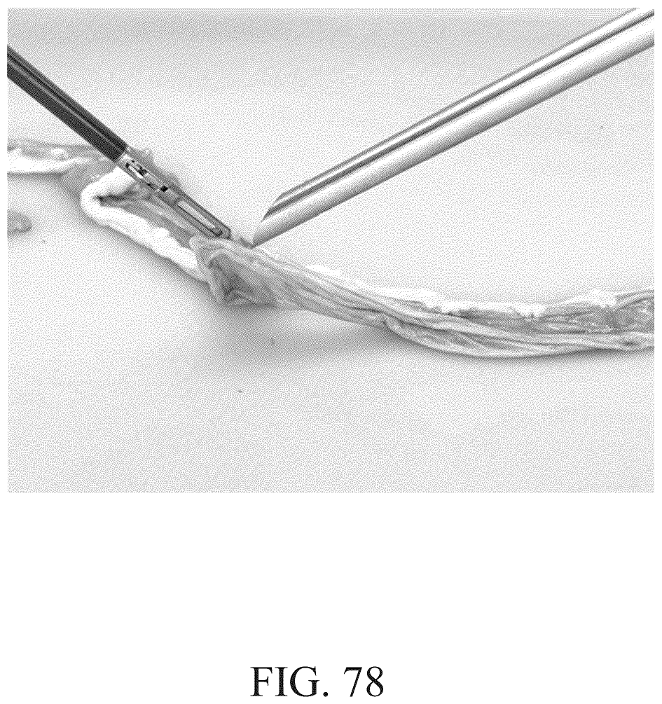

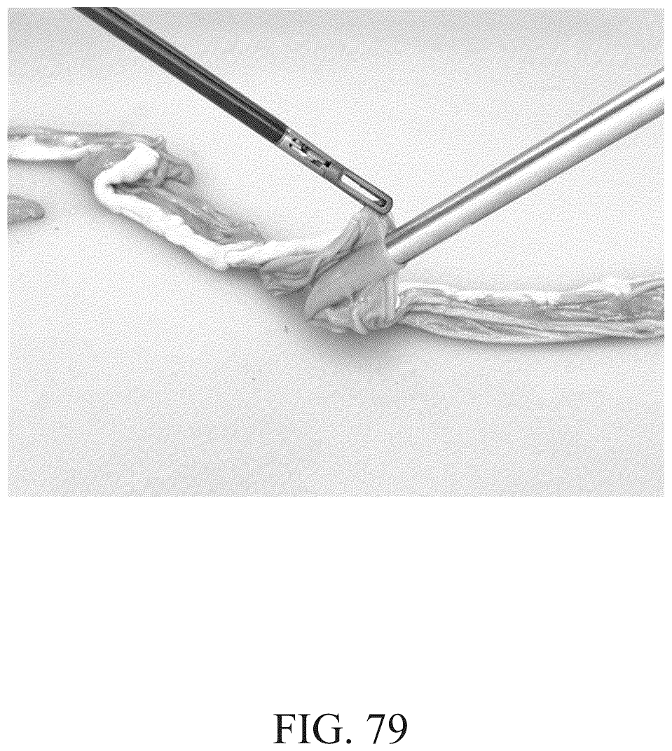

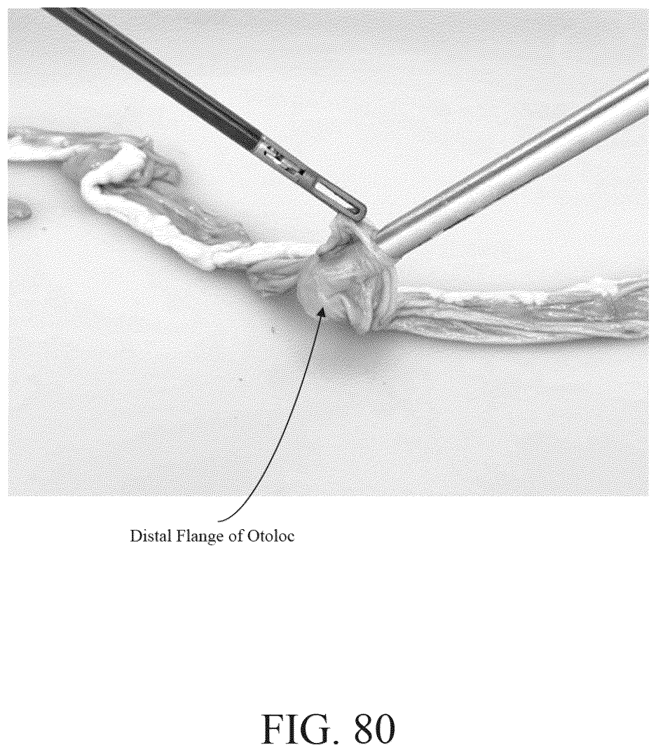

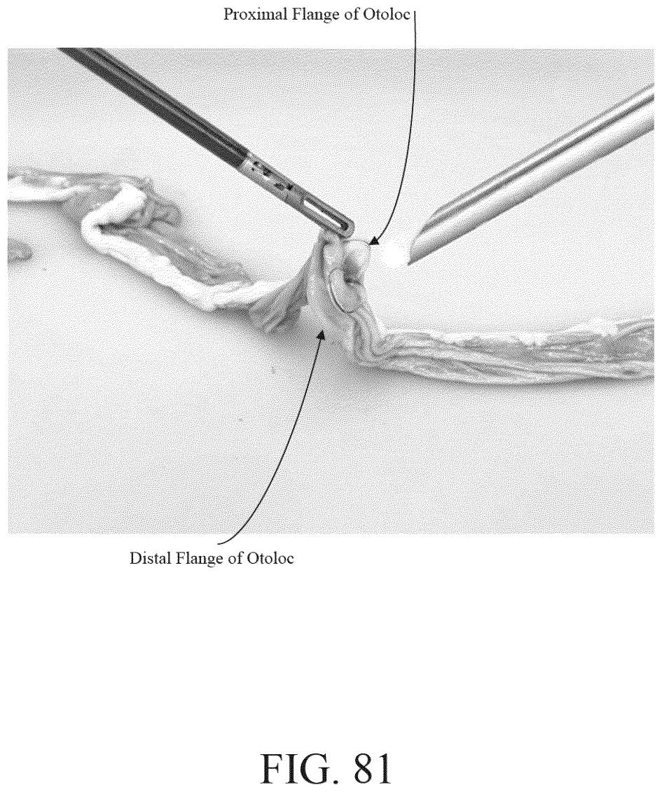

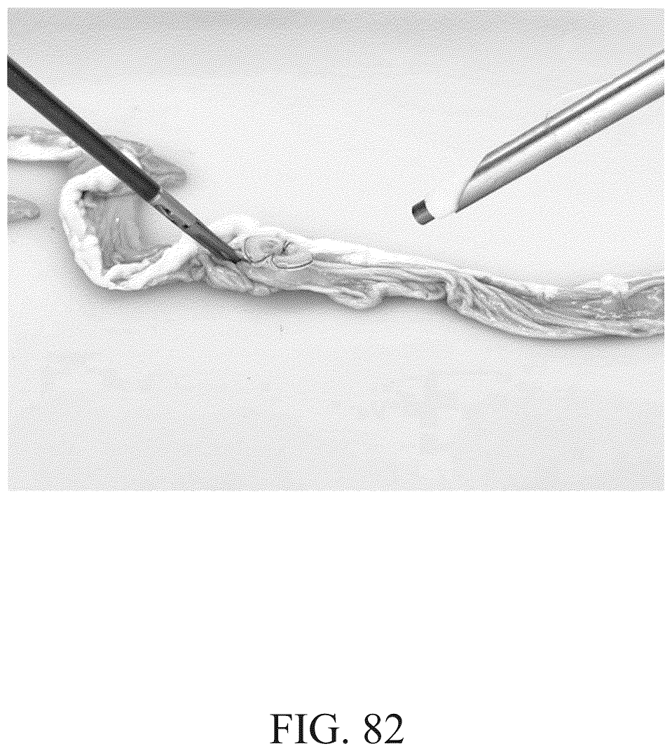

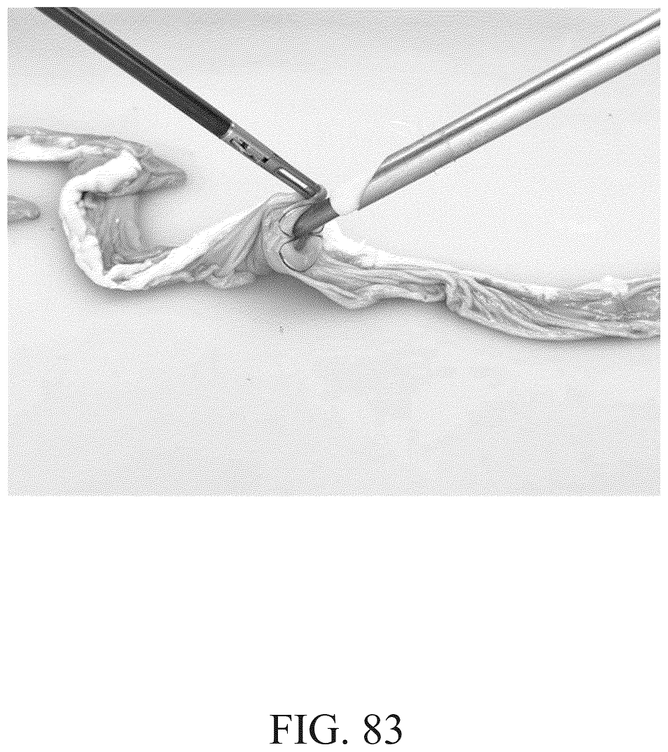

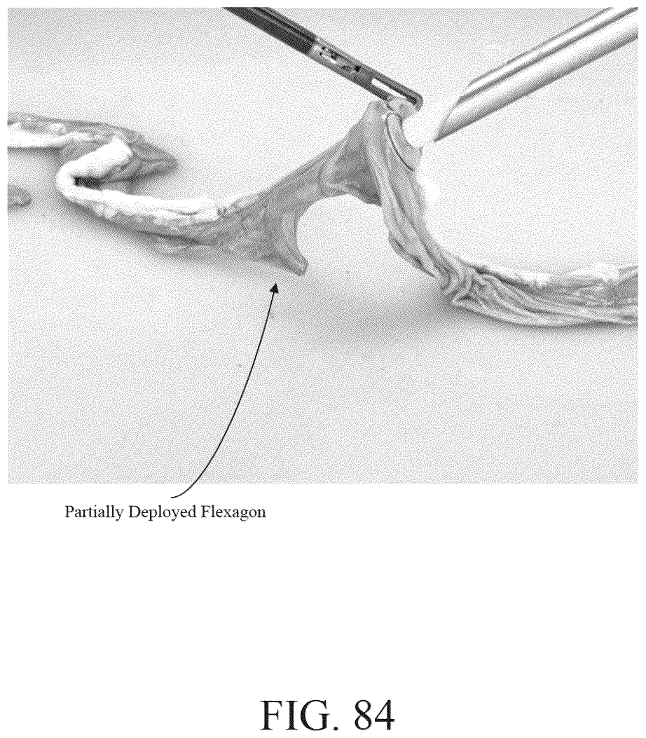

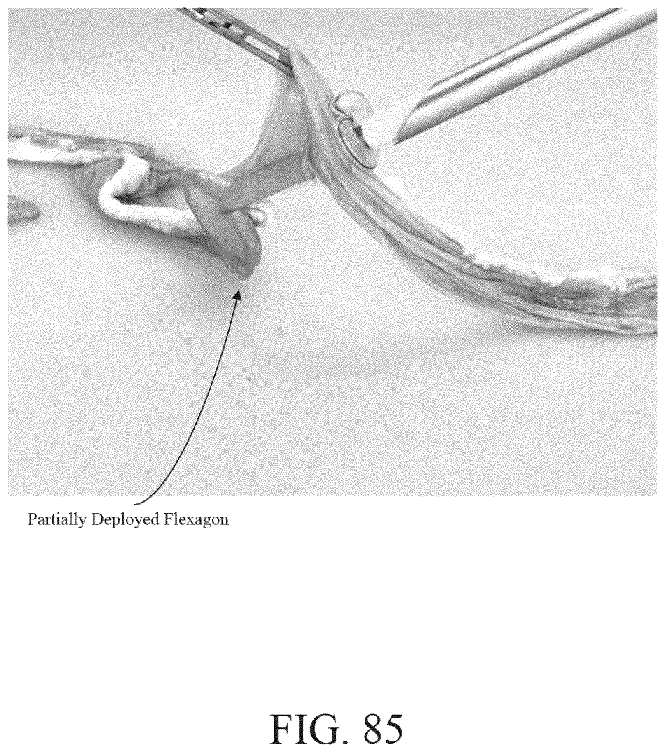

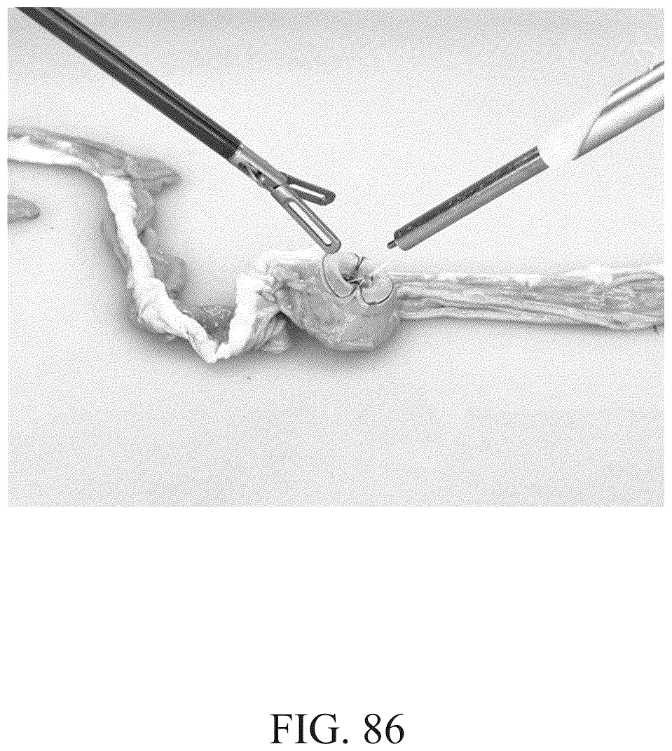

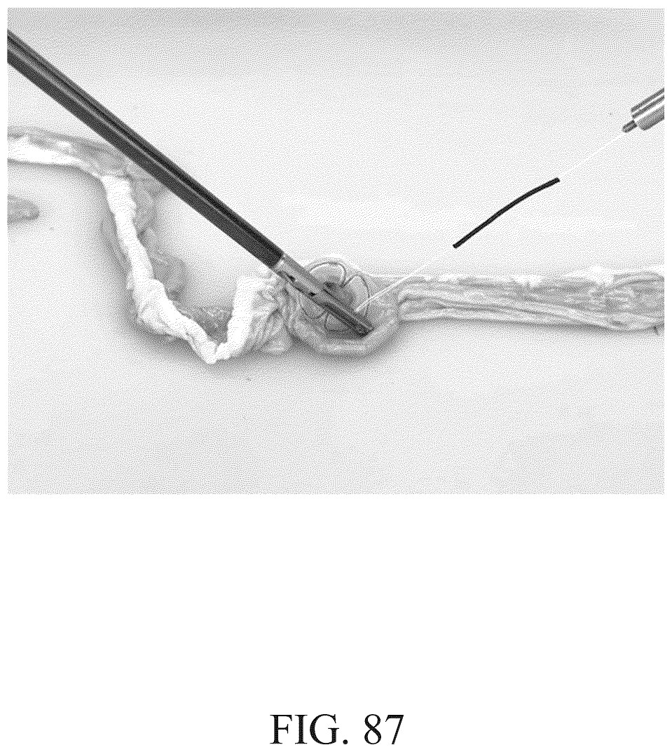

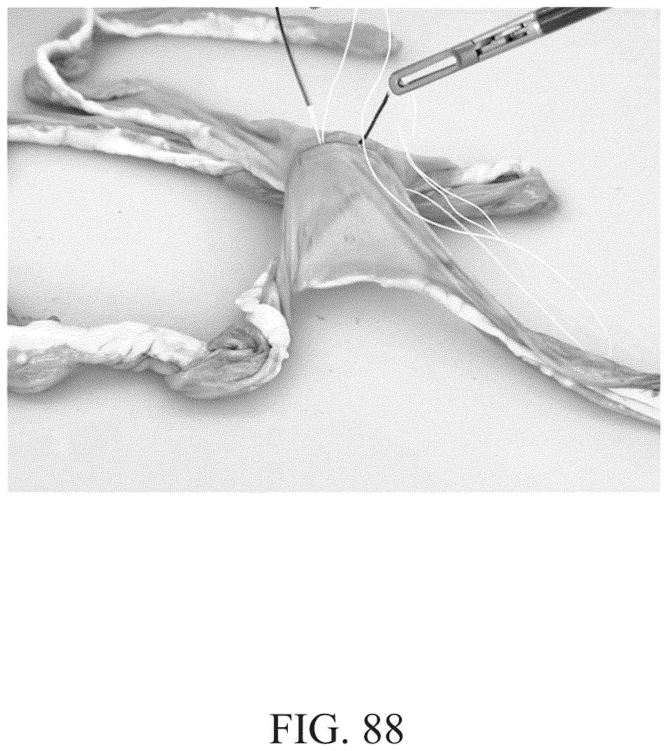

The following is a detailed description for general usage of the universal delivery device and cartridge in accordance with certain embodiments. Pre-operation, the user would remove the handpiece from packaging and would remove a cartridge from packaging. FIG. 75 shows a universal delivery device and a cartridge pre-operation, in accordance with certain embodiments. The user would then load the cartridge onto the handpiece, e.g., aligning the cartridge with the shaft, inserting the cartridge onto the shaft until bottomed, rotate the cartridge clockwise until stop, and slide the shipping cover distally off of the cartridge. FIG. 76 shows the cartridge of FIG. 75 being installed onto the universal delivery device. FIG. 77 shows the cartridge of FIG. 76 fully installed on the universal delivery device. During the operation, the user would select target anatomy, e.g., using graspers to ensure first and second enterotomy sites can be connected. The user then would create the enterotomy, e.g., using hook cautery to burn through one wall of the target anatomy and then dilating the enterotomy to the cautery tool shaft diameter by passing the tool through the enterotomy into the interior of the target anatomy. The user then would engage the enterotomy, e.g., by swapping the hook cautery tool for the OTOLoc delivery tool and presenting the enterotomy towards the OTOLoc cartridge tip with the grasper located approximately 2 cm away from the enterotomy (FIG. 78) and then inserting the shoehorn tip of the cartridge into the enterotomy while apply light tension from the graspers, e.g., along at the 12 o'clock position (FIG. 79). The user then would continue inserting the OTOLoc cartridge tip into the enterotomy, e.g., while maintaining light tension, optionally rotating/rocking the handpiece in both directions while inserting which should facilitate the passage of the tool through the enterotomy. The user then will position the cartridge for deployment of the OTOLoc, e.g., using the graspers to pull the tissue adjacent to the enterotomy proximally along the tip of the OTOLoc cartridge to a designed band on the tip. The user then would deploy the distal flange of the OTOLoc device (FIG. 80), e.g., by depressing the actuation safety lock and pulling the actuation trigger arm fully and then releasing it. The user then would seat the OTOLoc, e.g., while applying light tension with the graspers, retract/slide the OTOLoc cartridge out of the enterotomy until the distal flange of the OTOLoc tents the tissue around the enterotomy and the tip of the OTOLoc cartridge is fully visible, then using the graspers to smooth out/remove any gathers or folds in the tissue around the enterotomy on the distal flange. The user then would deploy the proximal flange (FIG. 81) to fully release the OTOLoc (FIG. 82), e.g., by pulling the release trigger to a hard stop. The user then would extend the Flexagon shaft, e.g., by depressing the actuation safety lock and pulling the actuation trigger arm fully and then releasing. The user then would access the OTOLoc, e.g., while holding the anatomy approximately 2 cm away from the OTOLoc, and insert the Flexagon shaft through the OTOLoc to a designated marking on the Flexagon shaft (FIG. 83). The user then would check the Flexagon deployment pocket, e.g., to ensure there is space below the Flexagon shaft within the anatomy into which the Flexagon can deploy. The user then would partially deploy the Flexagon (FIG. 84), e.g., by depressing the actuation safety lock and pulling the actuation trigger arm fully and then releasing. The user then would inspect the anatomy at the deployment site, e.g., check the area around the tip of the Flexagon shaft to ensure the anatomy has not been pinched and is not restricting the deployment of the Flexagon. The user then would fully deploy the Flexagon (FIG. 85), e.g., by depressing the actuation safety lock and pulling the actuation trigger arm fully followed by release. The user then would seat the Flexagon, e.g., retract the Flexagon shaft out of the OTOLoc and apply tension to the suture lines to seat the Flexagon against the distal flange of the OTOLoc (FIG. 86). The user then would transfer the suture lines, e.g., transfer control of the suture lines to a grasper, and then would release the suture lines, e.g., by pulling the release trigger and removing the tool from the surgical site (FIG. 87). Now, post operation, the user would reset the tool, e.g., by sliding and holding the reset button proximally to the hard stop and pulling on the tool shaft distally until a hard stop. The user then would unload the cartridge, e.g., by rotating the cartridge counterclockwise to stop, sliding the cartridge axially out of the shaft, and releasing the reset slider button. This procedure generally would be performed for two otomy sites intended to be joined, and then the two sites would be brought into proximity such that the two OTOLoc devices abut through magnetic coupling of the two Flexagon devices (FIG. 88).

It should be noted that the universal delivery device may be used multiple times in a single surgical procedure such as to deploy a first OTOLoc and Flexagon for a first otomy/lumen using a first cartridge and to deploy a second OTOLoc and Flexagon for a second otomy/lumen using a second cartridge, with subsequent joining of the two otomies via the deployed Flexagon devices, for example as depicted in FIGS. 23E-23F. In this regard, embodiments may provide a mechanism for controlling a suture from the first Flexagon during operation of the second cartridge (e.g., including an option for the suture line to pull through the second cartridge such that it is manipulable after deployment of the second OTOLoc and Flexagon). Among other things, such control the first suture could replace an end-to-end anastomosis (EEA) in a rectal anastomosis with little lap assistance.

It should be noted that embodiments could be configured to provide for deploying more than two devices using a single cartridge, e.g., a cartridge containing a first otomy control device distal to a first anastomosis device distal to a second otomy control device distal to a second anastomosis device, such that, for example a single cartridge could be used to deploy a first OTOLoc and Flexagon for a first otomy/lumen and then to deploy a second OTOLoc and Flexagon for a second otomy/lumen, with subsequent joining of the two otomies via the deployed Flexagon devices, for example as depicted in FIGS. 23E-23F. Such an embodiment could allow for differently configured otomy control devices (e.g., different shapes, sizes, materials, coatings, etc.) and/or differently configured anastomosis or other devices (e.g., different shapes, sizes, materials, coatings, magnetic configurations, control features, etc.).

It should be noted that certain embodiments could allow the cartridge to also form the initial otomy, e.g., by configuring the tip of the cartridge for piercing, cutting, or otherwise creating the initial otomy. The tip could include active elements (e.g., mechanical cutting elements, electromechanical cutting elements, heating elements, etc.), and an appropriate interface could be provided from the universal delivery device to the cartridge to operate and/or power such active elements. The cartridge could include other elements, e.g., a camera or other optical element to assist the surgeon with placement.

It should be noted that in any of the embodiments described with reference to an OTOLoc™ device, other types of otomy control devices could be used instead. Similarly, it should be noted that in any of the embodiments described with reference to a Flexagon™ device, other types of magnetic compression anastomosis devices or other devices could be used instead.

It also should be noted that various embodiments described herein can be configured for use or delivery via open surgical, laparoscopic, endoscopic or surgical robot-based techniques. For example, notwithstanding the handpiece being manually operatable, embodiments of the handpiece can be additionally or alternatively configured to allow for robotic manipulation, e.g., configured to allow a surgical robot to grasp/hold the handpiece and operate the one or more actuators (e.g., the deployment trigger and the suture release trigger) or including additional robot-controllable actuators corresponding at least functionally to the manual-controllable actuators.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Various embodiments of the present invention may be characterized by the potential claims listed in the paragraphs following this paragraph (and before the actual claims provided at the end of the application). These potential claims form a part of the written description of the application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public. Nor are these potential claims intended to limit various pursued claims.

Without limitation, potential subject matter that may be claimed (prefaced with the letter “P” so as to avoid confusion with the actual claims presented below) includes:

P1. A universal delivery system comprising a handpiece with shaft and one or more cartridges as shown and described.

P2. A cartridge configured for use with a handpiece as shown and described.

P3. A method of treatment using a handpiece and one or more cartridges as shown and described.

P4. A handpiece having a shaft that is configured to attach a plurality of different anastomosis procedure cartridges and through which the cartridges are operated via the handpiece.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

1. A system for delivery of one or more implants used in the protection of otomies and the creation of magnetic compression anastomoses in a target anatomy, comprising a reloadable handpiece and one or more cartridges containing the one or more implants.

2. The system of claim 1, wherein:
each cartridge contains at least one implant selected from the group consisting of an otomy control device and a magnetic compression anastomosis device; and
the reloadable handpiece has an interface (e.g., a shaft) through which each of the one or more cartridges can be attached to and operated by the handpiece, the handpiece including a deployment mechanism configured to control deployment of the at least one implant from a distal end of an attached cartridge and at least one actuator allowing a user to operate the deployment mechanism.

3. The system of claim 2, wherein at least one cartridge includes only one implant selected from the group consisting of an otomy control device and a magnetic compression anastomosis device.

4. (canceled)

5. The system of claim 2, wherein at least one cartridge includes a distal implant and a proximal implant, wherein the deployment mechanism is configured to deploy the distal implant followed by the proximal implant.

6. The system of claim 5, wherein the deployment mechanism is configured to perform a first deployment operation that deploys the distal implant and to perform a second deployment operation that deploys the proximal implant, wherein the second deployment operation requires a separate actuation from the first deployment operation.

7. The system of claim 5, wherein the distal implant is an otomy control device and the proximal implant is a magnetic compression anastomosis device.

8. The system of claim 7, wherein the cartridge contains the otomy control device distal to the magnetic compression anastomosis device without otomy alignment, wherein the cartridge deploys the otomy control device and then requires access of the otomy control device prior to deployment of the magnetic compression anastomosis device through the otomy control device.

9. The system of claim 7, wherein the cartridge contains the otomy control device distal to the magnetic compression anastomosis device with thru deployment, wherein the cartridge deploys a distal flange of the otomy control device and then deploys the magnetic compression anastomosis device through the otomy control device and then deploys a proximal flange of the otomy control device.

10. The system of claim 7, wherein the cartridge contains the otomy control device distal to the magnetic compression anastomosis device with alignment, wherein the cartridge deploys the otomy control device and maintains a connection with the otomy control device for deployment of the magnetic compression anastomosis device through the otomy control device and subsequent release of the otomy control device.

11. The system of claim 5, wherein the distal implant is a magnetic compression anastomosis device and the proximal implant is an otomy control device.

12. The system of claim 2, wherein at least one cartridge includes a plurality of otomy control devices and/or a plurality of magnetic compression anastomosis devices.

13. The system of claim 12, wherein the cartridge includes in order a distal otomy control device, a distal magnetic compression anastomosis device, a proximal otomy control device, and a proximal magnetic compression anastomosis device.

14. The system of claim 2, wherein the at least one actuator includes an implant delivery actuator and a suture release actuator.

15. The system of claim 2, wherein the deployment mechanism comprises at least one of:
a pusher configured to push the at least one implant from the distal end of the deployment channel;
a ratcheting feature to control advancement of the at least one implant;
a linkage drive mechanism for controlling actuation and release operations;
a cable drive mechanism for controlling actuation and release operations; or
one or more motors for controlling actuation and release operations.

16-19. (canceled)

20. The system of claim 2, wherein the magnetic compression anastomosis device is a self-assembling magnetic compression anastomosis device having a substantially linear configuration within the cartridge and an annular deployed configuration, and wherein the otomy control device is an OTOLoc™ otomy control device.

21. (canceled)

22. An anastomosis procedure cartridge containing at least one implant selected from the group consisting of an otomy control device and a magnetic compression anastomosis device, wherein the cartridge is configured to attach to an interface of a reloadable handpiece by which the cartridge can be operated to deliver and release the at least one implant from a distal end of the cartridge.

23-24. (canceled)

25. The cartridge of claim 22, wherein the at least one implant includes a distal implant and a proximal implant.

26-34. (canceled)

35. A reloadable handpiece comprising an interface configured to attach to and operate an anastomosis procedure cartridge containing at least one implant selected from the group consisting of an otomy control device and a magnetic compression anastomosis device, the handpiece including a deployment mechanism configured to control deployment of the at least one implant from a distal end of an attached cartridge, the deployment mechanism including at least one actuator for a user to operate the deployment mechanism.

36. (canceled)

37. The handpiece of claim 35, wherein the cartridge includes a distal implant and a proximal implant, and wherein the deployment mechanism is configured to perform a first deployment operation that deploys the distal implant and to perform a second deployment operation that deploys the proximal implant, wherein the second deployment operation requires a separate actuation from the first deployment operation.

38-53. (canceled)

54. The handpiece of claim 35, wherein the handpiece is configured for robotic operation in addition to or in lieu of manual operation.
